(19) United States
(12) Patent Application Publication
(10) Pub. No.: US 2026/0062421 A1
(43) Pub. Date: Mar. 5, 2026

(54) NOVEL PROCESSES FOR PREPARING CAMPTOTHECIN DERIVATIVES

(71) Applicant: IMMUNOGEN, INC., Waltham, MA (US)

(72) Inventors: Feng Xu, Changzhou (CN); Mariya Volodimirivna Kozytska, Waltham, MA (US); Andrew Brian Hague, Chelmsford, MA (US); Mark Cameron, Brick, NJ (US); Lakshindra Chetia, Bangalore (IN); Ravikumar Katupalayam, Bangalore (IN); Ramesh Annadasu, Bangalore (IN); Sudhir Tulshiram Patil, Changzhou (CN); Jiangkun Zhang, Changzhou (CN); Bhowmick Sudipto, Changzhou (CN)

(21) Appl. No.: 18/997,738

(22) PCT Filed: Jul. 25, 2023
(86) PCT No.: PCT/US2023/028593

§ 371 Date: Jan. 22, 2025

(30) Foreign Application Priority Data
Jul. 25, 2022 (WO) PCT/CN2022/107665

(51) Int. Cl. C07D 491/22; C07K 5/062; C07K 5/083; C07K 5/103

(52) U.S. Cl. CPC C07D 491/22; C07K 5/06026; C07K 5/06052; C07K 5/0806; C07K 5/1008

(57) ABSTRACT

The invention provides novel methods for preparing camptothecin derivatives and their synthetic precursors.

### RELATED APPLICATION

This application claims priority to International Application No. PCT/CN2022/107665, filed on Jul. 25, 2022, the entire content of which is incorporated herein by reference.

### FIELD OF THE INVENTION

The present invention relates to novel methods for preparing camptothecin derivatives.

### BACKGROUND OF THE INVENTION

Cell binding agent-drug conjugates, including antibody-drug conjugates (ADC) are emerging as a powerful class of agents with efficacy across a range of abnormal cell growth or proliferative diseases (e.g., cancers). Cell binding agent-drug conjugates (such as ADCs) are commonly composed of three distinct elements: a cell-binding agent (e.g., an antibody); a linker; and a cytotoxic moiety.

Camptothecin (CPT) is a pentacyclic alkaloid isolated from the bark and stem of Camptotheca acuminata (Camptotheca, Happy tree), a tree native to China. Camptothecin inhibits topoisomerase I, which leads to cell death. Because of its cytotoxic mechanism and broad-spectrum antitumor activity, there have been substantial efforts towards developing clinical analogues of camptothecin. It has been found that conjugates of camptothecin derivatives and cell binding agents are useful as medicaments, in particular as anti-proliferative agents (anticancer agents).

There exists a need to develop new processes that are efficient and more suitable for large scale manufacturing of camptothecin derivatives.

### SUMMARY OF THE INVENTION

The processes described herein have higher reaction yield and/or product purity profile, which renders these processes more suitable for large scale manufacture. In some embodiments, the processes describe herein do not require the use of column chromatography for product purification, which makes the processes more scalable and suitable for large scale manufacturing. In some embodiments, the processes described herein incorporate the camptothecin chemical moiety at late stage of the processes, thereby reducing the workers' exposure time to highly toxic chemical materials.

In one embodiment, the present invention provides a method of preparing a compound of Formula (II):

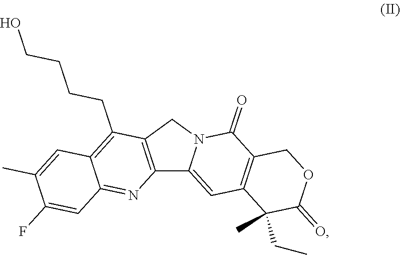 [SMILES: CC[C@@]1(C)C(=O)OCc2c1cc1n(c2=O)Cc2c-1nc1cc(F)c(C)cc1c2CCCCO]

comprising the step of reacting a compound of Formula (I):

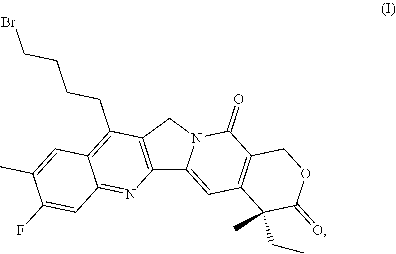 [SMILES: CC[C@@]1(C)C(=O)OCc2c1cc1n(c2=O)Cc2c-1nc1cc(F)c(C)cc1c2CCCCBr]

with water and N-methyl-2-pyrollidone (NMP) to form the compound of Formula (II).

In another embodiment, the present invention provides a method of preparing a compound of Formula (III):

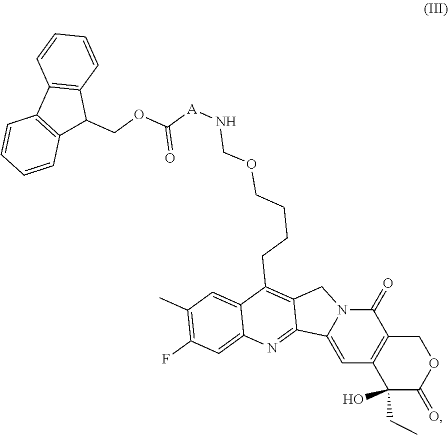

comprising reacting a compound of Formula (II):

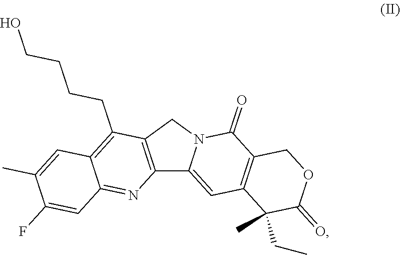 [SMILES: CC[C@@]1(C)C(=O)OCc2c1cc1n(c2=O)Cc2c-1nc1cc(F)c(C)cc1c2CCCCO]

with a compound of Formula (A):

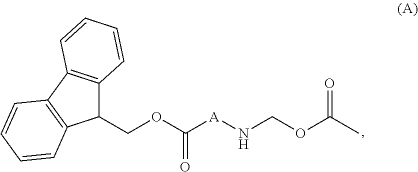

in the presence of trifluoroacetic acid (TFA) to form the compound of Formula (III), wherein A is a peptide comprising 2 to 10 amino acids.

In another embodiment, the present invention provides a method of preparing a compound of Formula (III):

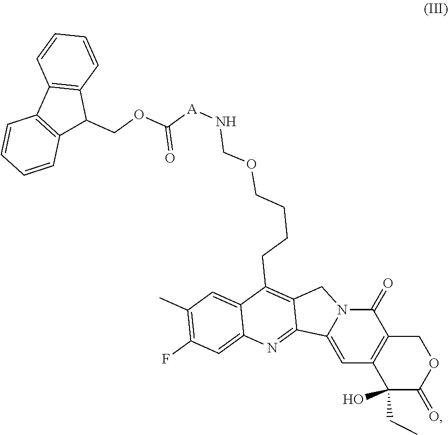

comprising reacting a compound of Formula (II):

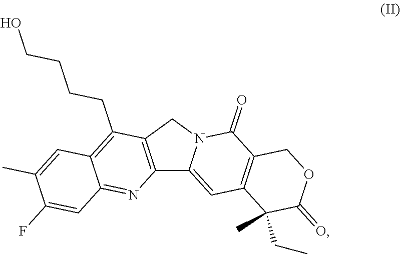 [SMILES: CC[C@@]1(C)C(=O)OCc2c1cc1n(c2=O)Cc2c-1nc1cc(F)c(C)cc1c2CCCCO]

with a compound of Formula (A):

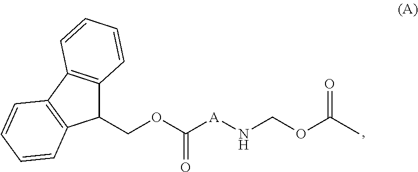

in the presence of a Lewis acid to form the compound of Formula (III), wherein A is a peptide comprising 2 to 10 amino acids.

In another embodiment, the present invention provides a method of preparing a compound of Formula (VII):

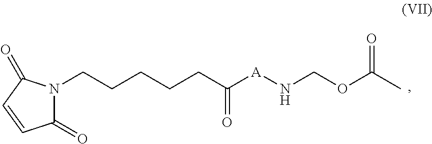

comprising the steps of:
(a) deprotecting a compound of Formula (A):

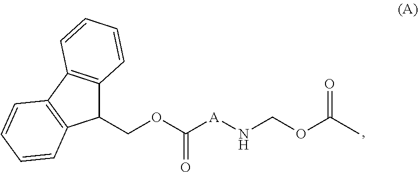

with a base to form a compound of Formula (VI):

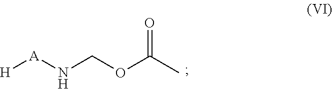

(b) reacting the compound of Formula (VI) with a compound of Formula (B):

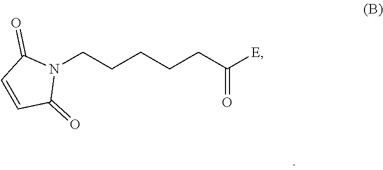

wherein A is a peptide comprising 2 to 10 amino acids; and E is —OH, —Cl or

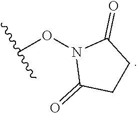 [SMILES: *ON1C(=O)CCC1=O]

In yet another embodiment, the present invention provides a method of preparing a compound of Formula (V):

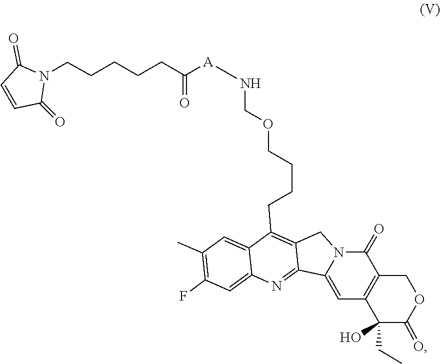

comprising reacting a compound of Formula (VII):

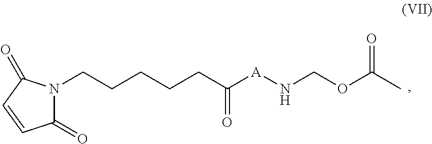

with a compound of Formula (II):

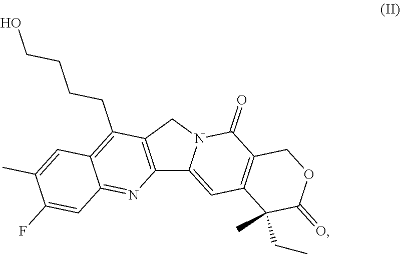 [SMILES: CC[C@@]1(C)C(=O)OCc2c1cc1n(c2=O)Cc2c-1nc1cc(F)c(C)cc1c2CCCCO]

to form the compound of Formula (V).

In yet another embodiment, the present invention provides a method of preparing a compound of Formula (II):

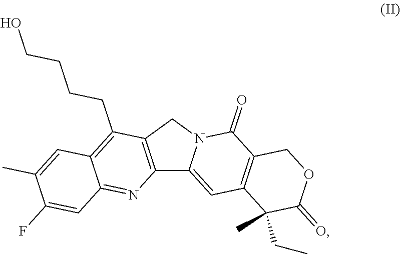 [SMILES: CC[C@@]1(C)C(=O)OCc2c1cc1n(c2=O)Cc2c-1nc1cc(F)c(C)cc1c2CCCCO]

comprising reacting a compound of Formula (VIII):

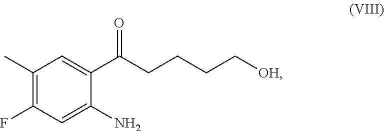 [SMILES: Cc1cc(C(=O)CCCCO)c(N)cc1F]

with a compound of Formula (C):

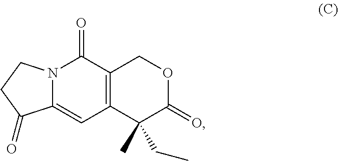 [SMILES: CC[C@@]1(C)C(=O)OCc2c1cc1n(c2=O)CCC1=O]

to form the compound of Formula (II).

### DETAILED DESCRIPTION OF THE INVENTION

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents which may be included within the scope of the present invention as defined by the claims. One skilled in the art will recognize many methods and materials similar or equivalent to those described herein, which could be used in the practice of the present invention.

It should be understood that any of the embodiments described herein can be combined with one or more other embodiments of the invention, unless explicitly disclaimed or improper. Combination of embodiments are not limited to those specific combinations claimed via the multiple dependent claims.

### Definitions

Unless otherwise defined herein, scientific and technical terms used in this application shall have the meanings that are commonly understood by those of ordinary skill in the art to which this invention belongs. Generally, nomenclature used in connection with the compounds, composition and methods described herein, are those well-known and commonly used in the art.

Chemistry terms used herein are used according to conventional usage in the art, as exemplified by “The McGraw-Hill Dictionary of Chemical Terms”, Parker S., Ed., McGraw-Hill, San Francisco, C.A. (1985).

The term “herein” means the entire application.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

Throughout the specification, where compositions are described as having, including, or comprising (or variations thereof), specific components, it is contemplated that compositions also may consist essentially of, or consist of, the recited components. Similarly, where methods or processes are described as having, including, or comprising specific process steps, the processes also may consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

The term “including” is used to mean “including but not limited to.” “Including” and “including but not limited to” are used interchangeably.

As used herein, “about” or “approximately” means within an acceptable error range for the particular value as determined by one of ordinary skill in the art, which will depend in part on how the value is measured or determined, i.e., the limitations of the measurement system.

The use of the terms “a” and “an” and “the” and similar referents in the context of describing the elements (especially in the context of the following claims) are to be construed to cover both the singular and the plural, unless otherwise indicated herein or clearly contradicted by context.

The term “or” as used herein should be understood to mean “and/or.” unless the context clearly indicates otherwise.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

The term “compound” is intended to include compounds for which a structure or formula or any derivative thereof has been disclosed in the present invention or a structure or formula or any derivative thereof that has been incorporated by reference. The term also includes, stereoisomers, geometric isomers, tautomers, solvates, and salts (e.g., pharmaceutically acceptable salts) of a compound of all the formulae disclosed in the present invention. The term also includes any solvates, hydrates, and polymorphs of any of the foregoing. The specific recitation of “stereoisomers,” “geometric isomers,” “tautomers,” “solvates,” “salt” “hydrate,” or “polymorph” in certain aspects of the invention described in this application shall not be interpreted as an intended omission of these forms in other aspects of the invention where the term “compound” is used without recitation of these other forms.

The term “chiral” refers to molecules that have the property of non-superimposability of the mirror image partner, while the term “achiral” refers to molecules that are superimposable on their mirror image partner.

The term “stereoisomer” refers to compounds that have identical chemical constitution and connectivity, but different orientations of their atoms in space that cannot be interconverted by rotation about single bonds.

The term “diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers can separate under high resolution analytical procedures such as crystallization, electrophoresis and chromatography.

The term “enantiomers” refer to two stereoisomers of a compound that are non-superimposable mirror images of one another. Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds,” John Wiley & Sons, Inc., New York, 1994. The compounds of the invention can contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

The term “tautomer” or “tautomeric form” refers to structural isomers of different energies that are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

The term “salt” as used herein, refers to an organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts. A salt can involve the inclusion of another molecule such as an acetate ion, a succinate ion or other counter ion. The counter ion can be any organic or inorganic moiety that stabilizes the charge on the parent compound. Furthermore, a salt can have more than one charged atom in its structure. Instances where multiple charged atoms are part of the salt can have multiple counter ions. Hence, a salt can have one or more charged atoms and/or one or more counter ion.

If the compound of the invention is a base, the desired salt can be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the compound of the invention is an acid, the desired salt can be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

In certain embodiments, the salt is a pharmaceutically acceptable salt. The phrase “pharmaceutically acceptable” indicates that the substance or composition must be compatible chemically and/or toxicologically, with the other ingredients comprising a formulation, and/or the mammal being treated therewith.

The term “solvate” means a compound that further includes a stoichiometric or non-stoichiometric amount of solvent such as water, isopropanol, acetone, ethanol, methanol, DMSO, ethyl acetate, acetic acid, and ethanolamine dichloromethane, 2-propanol, or the like, bound by non-covalent intermolecular forces. Solvates or hydrates of the compounds are readily prepared by addition of at least one molar equivalent of a hydroxylic solvent such as methanol, ethanol, 1-propanol, 2-propanol or water to the compound to result in solvation or hydration of the imine moiety.

The term “amino acid” refers to naturally occurring amino acids or non-naturally occurring amino acid. In some embodiments, the amino acid is represented by NH₂—C(R^{aa′}R^{aa})—C(═O)OH, wherein R^{aa} and R^{aa′} are each independently H, an optionally substituted linear, branched or cyclic alkyl, alkenyl or alkynyl having 1 to 10 carbon atoms, aryl, heteroaryl or heterocyclyl or R^{aa} and the N-terminal nitrogen atom can together form a heterocyclic ring (e.g., as in proline). The term “amino acid residue” refers to the corresponding residue when one hydrogen atom is removed from the amine and/or carboxy end of the amino acid, such as —NH—C(R^{aa′}R^{aa})—C(═O)—.

The term “peptide” refers to short chains of amino acid monomers linked by peptide (amide) bonds. In some embodiments, the peptides contain 2 to 20 amino acid residues. In other embodiments, the peptides contain 2 to 10 or 2 to 8 amino acid residues. In yet other embodiments, the peptides contain 2 to 5 amino acid residues. As used herein, when a peptide is a portion of a cytotoxic agent or a linker described herein represented by a specific sequence of amino acids, the peptide can be connected to the rest of the cytotoxic agent or the linker in both directions.

The term “immunoconjugate.” “conjugate.” or “ADC” as used herein refers to a compound or a derivative thereof that is linked to a cell binding agent (e.g., an antibody or antigen-binding fragment thereof).

The term “cell-binding agent” in the immunoconjugates of the present invention can be of any kind presently known, or that become known, including peptides and non-peptides that binds to a cell or cell component (e.g., receptor, protein, DNA, RNA, etc.). Generally, these can be antibodies (such as polyclonal antibodies and monoclonal antibodies, especially monoclonal antibodies) or fragments thereof, lymphokines, hormones, growth factors, vitamins (such as folate etc., which can bind to a cell surface receptor thereof, e.g., a folate receptor), nutrient-transport molecules (such as transferrin), probodies, nanobodies, or any other cell-binding molecule or substance.

The term “cation” refers to an ion with positive charge. The cation can be monovalent (e.g., Na⁺, K⁺, etc.), bi-valent (e.g., Ca²⁺, Mg²⁺, etc.) or multi-valent (e.g., Al³⁺ etc.). Preferably, the cation is monovalent.

The term “acid” refers to any substance that in water solution tastes sour, changes the color of certain indicators (e.g., reddens blue litmus paper), reacts with some metals (e.g., iron) to liberate hydrogen, reacts with bases to form salts, and promotes certain chemical reactions (acid catalysis). Examples of acids include the inorganic substances known as the mineral acids—sulfuric, nitric, hydrochloric, and phosphoric acids—and the organic compounds belonging to the carboxylic acid, sulfonic acid, and phenol groups. Such substances contain one or more hydrogen atoms that, in solution, are released as positively charged hydrogen ions. Examples of acid includes trifluoroacetic acid (TFA), pyridinium p-toluenesulfonate (PPTS), p-toluenesulfonic acid, methanesulfonic acid, camphorsulfonic acid, sulfuric acid, hydrochloric acid (HCl), and trichloroacetic acid.

The term “base” refers to a substance that can accept hydrogen ions (protons) or donate a pair of valence electrons. Examples of the suitable bases include piperidine, morpholine, N-methylmorpholine, 4-methylpiperidine, piperazine, pyrrolidine, 1,8-diazabicyclo[5.4.0]undec-7-ene (DBU), diethylamine (DEA), a trialkylamine (e.g., diisopropylethylamine (DIPEA), triethylamine (TEA), and 1,8-Diazabicycloundec-7-ene), a metal alkoxide (e.g., sodium tert-butoxide and potassium tert-butoxide), an alkyl metal (e.g., tert-butyllithium, methyl lithium, n-butyl lithium, tert-butyl lithium, lithium di-isopropylamide, pentyl sodium, and 2-phenyl isopropyl-potassium), an aryl metal (e.g., phenyl lithium), a metal hydride (e.g., sodium hydride), a metal amide (e.g., sodium amide, potassium amide, lithium diisopropylamide and lithium tetramethylpiperidide), and a silicon-based amide (e.g., sodium bis(trimethylsilyl) amide and potassium bis(trimethylsilyl) amide).

The term “Lewis acid” refers to an acid substance which can employ an electron lone pair from another molecule in completing the stable group of one of its own atoms. Exemplary Lewis acids for use in the disclosed methods include boron trifluoride etherate (BF₃·OEt₂), zinc triflate, zinc chloride, magnesium bromide, magnesium triflate, copper triflate, copper (II) bromide, copper (II) chloride, magnesium chloride, and aluminum chloride (AlCl₃).

The term “catalyst” refers to a substance that increases the rate of a chemical reaction without itself undergoing any permanent chemical change.

The term “organic solvent” refers to carbon-based substances capable of dissolving or dispersing one or more other substances. Organic solvents can be carcinogens, reproductive hazards, and neurotoxins. Carcinogenic organic solvents include benzene, carbon tetrachloride, and trichloroethylene. Organic solvents recognized as reproductive hazards include 2-ethoxyethanol, 2-methoxyethanol, and methyl chloride. Organic solvents recognized as neurotoxins include n-hexane, tetrachloroethylene, and toluene. Many classes of chemicals are used as organic solvents, including aliphatic hydrocarbons, aromatic hydrocarbons, amines, esters, ethers, ketones, and nitrated or chlorinated hydrocarbons. Exemplary organic solvents include dichloromethane (CH₂Cl₂ or DCM), dichloroethane (DCE), acetonitrile (ACN or MeCN), methanol (MeOH), tetrahydrofuran (THF), toluene, N-methylmorpholine (NMM), imethylformamide (DMF), Dimethyl sulfoxide (DMSO), dimethylacetamide (DMA or DMAc), or any combination thereof.

### Methods of the Present Invention

The present invention provides novel methods for preparing camptothecin derivatives that can be linked to cell binding agents (e.g., antibodies) to form immunoconjugates which are useful as medicaments, in particular as anti-proliferative agents (anticancer agents).

In a first embodiment, the present invention provides a method of preparing a compound of Formula (II):

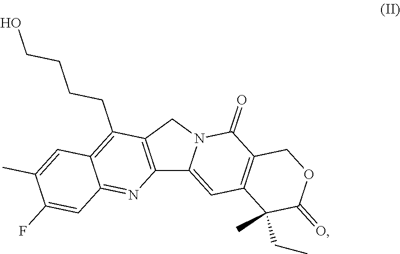 [SMILES: CC[C@@]1(C)C(=O)OCc2c1cc1n(c2=O)Cc2c-1nc1cc(F)c(C)cc1c2CCCCO]

comprising the step of reacting a compound of Formula (I):

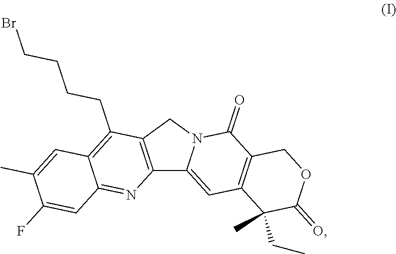 [SMILES: CC[C@@]1(C)C(=O)OCc2c1cc1n(c2=O)Cc2c-1nc1cc(F)c(C)cc1c2CCCCBr]

with water and N-methyl-2-pyrollidone (NMP) to form the compound of Formula (II).

The reaction between the compound of Formula (I) and NMP can be carried out at a suitable temperature. In some embodiments, the reaction is carried out at a temperature between 20° C. and 150° C. In some embodiments, the reaction is carried out at a temperature between 30° C. and 150° C., between 70° C. and 120° C., or between 80° C. and 120° C. In more specific embodiments, the reaction is carried out at 100° C.

Suitable amount of NMP can be used in the reaction between the compound of Formula (I) and NMP. In some embodiments, excess amount of NMP relative to the compound of Formula (I) is used. In some embodiments, between 1 to 10, between 1 to 5, between 1 to 3, between 1 to 2, or between 1 to 1.5 molar equivalents of NMP relative to the compound of Formula (I) is used.

In a second embodiment, the compound of Formula (I) in the first embodiment is prepared by reacting a compound of Formula (C):

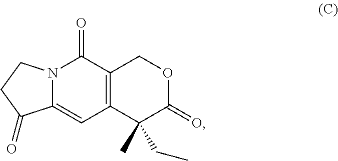 [SMILES: CC[C@@]1(C)C(=O)OCc2c1cc1n(c2=O)CCC1=O]

with a compound of Formula (D):

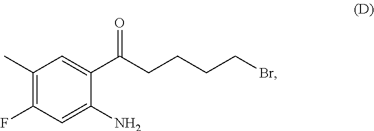 [SMILES: Cc1cc(C(=O)CCCCBr)c(N)cc1F]

to form the compound of Formula (I).

In some embodiments, the reaction between the compound of Formula (C) and the compound of Formula (D) is carried out in the presence of pyridinium p-toluenesulfonate (PPTS). In some embodiments, the reaction between the compound of Formula (C) and the compound of Formula (D) is carried out in an organic solvent, such as toluene.

In a third embodiment, the compound of Formula (D) in the second embodiment is prepared by reacting a compound of Formula (E) and a compound of Formula (F):

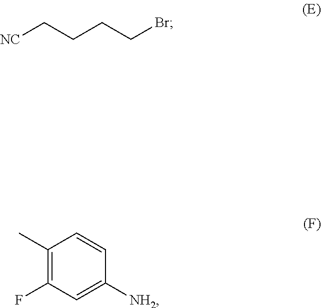

to form the compound of Formula (D).

In some embodiments, the reaction between the compound of Formula (E) and the compound of Formula (F) is carried out in the presence of boron trichloride (BCl₃) and aluminum trichloride (AlCl₃), boron trichloride (BCl₃) and aluminum tribromide (AlBr₃), boron tribromide (BBr₃) and aluminum tribromide (AlBr₃), or boron trichloride (BCl₃) and zinc chloride (ZnCl₂). In some embodiments, the reaction between the compound of Formula (E) and the compound of Formula (F) is carried out in an organic solvent, such as CH₂Cl₂ or chlorobenzene (PhCl).

In a fourth embodiment, the present invention provides a method of preparing a compound of Formula (III):

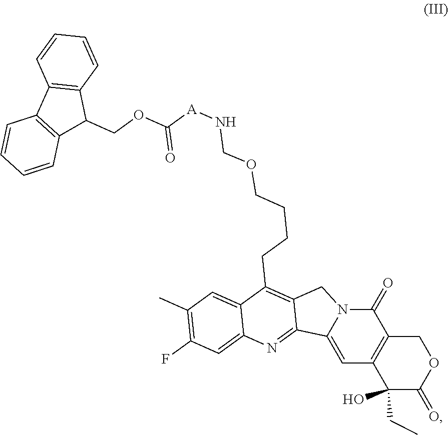

comprising reacting a compound of Formula (II):

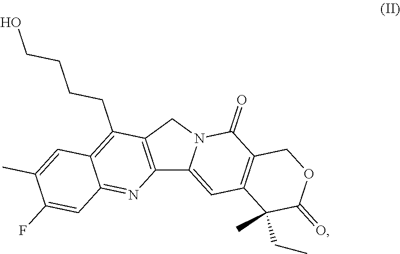 [SMILES: CC[C@@]1(C)C(=O)OCc2c1cc1n(c2=O)Cc2c-1nc1cc(F)c(C)cc1c2CCCCO]

with a compound of Formula (A):

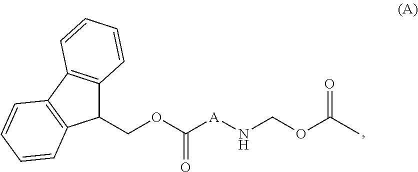

in the presence of trifluoroacetic acid (TFA) to form the compound of Formula (III), wherein A is a peptide comprising 2 to 10 amino acids.

Any suitable solvents can be used for the reaction in the fourth embodiment. In some embodiments, the solvent is dimethylformamide (DMF).

In a fifth embodiment, the present invention provides method of preparing a compound of Formula (III):

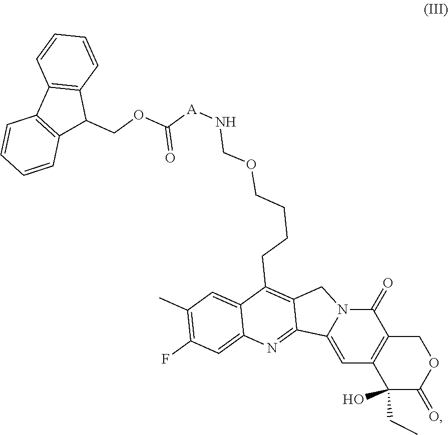

comprising reacting a compound of Formula (II):

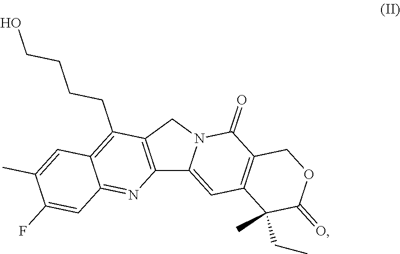 [SMILES: CC[C@@]1(C)C(=O)OCc2c1cc1n(c2=O)Cc2c-1nc1cc(F)c(C)cc1c2CCCCO]

with a compound of Formula (A):

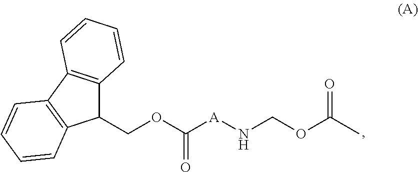

in the presence of a Lewis acid to form the compound of Formula (III), wherein A is a peptide comprising 2 to 10 amino acids.

The reaction between the compound of Formula (II) and the compound of Formula (A) in the fifth embodiment can be carried out in the presence of any suitable Lewis acids. In some embodiments, the Lewis acid is boron trifluoride etherate (BF₃·OEt₂), boron trichloride (BCl₃), or aluminum trichloride (AlCl₃). In some embodiments, the Lewis acid is boron trifluoride etherate (BF₃·OEt₂).

Any suitable solvents can be used for the reaction in the fifth embodiment. In some embodiments, the solvent is dimethyl sulfoxide (DMSO).

In some embodiments, the peptide represented by A in the fourth or fifth embodiment is a peptide comprising 2 to 4 amino acids. In some embodiments, A is -Ala-Ala-*, -Ala-Val-*, -Val-Ala-*, -Val-Cit-*, -Cit-Val-*, -Gln-Leu-*, -Leu-Gln-*, -Ala-Ala-Ala-*, -Ala-Ala-Ala-Ala-*, -Gly-Ala-Gly-Gly-*, -Gly- Gly-Ala-Gly-*, -Gly-Val-Gly-Gly-*, -Gly-Gly-Val-Gly-*, -Gly-Phe-Gly-Gly-*, or -Gly-Gly-Phe-Gly-*, wherein * is the point of attachment to the carbonyl (—C(═O)—) group in Formula (A). In some embodiments, A is -Val-Cit-*, -Cit-Val-*, -Ala-Ala-Ala-* or -Gly-Phe-Gly-Gly-*, -Gly-Gly-Phe-Gly-*.

In a sixth embodiment, the compound of Formula (III) in the fourth or fifth embodiment is represented by Formula (IIIa):

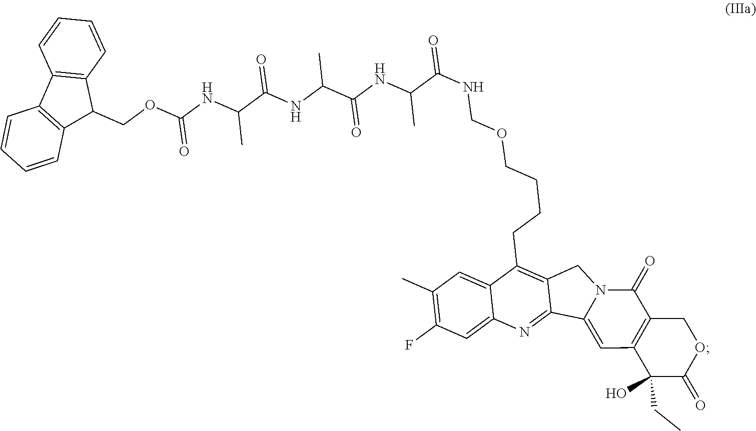 [SMILES: CC[C@@]1(O)C(=O)OCc2c1cc1n(c2=O)Cc2c-1nc1cc(F)c(C)cc1c2CCCCOCNC(=O)C(C)NC(=O)C(C)NC(=O)C(C)NC(=O)OCC1c2ccccc2-c2ccccc21]

and
the compound of Formula (A) in the fourth or fifth embodiment is represented by Formula (Aa):

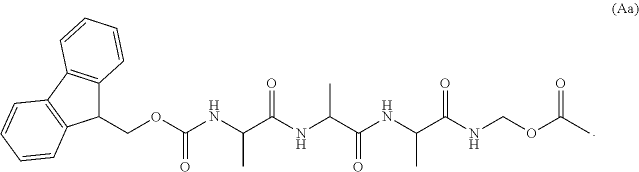 [SMILES: CC(=O)OCNC(=O)C(C)NC(=O)C(C)NC(=O)C(C)NC(=O)OCC1c2ccccc2-c2ccccc21]

In a 1^{st} specific embodiment, the compound of Formula (IIIa) in the sixth embodiment is represented by Formula (IIIa-1):

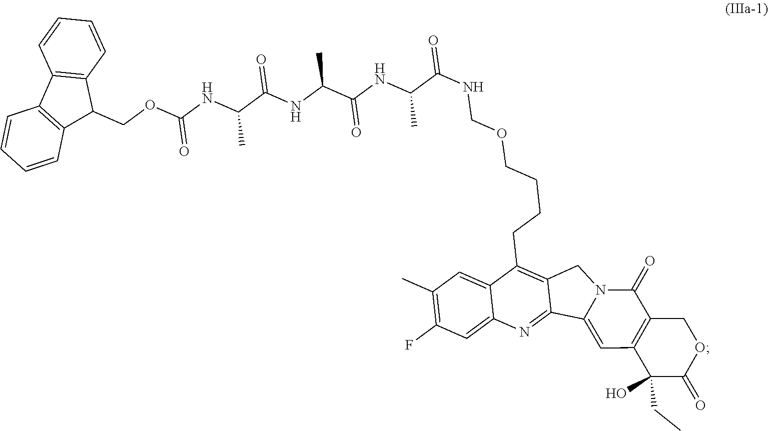 [SMILES: CC[C@@]1(O)C(=O)OCc2c1cc1n(c2=O)Cc2c-1nc1cc(F)c(C)cc1c2CCCCOCNC(=O)[C@H](C)NC(=O)[C@H](C)NC(=O)[C@H](C)NC(=O)OCC1c2ccccc2-c2ccccc21]

and
the compound of Formula (Aa) in the sixth embodiment is represented by Formula (Aa-1):

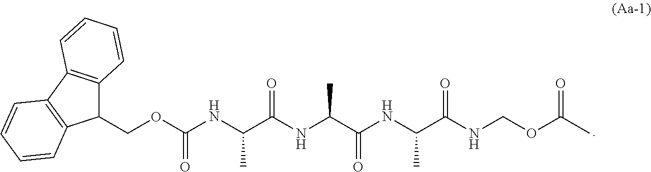 [SMILES: CC(=O)OCNC(=O)[C@H](C)NC(=O)[C@H](C)NC(=O)[C@H](C)NC(=O)OCC1c2ccccc2-c2ccccc21]

In a 2^{nd} specific embodiment, the compound of Formula (IIIa) in the sixth embodiment is represented by Formula (IIIa-2):

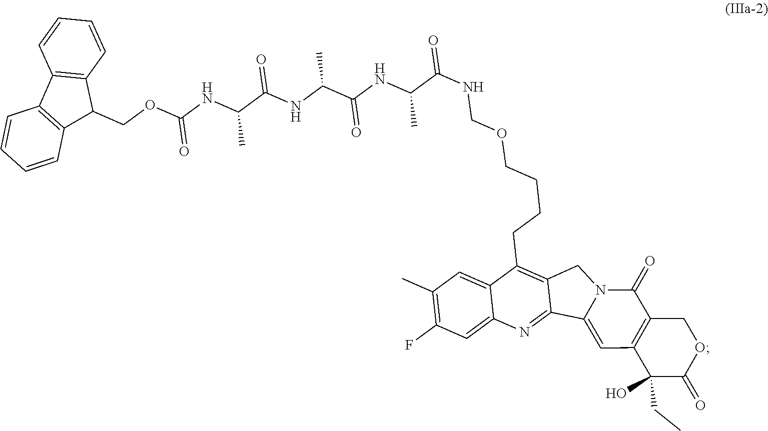 [SMILES: CC[C@@]1(O)C(=O)OCc2c1cc1n(c2=O)Cc2c-1nc1cc(F)c(C)cc1c2CCCCOCNC(=O)[C@H](C)NC(=O)[C@@H](C)NC(=O)[C@H](C)NC(=O)OCC1c2ccccc2-c2ccccc21]

and
the compound of Formula (Aa) in the sixth embodiment is represented by Formula (Aa-2):

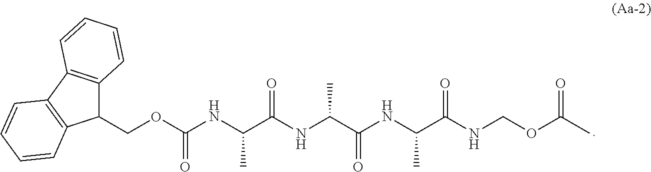 [SMILES: CC(=O)OCNC(=O)[C@H](C)NC(=O)[C@@H](C)NC(=O)[C@H](C)NC(=O)OCC1c2ccccc2-c2ccccc21]

In a seventh embodiment, the compound of Formula (III) in the fourth or fifth embodiment is represented by Formula (IIIb):

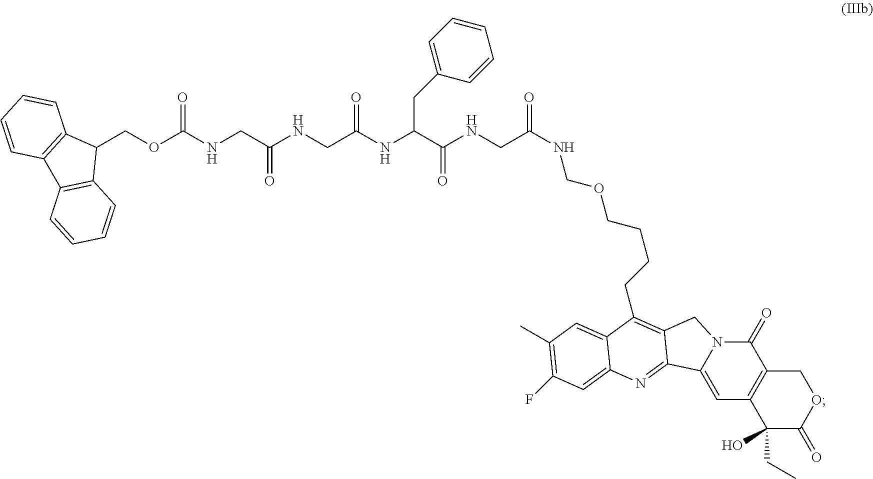 [SMILES: CC[C@@]1(O)C(=O)OCc2c1cc1n(c2=O)Cc2c-1nc1cc(F)c(C)cc1c2CCCCOCNC(=O)CNC(=O)C(Cc1ccccc1)NC(=O)CNC(=O)CNC(=O)OCC1c2ccccc2-c2ccccc21]

and
the compound of Formula (A) in the fourth or fifth embodiment is represented by Formula (Ab):

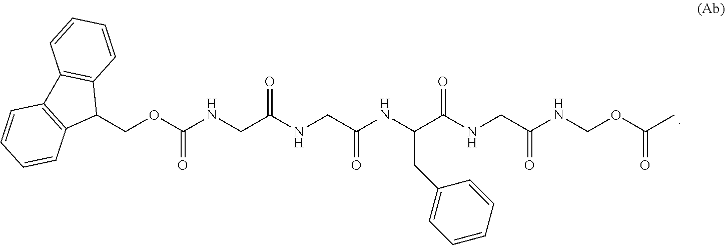 [SMILES: CC(=O)OCNC(=O)CNC(=O)C(Cc1ccccc1)NC(=O)CNC(=O)CNC(=O)OCC1c2ccccc2-c2ccccc21]

In a 3^{rd} specific embodiment, the compound of Formula (IIIb) in the seventh embodiment is represented by Formula (IIIb-1):

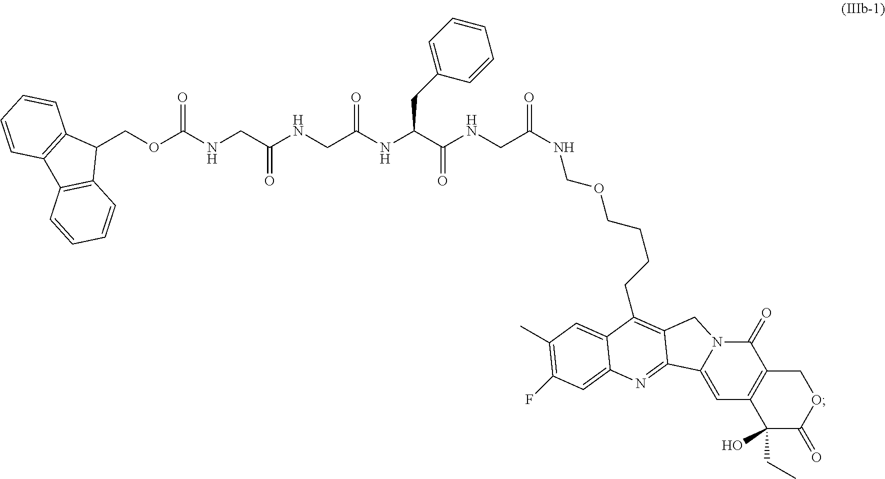 [SMILES: CC[C@@]1(O)C(=O)OCc2c1cc1n(c2=O)Cc2c-1nc1cc(F)c(C)cc1c2CCCCOCNC(=O)CNC(=O)[C@H](Cc1ccccc1)NC(=O)CNC(=O)CNC(=O)OCC1c2ccccc2-c2ccccc21]

and
the compound of Formula (Ab) in the seventh embodiment is represented by Formula (Ab-1):

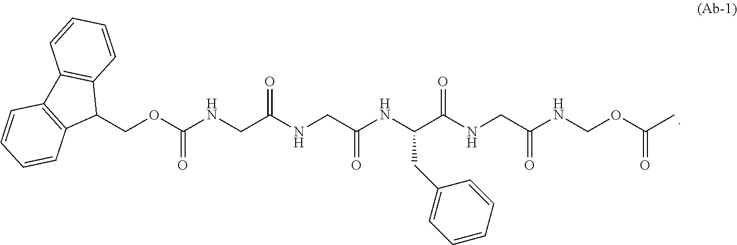 [SMILES: CC(=O)OCNC(=O)CNC(=O)[C@H](Cc1ccccc1)NC(=O)CNC(=O)CNC(=O)OCC1c2ccccc2-c2ccccc21]

In an eighth embodiment, the compound of Formula (III) in the fourth or fifth embodiment is represented by Formula (IIIc):

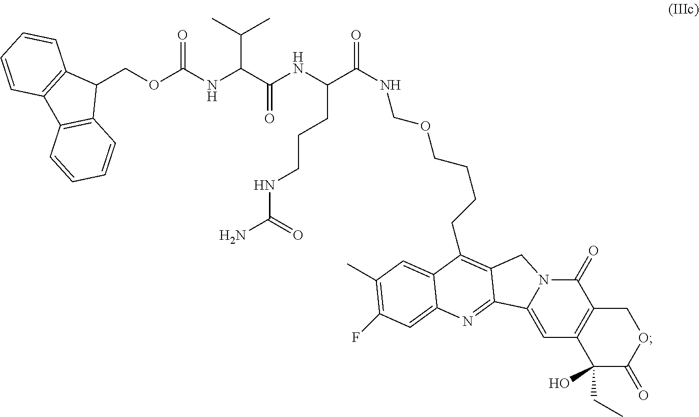 [SMILES: CC[C@@]1(O)C(=O)OCc2c1cc1n(c2=O)Cc2c-1nc1cc(F)c(C)cc1c2CCCCOCNC(=O)C(CCCNC(N)=O)NC(=O)C(NC(=O)OCC1c2ccccc2-c2ccccc21)C(C)C]

and
the compound of Formula (A) in the fourth or fifth embodiment is represented by Formula (Ac):

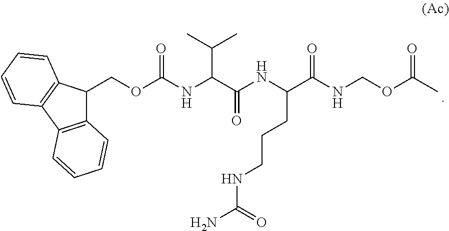 [SMILES: CC(=O)OCNC(=O)C(CCCNC(N)=O)NC(=O)C(NC(=O)OCC1c2ccccc2-c2ccccc21)C(C)C]

In a 4^{th} specific embodiment, the compound of Formula (IIIc) in the eighth embodiment is represented by Formula (IIIc-1):

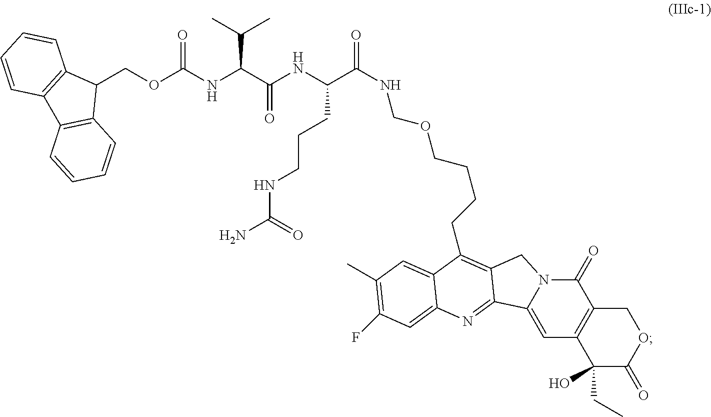 [SMILES: CC[C@@]1(O)C(=O)OCc2c1cc1n(c2=O)Cc2c-1nc1cc(F)c(C)cc1c2CCCCOCNC(=O)[C@H](CCCNC(N)=O)NC(=O)[C@@H](NC(=O)OCC1c2ccccc2-c2ccccc21)C(C)C]

and
the compound of Formula (Ac) in the eighth embodiment is represented by Formula (Ac-1):

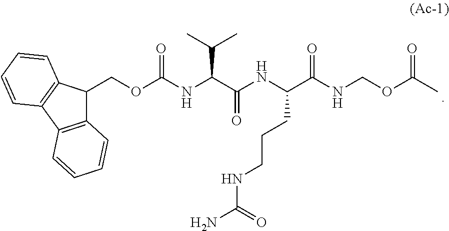 [SMILES: CC(=O)OCNC(=O)[C@H](CCCNC(N)=O)NC(=O)[C@@H](NC(=O)OCC1c2ccccc2-c2ccccc21)C(C)C]

In a ninth embodiment, the compound of Formula (III) in the fourth, fifth, sixth, 1^{st} specific, 2^{nd} specific, seventh, 3^{rd} specific, eighth, or 4^{th} specific embodiment is further reacted with a base to form the compound of Formula (IV):

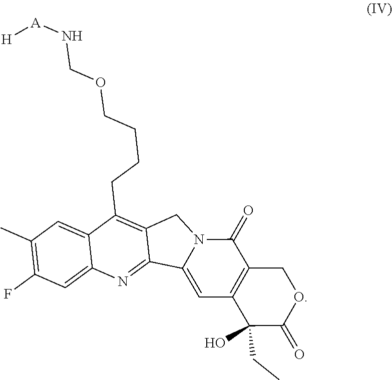

In a tenth embodiment, the compound of Formula (III) in the ninth embodiment is represented by Formula (IIIa), (IIIa-1), (IIIa-2), (IIIb), (IIIb-1), (IIIc) or (IIIc-1) and the compound of Formula (IV) in the ninth embodiment is represented by Formula (IVa), (IVa-1), (IVa-2), (IVb), (IVb-1), (IVc) or (IVc-1) respectively:

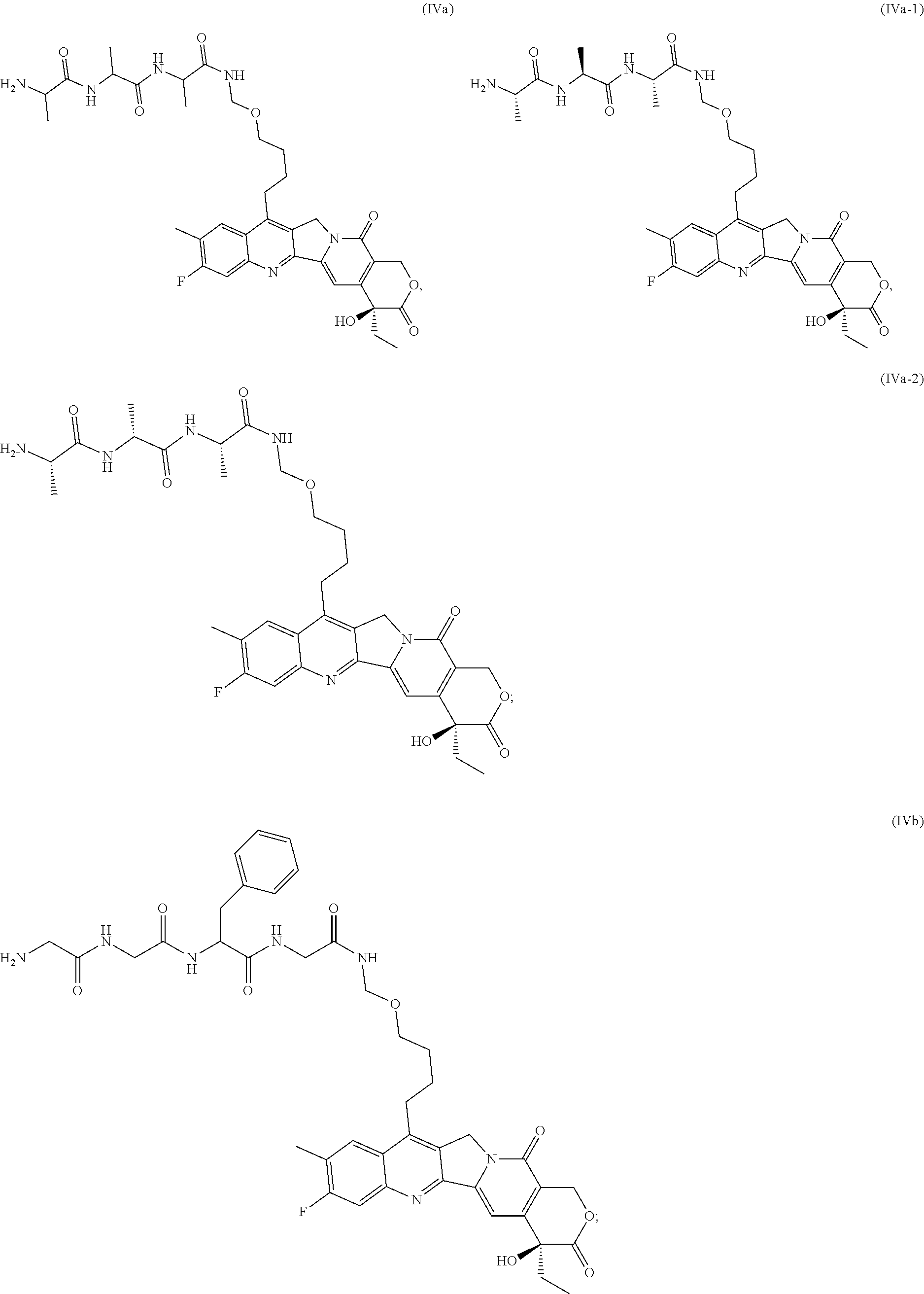 [SMILES: CC[C@@]1(O)C(=O)OCc2c1cc1n(c2=O)Cc2c-1nc1cc(F)c(C)cc1c2CCCCOCNC(=O)C(C)NC(=O)C(C)NC(=O)C(C)N ; CC[C@@]1(O)C(=O)OCc2c1cc1n(c2=O)Cc2c-1nc1cc(F)c(C)cc1c2CCCCOCNC(=O)[C@H](C)NC(=O)[C@H](C)NC(=O)[C@H](C)N ; CC[C@@]1(O)C(=O)OCc2c1cc1n(c2=O)Cc2c-1nc1cc(F)c(C)cc1c2CCCCOCNC(=O)[C@H](C)NC(=O)[C@@H](C)NC(=O)[C@H](C)N ; CC[C@@]1(O)C(=O)OCc2c1cc1n(c2=O)Cc2c-1nc1cc(F)c(C)cc1c2CCCCOCNC(=O)CNC(=O)C(Cc1ccccc1)NC(=O)CNC(=O)CN]

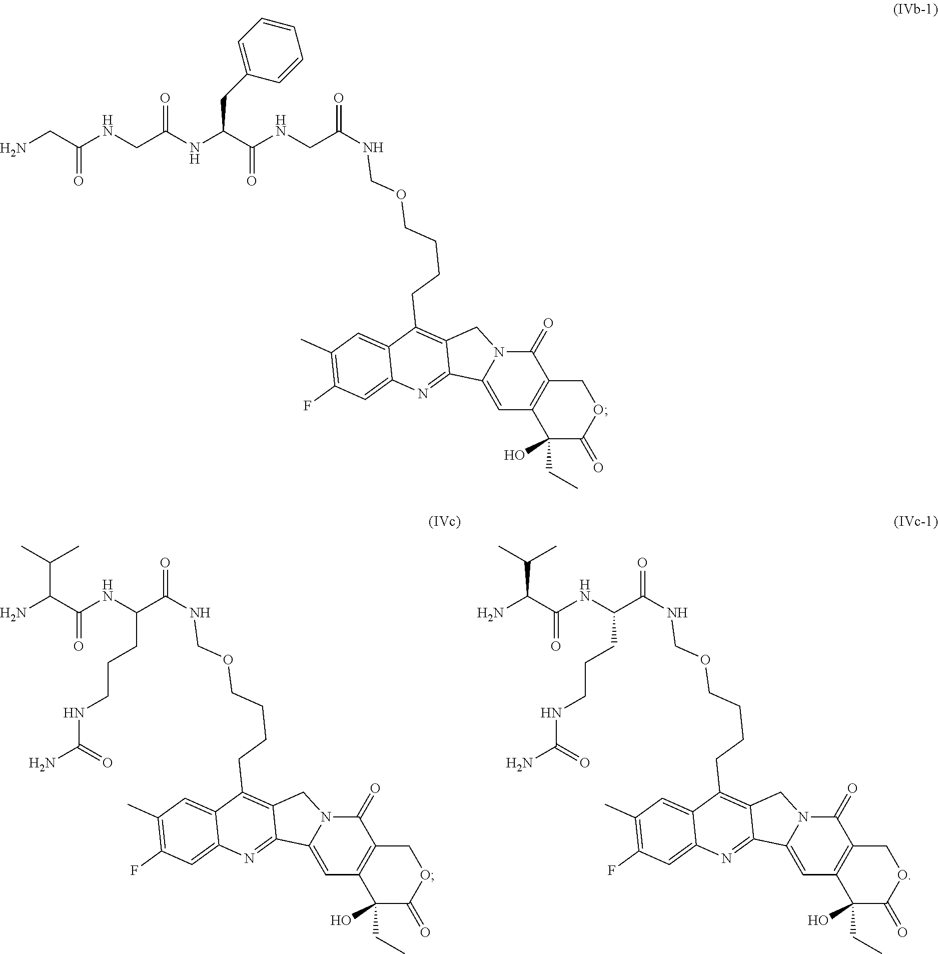 [SMILES: CC[C@@]1(O)C(=O)OCc2c1cc1n(c2=O)Cc2c-1nc1cc(F)c(C)cc1c2CCCCOCNC(=O)CNC(=O)[C@H](Cc1ccccc1)NC(=O)CNC(=O)CN ; CC[C@@]1(O)C(=O)OCc2c1cc1n(c2=O)Cc2c-1nc1cc(F)c(C)cc1c2CCCCOCNC(=O)C(CCCNC(N)=O)NC(=O)C(N)C(C)C ; CC[C@@]1(O)C(=O)OCc2c1cc1n(c2=O)Cc2c-1nc1cc(F)c(C)cc1c2CCCCOCNC(=O)[C@H](CCCNC(N)=O)NC(=O)[C@@H](N)C(C)C]

Any suitable bases can be used for the reaction with the compound of Formula (III). (IIIa), (IIIa-1), (IIIa-2), (IIIb), (IIIb-1), (IIIc) or (IIIc-1) to form the compound of Formula (IV), (IVa), (IVa-1), (IVa-2), (IVb), (IVb-1), (IVc) or (IVc-1), respectively. In some embodiments, the bases are selected from piperidine, morpholine, N-methylmorpholine, 4-methylpiperidine, piperazine, pyrrolidine, 1,8-diazabicyclo[5.4.0]undec-7-ene (DBU), diisopropylethyalamine (DIPEA), triethylamine (TEA), diethylamine (DEA), and a combination thereof. In some embodiments, the base is triethylamine (TEA), piperidine, morpholine, or a combination thereof.

In an eleventh embodiment, the compound of Formula (IV) in the ninth or tenth embodiment is reacted with a compound of Formula (B):

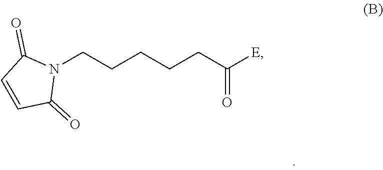

to form a compound of Formula (V):

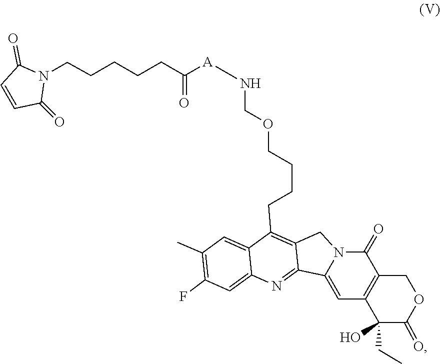

wherein E is —OH, —Cl or

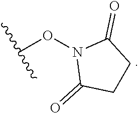 [SMILES: *ON1C(=O)CCC1=O]

In a twelfth embodiment, E is

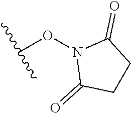 [SMILES: *ON1C(=O)CCC1=O]

and the reaction between the compound of Formula (IV) and the compound of Formula (B) in the eleventh embodiment is carried out in the presence of a base.

Any suitable bases can be used for the reaction between the compound of Formula (IV) and the compound of Formula (B) in the twelfth embodiment. In some embodiments, the base is N-methylmorpholine, triethylamine (TEA), 1,8-diazabicyclo[5.4.0]undec-7-ene (DBU), N-methylpiperidine, 1-methylpyrrolidine, or diisopropylethyalamine (DIPEA). In some embodiments, the base is N-methylmorpholine.

Any suitable solvents can be used for the reaction between the compound of Formula (IV) and the compound of Formula (B) in the twelfth embodiment. In some embodiments, the solvent is dimethylformamide (DMF).

In a thirteenth embodiment, E is —OH and the reaction between the compound of Formula (IV) and the compound of Formula (B) in the eleventh embodiment is carried out in the presence of an activating agent.

In some embodiments, the activating agent used for the reaction between the compound of Formula (IV) and the compound of Formula (B) in the thirteenth embodiment is selected from 2,4,6-trialkyl-1,3,5,2,4,6-trioxatriphosphorinane 2,4,6-trioxide, carbodiimide (e.g., N,N′-dicyclohexylcarbodiimide (DCC) or 1-ethyl-3-(3-dimethylaminopropyl) carbodiimide (EDC)), 1,1′-carbonyldiimidazole (CDI), a uronium, an activated ester, a phosphonium, 2-alkyl-1-alkylcarbonyl-1,2-dihydroquinoline, 2-alkoxy-1-alkoxycarbonyl-1,2-dihydroquinoline, and alkylchloroformate. In some embodiments, the activating agent is 2,4,6-tripropyl-1,3,5,2,4,6-trioxatriphosphorinane 2,4,6-trioxide (T3P).

In some embodiments, the reaction between the compound of Formula (IV) and the compound of Formula (B) in the thirteenth embodiment is carried out in the presence of 1-ethyl-3-(3-dimethylaminopropyl) carbodiimide (EDC) and hydroxybenzotriazole (HOBt).

In a fourteenth embodiment, the compound of Formula (IV) in the eleventh, twelfth, or thirteenth embodiment is represented by Formula (IVa), (IVa-1), (IVa-2), (IVb), (IVb-1), (IVc) or (IVc-1) and the compound of Formula (V) in the eleventh, twelfth, or thirteenth embodiment is represented by Formula (Va), (Va-1), (Va-2), (Vb), (Vb-1), (Vc), or (Vc-1), respectively:

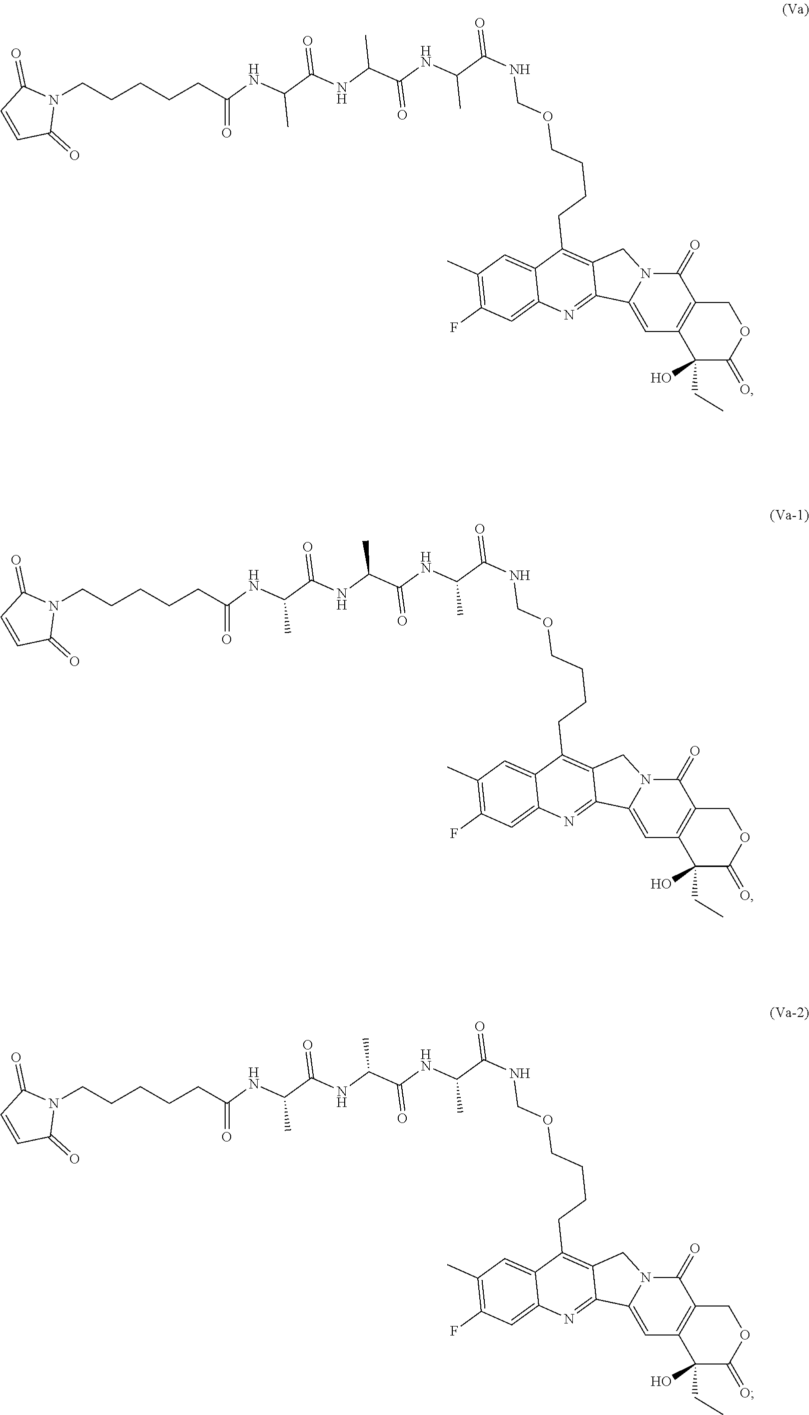 [SMILES: CC[C@@]1(O)C(=O)OCc2c1cc1n(c2=O)Cc2c-1nc1cc(F)c(C)cc1c2CCCCOCNC(=O)C(C)NC(=O)C(C)NC(=O)C(C)NC(=O)CCCCCN1C(=O)C=CC1=O ; CC[C@@]1(O)C(=O)OCc2c1cc1n(c2=O)Cc2c-1nc1cc(F)c(C)cc1c2CCCCOCNC(=O)[C@H](C)NC(=O)[C@H](C)NC(=O)[C@H](C)NC(=O)CCCCCN1C(=O)C=CC1=O ; CC[C@@]1(O)C(=O)OCc2c1cc1n(c2=O)Cc2c-1nc1cc(F)c(C)cc1c2CCCCOCNC(=O)[C@H](C)NC(=O)[C@@H](C)NC(=O)[C@H](C)NC(=O)CCCCCN1C(=O)C=CC1=O]

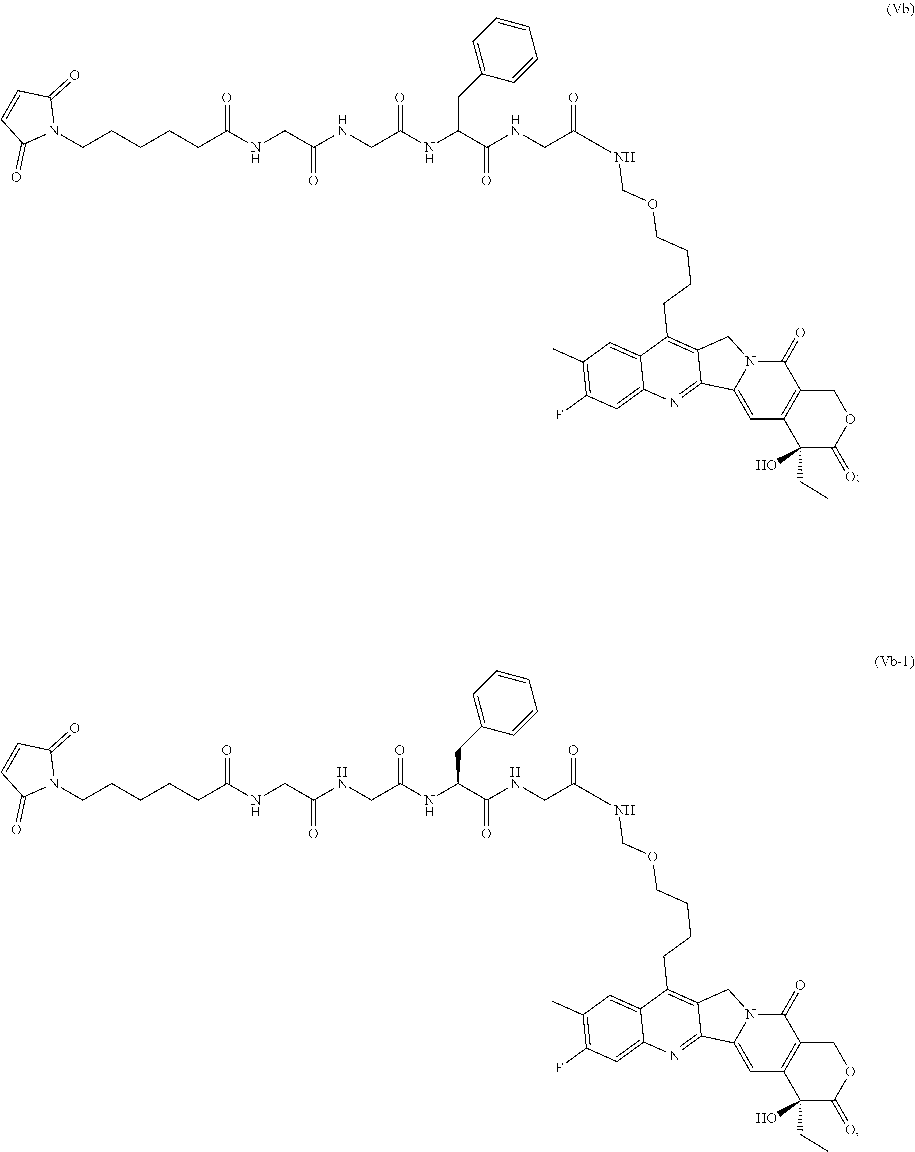 [SMILES: CC[C@@]1(O)C(=O)OCc2c1cc1n(c2=O)Cc2c-1nc1cc(F)c(C)cc1c2CCCCOCNC(=O)CNC(=O)C(Cc1ccccc1)NC(=O)CNC(=O)CNC(=O)CCCCCN1C(=O)C=CC1=O ; CC[C@@]1(O)C(=O)OCc2c1cc1n(c2=O)Cc2c-1nc1cc(F)c(C)cc1c2CCCCOCNC(=O)CNC(=O)[C@H](Cc1ccccc1)NC(=O)CNC(=O)CNC(=O)CCCCCN1C(=O)C=CC1=O]

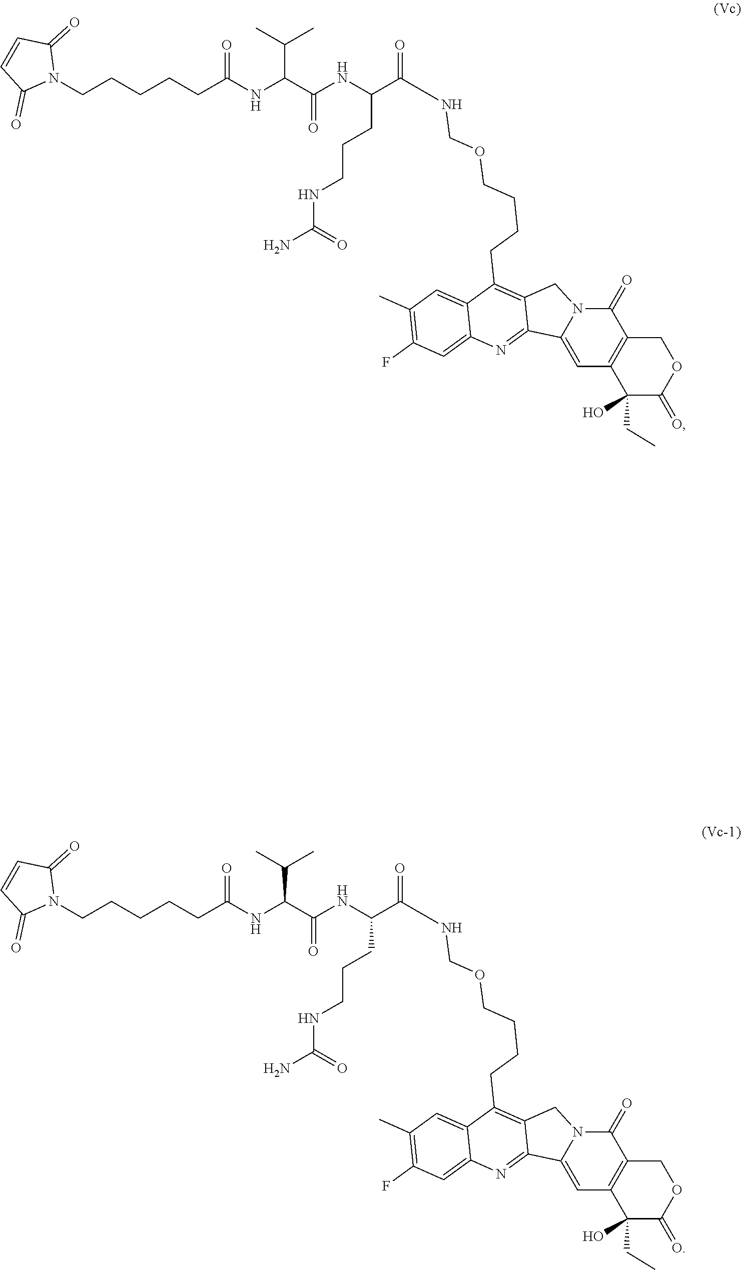 [SMILES: CC[C@@]1(O)C(=O)OCc2c1cc1n(c2=O)Cc2c-1nc1cc(F)c(C)cc1c2CCCCOCNC(=O)C(CCCNC(N)=O)NC(=O)C(NC(=O)CCCCCN1C(=O)C=CC1=O)C(C)C ; CC[C@@]1(O)C(=O)OCc2c1cc1n(c2=O)Cc2c-1nc1cc(F)c(C)cc1c2CCCCOCNC(=O)[C@H](CCCNC(N)=O)NC(=O)[C@@H](NC(=O)CCCCCN1C(=O)C=CC1=O)C(C)C]

In a fifteenth embodiment, the present invention provides a method of preparing a compound of Formula (VII):

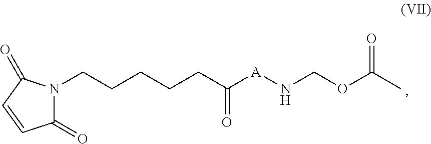

comprising the steps of:
(a) deprotecting a compound of Formula (A):

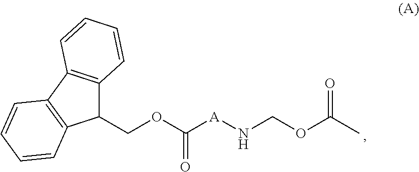

with a base, or with H₂ and a palladium catalyst, to form a compound of Formula (VI):

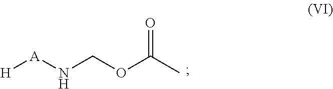

(b) reacting the compound of Formula (VI) with a compound of Formula (B):

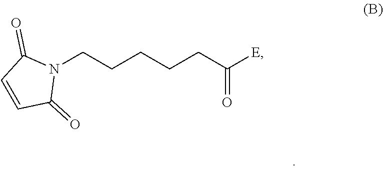

wherein A is a peptide comprising 2 to 10 amino acids; and E is —OH, —Cl or

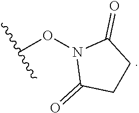 [SMILES: *ON1C(=O)CCC1=O]

Any suitable bases can be used in step (a) of the fifteenth embodiment for deprotecting the compound of Formula (A) to form the compound of Formula (VI). In some embodiments, the base is selected from piperidine, morpholine, N-methylmorpholine, 4-methylpiperidine, piperazine, pyrrolidine, 1,8-diazabicyclo[5.4.0]undec-7-ene (DBU), diisopropylethyalamine (DIPEA), triethylamine (TEA), diethylamine (DEA), and a combination thereof. In some embodiments, the base is triethylamine (TEA), piperidine, diisopropylethyalamine (DIPEA), or a combination thereof.

In some embodiments, the deprotection reaction is carried out by reacting the compound of Formula (A) with H₂ in the presence of a palladium catalyst to form the compound of Formula (VI). Any suitable palladium catalyst can be used. In some embodiments, the palladium catalyst is Pd/C.

Any suitable solvents can be used in step (a) of the fifteenth embodiment. In some embodiments, the solvent is dimethylformamide (DMF), dimethyl sulfoxide (DMSO), or dimethylacetamide (DMA or DMAc).

In a sixteenth embodiment, E is

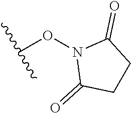 [SMILES: *ON1C(=O)CCC1=O]

and the reaction between the compound of Formula (VI) and the compound of Formula (B) in step (b) of the fifteenth embodiment is carried out in the presence of a base.

Any suitable bases can be used in step (b) of the sixteenth embodiment for the reaction between the compound of Formula (VI) and the compound of Formula (B). In some embodiments, the base in step (b) is N-methylmorpholine, triethylamine (TEA), 1,8-diazabicyclo[5.4.0]undec-7-ene (DBU), N-methylpiperidine, 1-methylpyrrolidine, or diisopropylethyalamine (DIPEA).

In a seventeenth embodiment, E is —OH and the reaction between the compound of Formula (VI) and the compound of Formula (B) in step (b) of the fifteenth embodiment is carried out in the presence of an activating agent.

In some embodiments, the activating agent used for the reaction between the compound of Formula (VI) and the compound of Formula (B) in step (b) of the seventeenth embodiment is selected from 2,4,6-trialkyl-1,3,5,2,4,6-trioxatriphosphorinane 2,4,6-trioxide, carbodiimide (e.g., N,N′-dicyclohexylcarbodiimide (DCC) or 1-ethyl-3-(3-dimethylaminopropyl) carbodiimide (EDC)), 1,1′-carbonyldiimidazole (CDI), a uronium, an activated ester, a phosphonium, 2-alkyl-1-alkylcarbonyl-1,2-dihydroquinoline, 2-alkoxy-1-alkoxycarbonyl-1,2-dihydroquinoline, or alkylchloroformate. In some embodiments, the activating agent is 2,4,6-tripropyl-1,3,5,2,4,6-trioxatriphosphorinane 2,4,6-trioxide (T3P).

In some embodiments, the reaction between the compound of Formula (VI) and the compound of Formula (B) in step (b) of the seventeenth embodiment is carried out in the presence of 1-ethyl-3-(3-dimethylaminopropyl) carbodiimide (EDC) and hydroxybenzotriazole (HOBt).

In some embodiments, the peptide represented by A in the fifteenth, sixteenth, or seventeenth embodiment is a peptide comprising 2 to 4 amino acids. In some embodiments, A is -Ala-Ala-*, -Ala-Val-*, -Val-Ala-*, -Val-Cit-*, -Cit-Val-*, -Gln-Leu-*, -Leu-Gln-*, -Ala-Ala-Ala-*, -Ala-Ala-Ala-Ala-*, -Gly-Ala-Gly-Gly-*, -Gly-Gly-Ala-Gly-*, -Gly-Val-Gly-Gly-*, -Gly-Gly-Val-Gly-*, -Gly-Phe-Gly-Gly-*, or - Gly-Gly-Phe-Gly-*, wherein * is the point of attachment to the carbonyl (—C(═O)—) group in Formula (A). In some embodiments, A is -Val-Cit-*, -Cit-Val-*, -Ala-Ala-Ala-*, -Gly-Phe-Gly-Gly-* or -Gly-Gly-Phe-Gly-*.

In an eighteenth embodiment, the compound of Formula (A) in the fifteenth, sixteenth, or seventeenth embodiment is represented by Formula (Aa); the compound of Formula (VI) in the fifteenth, sixteenth, or seventeenth embodiment is represented by Formula (VIa) and the compound of Formula (VII) in the fifteenth, sixteenth, or seventeenth embodiment is represented by Formula (VIIa):

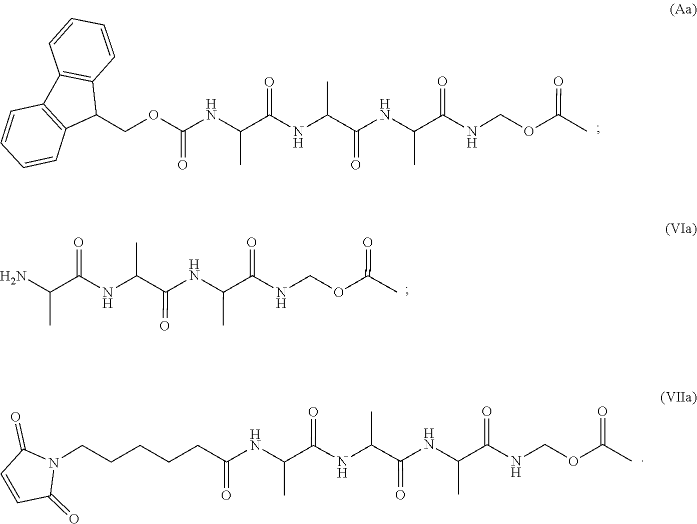 [SMILES: CC(=O)OCNC(=O)C(C)NC(=O)C(C)NC(=O)C(C)NC(=O)OCC1c2ccccc2-c2ccccc21 ; CC(=O)OCNC(=O)C(C)NC(=O)C(C)NC(=O)C(C)N ; CC(=O)OCNC(=O)C(C)NC(=O)C(C)NC(=O)C(C)NC(=O)CCCCCN1C(=O)C=CC1=O]

In a 5^{th} specific embodiment, the compound of Formula (Aa) in the eighteenth embodiment is represented by Formula (Aa-1); the compound of Formula (VIa) in the eighteenth embodiment is represented by Formula (VIa-1) and the compound of Formula (VIIa) in the eighteenth embodiment is represented by Formula (VIIa-1):

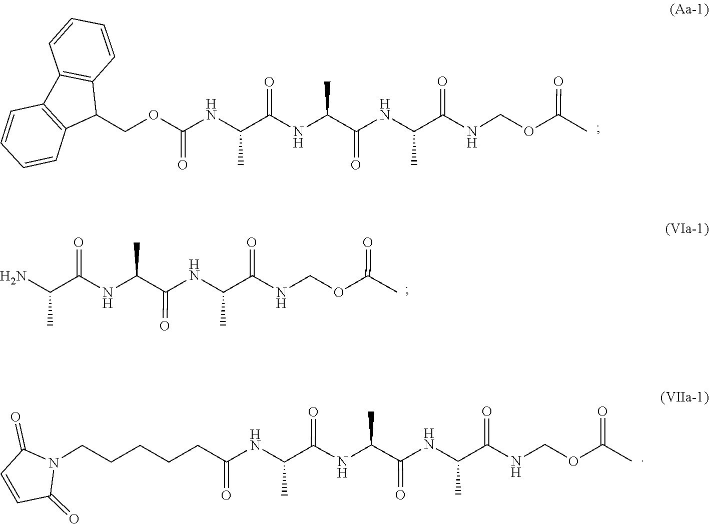 [SMILES: CC(=O)OCNC(=O)[C@H](C)NC(=O)[C@H](C)NC(=O)[C@H](C)NC(=O)OCC1c2ccccc2-c2ccccc21 ; CC(=O)OCNC(=O)[C@H](C)NC(=O)[C@H](C)NC(=O)[C@H](C)N ; CC(=O)OCNC(=O)[C@H](C)NC(=O)[C@H](C)NC(=O)[C@H](C)NC(=O)CCCCCN1C(=O)C=CC1=O]

In a 6^{th} specific embodiment, the compound of Formula (Aa) in the eighteenth embodiment is represented by Formula (Aa-2); the compound of Formula (VIa) in the eighteenth embodiment is represented by Formula (VIa-2) and the compound of Formula (VIIa) in the eighteenth embodiment is represented by Formula (VIIa-2):

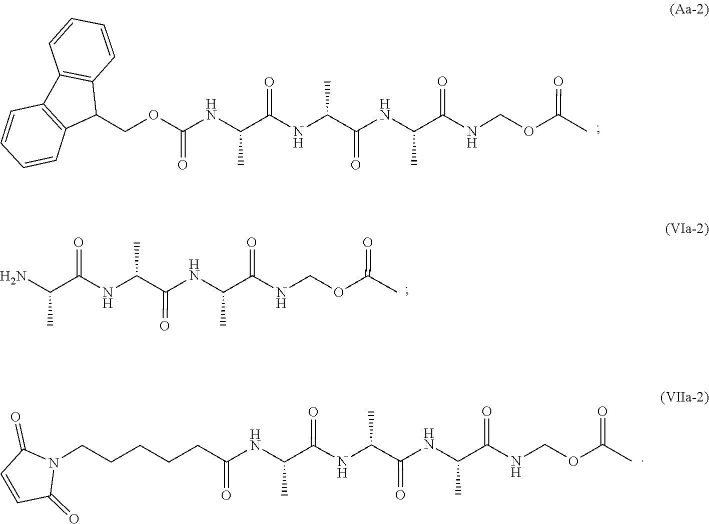 [SMILES: CC(=O)OCNC(=O)[C@H](C)NC(=O)[C@@H](C)NC(=O)[C@H](C)NC(=O)OCC1c2ccccc2-c2ccccc21 ; CC(=O)OCNC(=O)[C@H](C)NC(=O)[C@@H](C)NC(=O)[C@H](C)N ; CC(=O)OCNC(=O)[C@H](C)NC(=O)[C@@H](C)NC(=O)[C@H](C)NC(=O)CCCCCN1C(=O)C=CC1=O]

In a nineteenth embodiment, the compound of Formula (A) in the fifteenth, sixteenth, or seventeenth embodiment is represented by Formula (Ab); the compound of Formula (VI) in the fifteenth, sixteenth, or seventeenth embodiment is represented by Formula (VIb) and the compound of Formula (VII) in the fifteenth, sixteenth, or seventeenth embodiment is represented by Formula (VIIb):

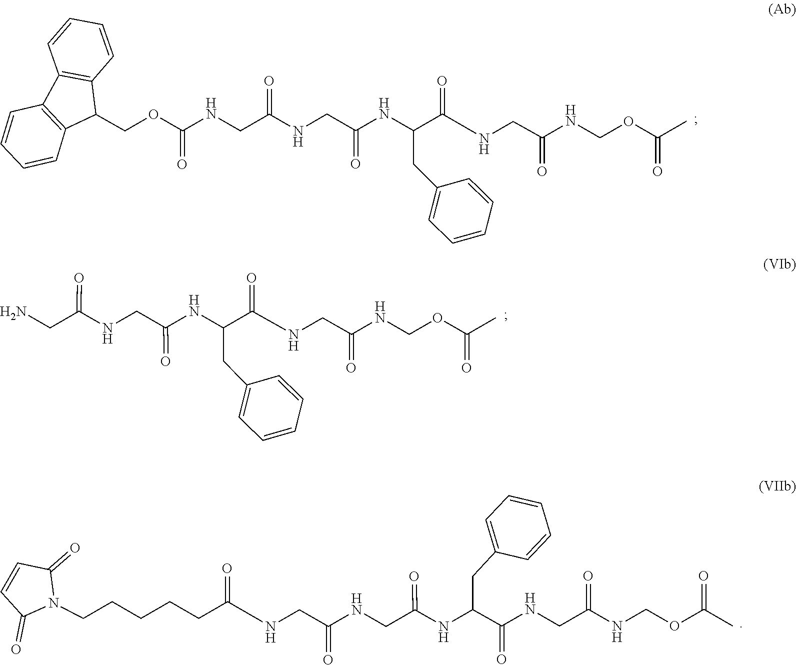 [SMILES: CC(=O)OCNC(=O)CNC(=O)C(Cc1ccccc1)NC(=O)CNC(=O)CNC(=O)OCC1c2ccccc2-c2ccccc21 ; CC(=O)OCNC(=O)CNC(=O)C(Cc1ccccc1)NC(=O)CNC(=O)CN ; CC(=O)OCNC(=O)CNC(=O)C(Cc1ccccc1)NC(=O)CNC(=O)CNC(=O)CCCCCN1C(=O)C=CC1=O]

In a 7^{th} specific embodiment, the compound of Formula (Ab) in the nineteenth embodiment is represented by Formula (Ab-1); the compound of Formula (VIb) in the nineteenth embodiment is represented by Formula (VIb-1) and the compound of Formula (VIIb) in the nineteenth embodiment is represented by Formula (VIIb-1):

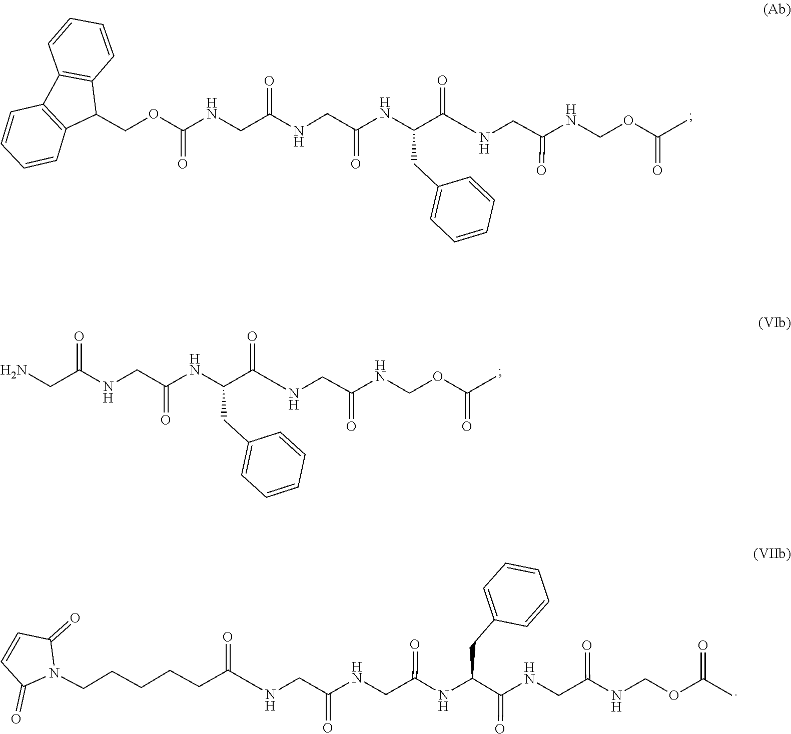 [SMILES: CC(=O)OCNC(=O)CNC(=O)[C@H](Cc1ccccc1)NC(=O)CNC(=O)CNC(=O)OCC1c2ccccc2-c2ccccc21 ; CC(=O)OCNC(=O)CNC(=O)[C@H](Cc1ccccc1)NC(=O)CNC(=O)CN ; CC(=O)OCNC(=O)CNC(=O)[C@H](Cc1ccccc1)NC(=O)CNC(=O)CNC(=O)CCCCCN1C(=O)C=CC1=O]

In a twentieth embodiment, the compound of Formula (A) in the fifteenth, sixteenth, or seventeenth embodiment is represented by Formula (Ac); the compound of Formula (VI) in the fifteenth, sixteenth, or seventeenth embodiment is represented by Formula (VIc) and the compound of Formula (VII) in the fifteenth, sixteenth, or seventeenth embodiment is represented by Formula (VIIc):

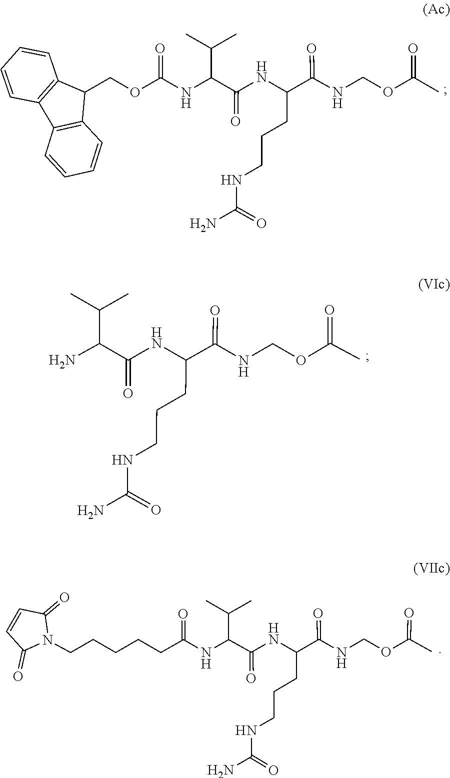 [SMILES: CC(=O)OCNC(=O)C(CCCNC(N)=O)NC(=O)C(NC(=O)OCC1c2ccccc2-c2ccccc21)C(C)C ; CC(=O)OCNC(=O)C(CCCNC(N)=O)NC(=O)C(N)C(C)C ; CC(=O)OCNC(=O)C(CCCNC(N)=O)NC(=O)C(NC(=O)CCCCCN1C(=O)C=CC1=O)C(C)C]

In a 8^{th} specific embodiment, the compound of Formula (Ac) in the twentieth embodiment is represented by Formula (Ac-1); the compound of Formula (VIc) in the twentieth embodiment is represented by Formula (VIc-1) and the compound of Formula (VIIc) in the twentieth embodiment is represented by Formula (VIIc-1):

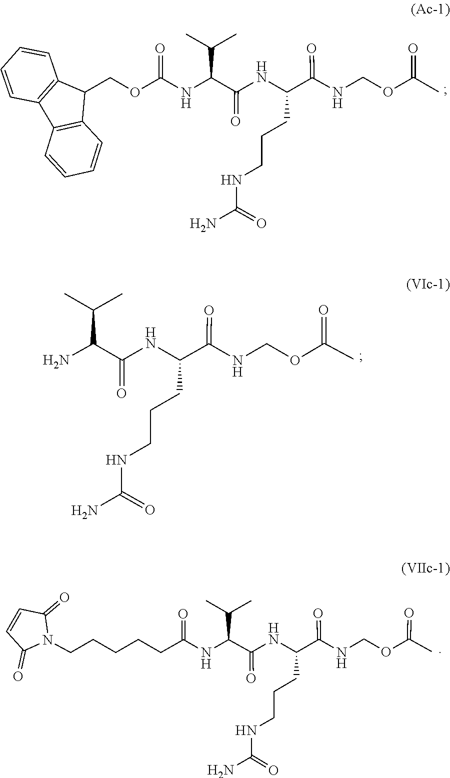

In a twenty-first embodiment, the present invention provides a method of preparing a compound of Formula (V):

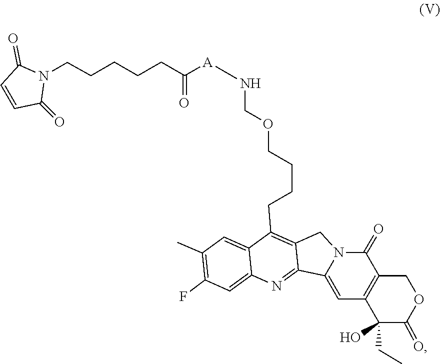

comprising reacting a compound of Formula (VII):

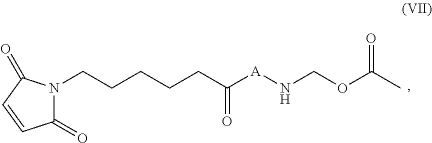

with a compound of Formula (II):

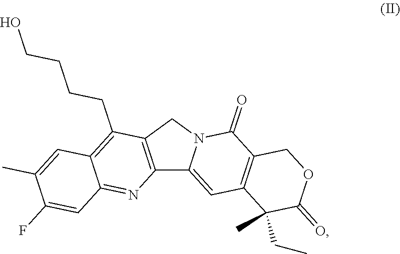 [SMILES: CC[C@@]1(C)C(=O)OCc2c1cc1n(c2=O)Cc2c-1nc1cc(F)c(C)cc1c2CCCCO]

to form the compound of Formula (V).

In a twenty-second embodiment, the reaction between the compound of Formula (VII) and the compound of Formula (II) in the twenty-first embodiment is carried out in the presence of an acid.

Any suitable acids can be used for the reaction between the compound of Formula (VII) and the compound of Formula (II) in the twenty-second embodiment. In some embodiments, the acid is selected from TFA and HCl. In some embodiments, the acid is a Lewis acid. In some embodiments, the acid is a Lewis acid selected from boron trifluoride etherate (BF₃·OEt₂), boron trichloride (BCl₃), and aluminum trichloride (AlCl₃). In some embodiments, the reaction between the compound of Formula (VII) and the compound of Formula (II) in the twenty-second embodiment is carried out in the presence of boron trifluoride etherate (BF₃·OEt₂).

Any suitable solvents can be used for the reaction in the twenty-second embodiment. In some embodiments, the solvent is dimethylformamide (DMF) or dimethyl sulfoxide (DMSO). In some embodiments, the reaction between the compound of Formula (VII) and the compound of Formula (II) in the twenty-second embodiment is carried out in the presence of TFA and the solvent is dimethylformamide (DMF). In some embodiments, the reaction between the compound of Formula (VII) and the compound of Formula (II) in the twenty-second embodiment is carried out in the presence of boron trifluoride etherate (BF₃·OEt₂) and the solvent is dimethyl sulfoxide (DMSO).

In a twenty-third embodiment, the compound of Formula (VII) in the twenty-first or twenty-second embodiment is represented by Formula (VIIa) and the compound of Formula (V) is represented by Formula (Va):

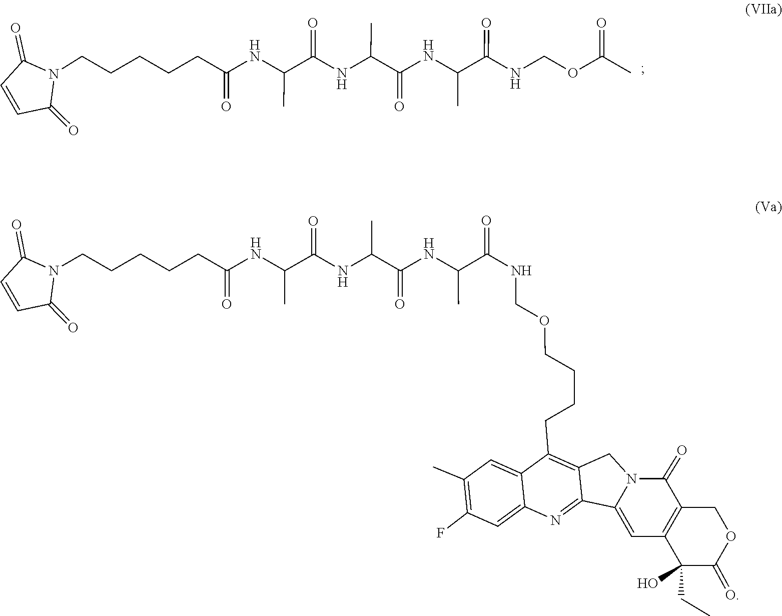 [SMILES: CC(=O)OCNC(=O)C(C)NC(=O)C(C)NC(=O)C(C)NC(=O)CCCCCN1C(=O)C=CC1=O ; CC[C@@]1(O)C(=O)OCc2c1cc1n(c2=O)Cc2c-1nc1cc(F)c(C)cc1c2CCCCOCNC(=O)C(C)NC(=O)C(C)NC(=O)C(C)NC(=O)CCCCCN1C(=O)C=CC1=O]

In a 9^{th} specific embodiment, the compound of Formula (VII) in the twenty-third embodiment is represented by Formula (VIIa-1) and the compound of Formula (V) in the twenty-third embodiment is represented by Formula (Va-1):

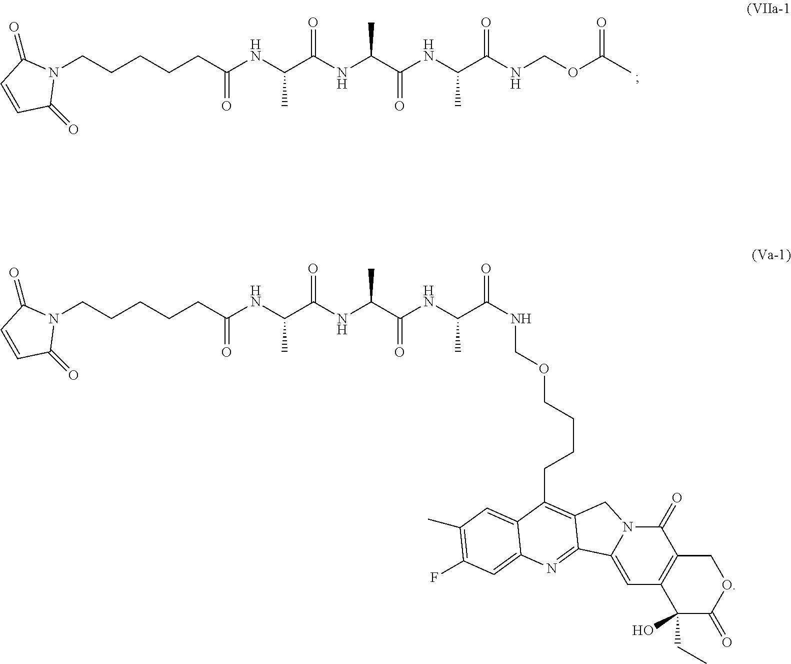 [SMILES: CC(=O)OCNC(=O)[C@H](C)NC(=O)[C@H](C)NC(=O)[C@H](C)NC(=O)CCCCCN1C(=O)C=CC1=O ; CC[C@@]1(O)C(=O)OCc2c1cc1n(c2=O)Cc2c-1nc1cc(F)c(C)cc1c2CCCCOCNC(=O)[C@H](C)NC(=O)[C@H](C)NC(=O)[C@H](C)NC(=O)CCCCCN1C(=O)C=CC1=O]

In a 10^{th} specific embodiment, the compound of Formula (VII) in the twenty-third embodiment is represented by Formula (VIIa-2) and the compound of Formula (V) in the twenty-third embodiment is represented by Formula (Va-2):

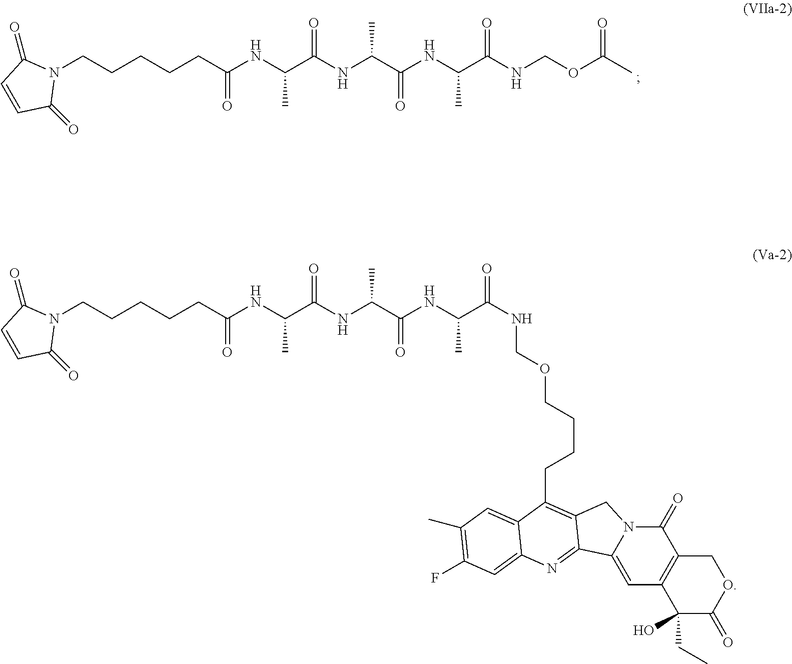 [SMILES: CC(=O)OCNC(=O)[C@H](C)NC(=O)[C@@H](C)NC(=O)[C@H](C)NC(=O)CCCCCN1C(=O)C=CC1=O ; CC[C@@]1(O)C(=O)OCc2c1cc1n(c2=O)Cc2c-1nc1cc(F)c(C)cc1c2CCCCOCNC(=O)[C@H](C)NC(=O)[C@@H](C)NC(=O)[C@H](C)NC(=O)CCCCCN1C(=O)C=CC1=O]

In a twenty-fourth embodiment, the compound of Formula (VII) in the twenty-first or twenty-second embodiment is represented by Formula (VIIb) and the compound of Formula (V) in the twenty-first or twenty-second embodiment is represented by Formula (Vb):

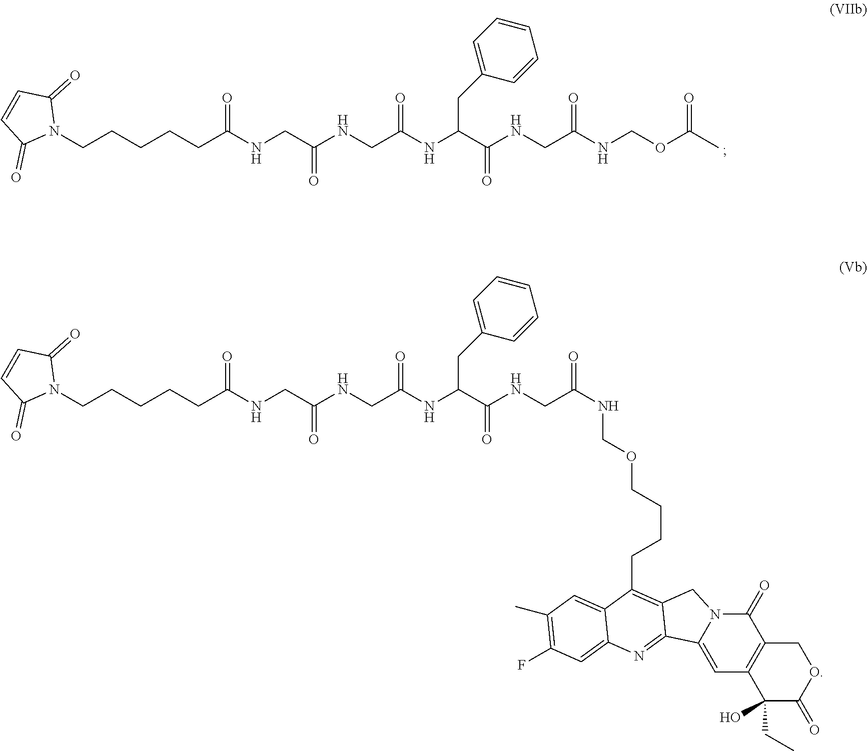 [SMILES: CC(=O)OCNC(=O)CNC(=O)C(Cc1ccccc1)NC(=O)CNC(=O)CNC(=O)CCCCCN1C(=O)C=CC1=O ; CC[C@@]1(O)C(=O)OCc2c1cc1n(c2=O)Cc2c-1nc1cc(F)c(C)cc1c2CCCCOCNC(=O)CNC(=O)C(Cc1ccccc1)NC(=O)CNC(=O)CNC(=O)CCCCCN1C(=O)C=CC1=O]

In a 11^{th} specific embodiment, the compound of Formula (VIIb) in the twenty-fourth embodiment is represented by Formula (VIIb-1) and the compound of Formula (Vb) in the twenty-fourth embodiment is represented by Formula (Vb-1):

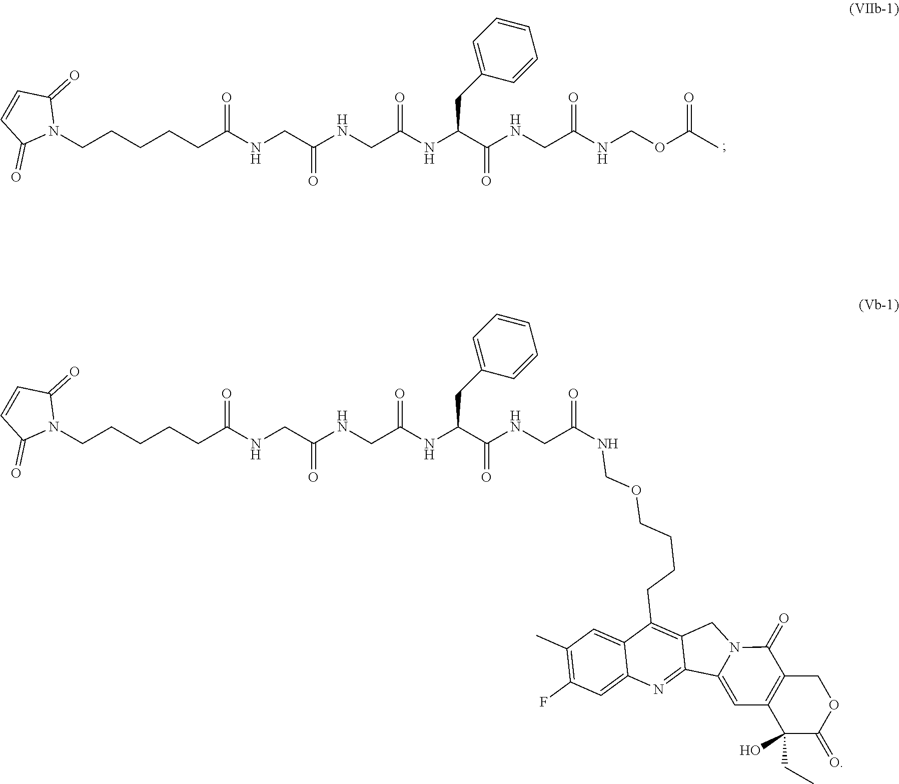 [SMILES: CC(=O)OCNC(=O)CNC(=O)[C@H](Cc1ccccc1)NC(=O)CNC(=O)CNC(=O)CCCCCN1C(=O)C=CC1=O ; CC[C@@]1(O)C(=O)OCc2c1cc1n(c2=O)Cc2c-1nc1cc(F)c(C)cc1c2CCCCOCNC(=O)CNC(=O)[C@H](Cc1ccccc1)NC(=O)CNC(=O)CNC(=O)CCCCCN1C(=O)C=CC1=O]

In a twenty-fifth embodiment, the compound of Formula (VII) in the twenty-first or twenty-second embodiment is represented by Formula (VIIc) and the compound of Formula (V) in the twenty-first or twenty-second embodiment is represented by Formula (Vc):

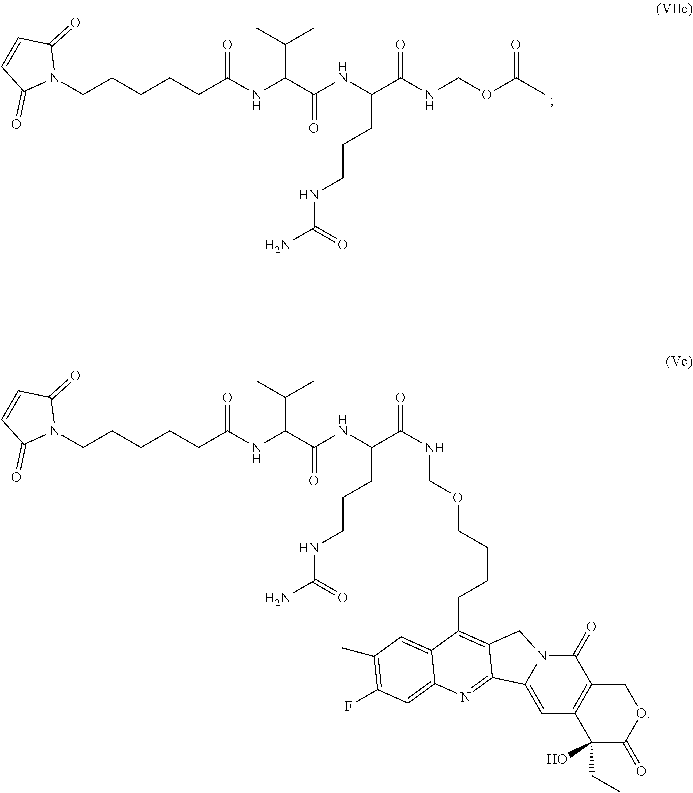 [SMILES: CC(=O)OCNC(=O)C(CCCNC(N)=O)NC(=O)C(NC(=O)CCCCCN1C(=O)C=CC1=O)C(C)C ; CC[C@@]1(O)C(=O)OCc2c1cc1n(c2=O)Cc2c-1nc1cc(F)c(C)cc1c2CCCCOCNC(=O)C(CCCNC(N)=O)NC(=O)C(NC(=O)CCCCCN1C(=O)C=CC1=O)C(C)C]

In a 12^{th} specific embodiment, the compound of Formula (VIIc) in the twenty-fifth embodiment is represented by Formula (VIIc-1) and the compound of Formula (Vc) in the twenty-fifth embodiment is represented by Formula (Vc-1):

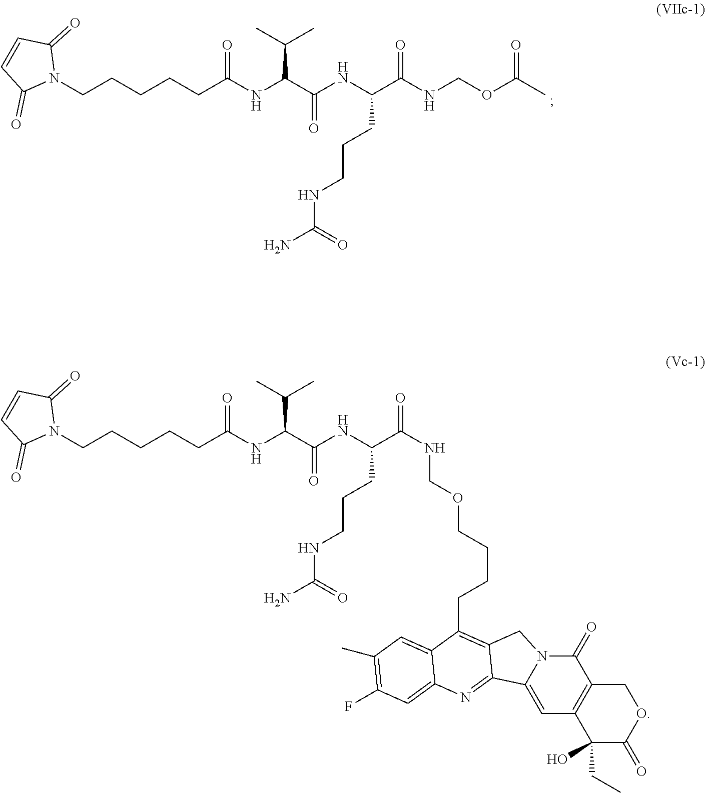

In a twenty-sixth embodiment, the present invention provides a method of preparing a compound of Formula (II):

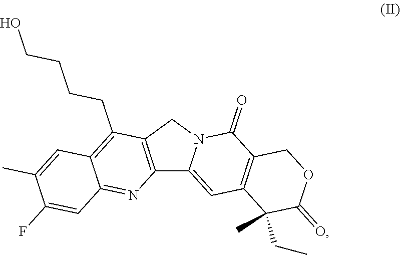 [SMILES: CC[C@@]1(C)C(=O)OCc2c1cc1n(c2=O)Cc2c-1nc1cc(F)c(C)cc1c2CCCCO]

comprising reacting a compound of Formula (VII):

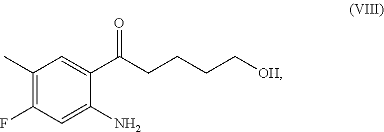 [SMILES: Cc1cc(C(=O)CCCCO)c(N)cc1F]

with a compound of Formula (C):

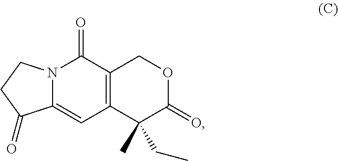 [SMILES: CC[C@@]1(C)C(=O)OCc2c1cc1n(c2=O)CCC1=O]

to form the compound of Formula (II).

In a twenty-seventh embodiment, the reaction between the compound of Formula (VIII) and the compound of Formula (C) in the twenty-sixth embodiment is carried out in the presence of an acid.

Any suitable acids can be used for the reaction between the compound of Formula (VIII) and the compound of Formula (C) in the twenty-seventh embodiment. In some embodiments, the acid is selected from pyridinium p-toluenesulfonate (PPTS), p-toluenesulfonic acid, methanesulfonic acid, camphorsulfonic acid, sulfuric acid, hydrochloric acid, trifluoroacetic acid, and trichloroacetic acid. In some embodiments, the acid is p-toluenesulfonate (PPTS).

Any suitable solvents can be used for the reaction between the compound of Formula (VIII) and the compound of Formula (C) in the twenty-seventh embodiment. In some embodiments, the solvent is toluene.

In a twenty-eighth embodiment, the compound of Formula (VIII) in the twenty-sixth or twenty-seventh embodiment is prepared by reacting a compound of Formula (F)

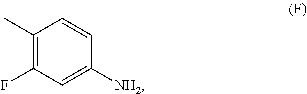

with δ-valerolactone

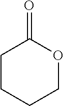

to form the compound of Formula (VIII).

In a twenty-ninth embodiment, the reaction in the twenty-eighth embodiment is carried out in the presence of a Lewis acid catalyst. Any suitable Lewis acid catalysts can be used for the reaction. In some embodiments, the Lewis acid catalyst is selected from AlCl₃, BCl₃, BBr₃, AlBr₃, and GaCl₃. In some embodiments, the Lewis acid catalyst is AlCl₃.

In a thirtieth embodiment, the compound of Formula (VIII) in the twenty-sixth or twenty-seventh embodiment is prepared by a method comprising the following steps:

(a) reacting a compound of formula (G)

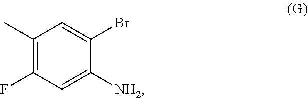

with a compound of (H)

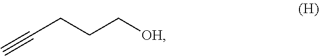

to form a compound of formula (J)

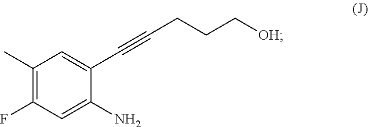

and
(b) reacting the compound of formula (J) with water to form the compound of formula (VIII).

In some embodiments, the reaction in step (a) of the thirtieth embodiment is carried out in the presence of Pd(Ph₃)₂Cl₂ and CuI. In some embodiments, the reaction in step (a) is carried out in the presence of a base. Any suitable base can be used in the reaction of step (a). In some embodiments, the base is triethylamine (TEA). In some embodiments, the reaction in step (a) is carried out in a suitable organic solvent, such as toluene. In some embodiments, the reaction in step (b) is carried out in the presence of an acid, such as H₂SO₄. In some embodiments, the reaction in step (b) is carried out in the presence of H₂SO₄ and HgSO₄.

Any suitable solvent(s) described herein can be used for the reactions described above. Exemplary solvents include dichloromethane (CH₂Cl₂ or DCM), dichloroethane (DCE), acetonitrile (ACN or MeCN), methanol (MeOH), tetrahydrofuran (THF), toluene, N-methylmorpholine (NMM), or any combination thereof. In some embodiments, the solvent is a polar aprotic solvent. Exemplary solvents include, but are not limited to, dimethylformamide (DMF), Dimethyl sulfoxide (DMSO), dimethylacetamide (DMA or DMAc), etc.

### EXAMPLES

### Abbreviations

ACN: Acetonitrile
DCC: N,N′-Dicyclohexylcarbodiimide
DCM: dichloromethane
DCE: dichloroethane
DIEA: N,N-diisopropylethylamine
EA: ethyl acetate
EDC·HCl: 1-(3-dimethylaminopropyl)-3-ethyl-carbodiimide hydrochloride
EDCl: 1-(3-dimethylaminopropyl)-3-ethyl-carbodiimide
FA: Formic acid
HOBT: hydroxybenzotriazole
HOSu: N-hydroxysuccinimide
NHS: N-hydroxysuccinimide
HMPA: Hexamethylphosphoramide
MTBE: Methyl tert-butyl ether
NMM: N-methylmorpholine
NMP: N-methyl-2-pyrollidone
PPTS: Pyridinium p-toluenesulfonate
TLC: thin-layer chromatography
LC-MS: liquid chromatography-mass spectrometry
equiv or eq.: equivalent
L: liter
mL: milliliter
Hrs or h: hour
G: gram

### Example 1. Synthesis of 2,5-dioxopyrrolidin-1-yl 6-(2,5-dioxopyrrol-1-yl)hexanoate (Compound B-1)

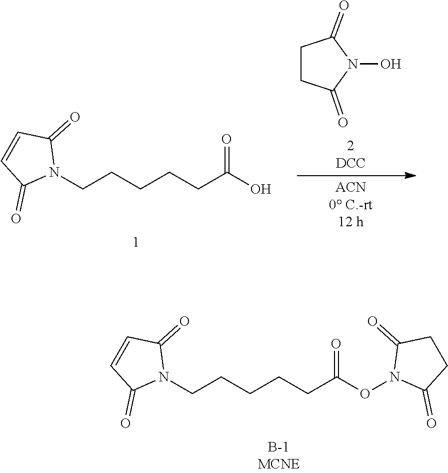 [SMILES: O=C(O)CCCCCN1C(=O)C=CC1=O ; O=C1CCC(=O)N1O ; O=C(CCCCCN1C(=O)C=CC1=O)ON1C(=O)CCC1=O]

A dry 3000 mL round-bottom flask, equipped with a magnetic stirring bar, was charged with N-maleoyl-6-aminohexanoic acid (Compound 1, 120 g, 545.411 mmol) and N-hydroxysuccinimide (Compound 2, 62.77 g,545.411 mmmol) into ACN (1200 mL) under N₂ and cooled to 0° C. DCC (112.54 g,545.411 mmol) was added in portions over and was stirred for 2 h at 0° C. Then the mixture was stirred for 12 h at room temperature. Filtered, the filter cake was washed with ACN. The organic layers were concentrated under reduced pressure to afford 2,5-dioxopyrrolidin-1-yl 6-(2,5-dioxopyrrol-1-yl) hexanoate (Compound B-1, 152 g, 87.6% yield) as a white solid. LC-MS: (ES, m/z): 309 [M+1]⁺. ¹H-NMR: (400 MHz, CDCl₃, ppm): δ 6.703-6.734 (d, 2H), 3.510-3.552 (m, 2H), 2.715-2.840 (s, 4H), 2.586-2.629 (m, 2H), 1.736-1.818 (m, 2H), 1.616-1.651 (m, 2H), 1.391-1.450 (m, 2H).

### Example 2. Synthesis of [(2S)-2-[(2S)-2-[(2S)-2-{[(9H-fluoren-9-ylmethoxy)carbonyl]amino}propanamido] propanamido]propanamido]methyl acetate (Compound Aa-1)

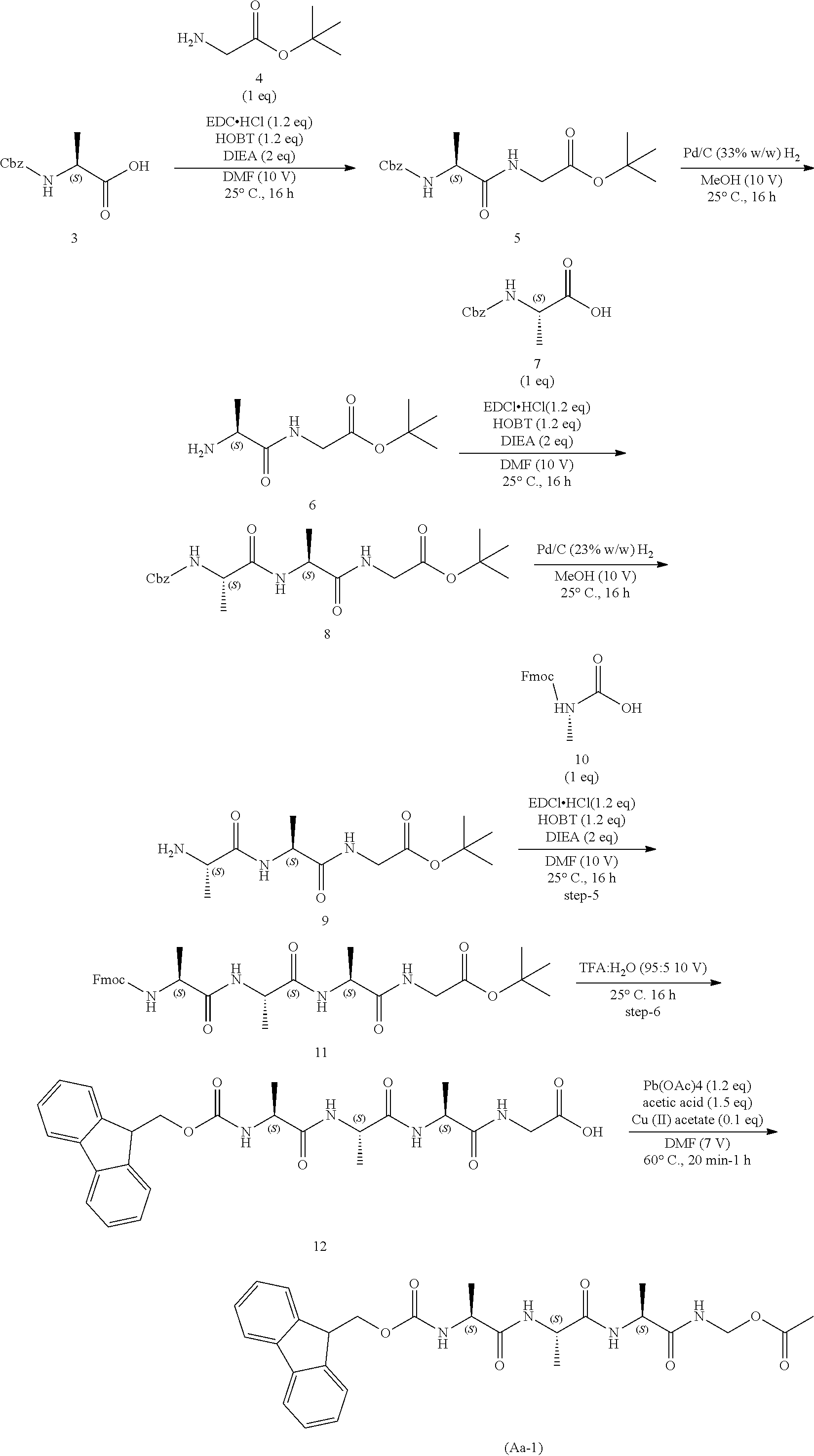

### Step 1. Synthesis of tert-butyl 2-[(2S)-2-{[(benzyloxy)carbonyl]amino}propanamido]acetate (Compound 5)

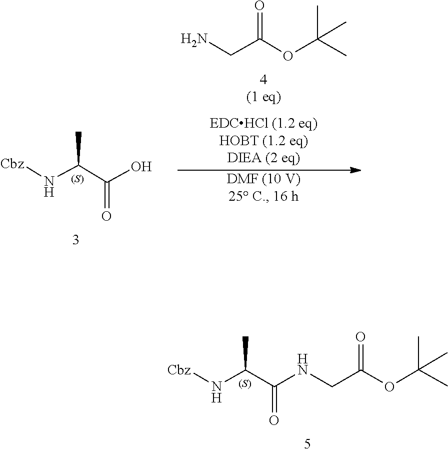

Into a 10 L 4-necked round-bottom flask, was placed (2S)-2-{[(benzyloxy)carbonyl]amino}propanoic acid (Compound 3, 250 g, 1119.931 mmol, 1 equiv), tert-butyl 2-aminoacetate (Compound 4, 146.91 g, 1119.931 mmol, 1 equiv), DMF (2500 mL), 1-(3-dimethylaminopropyl)-3-ethyl-carbodiimide hydrochloride (EDC·HCl, 208.64 g, 1343.917 mmol, 1.2 equiv), hydroxybenzotriazole (HOBT, 181.60 g, 1343.917 mmol, 1.2 equiv), and N,N-diisopropylethylamine (DIEA, 289.50 g, 2239.862 mmol, 2 equiv). The resulting solution was stirred for 16 hrs at 15-25° C. in a water bath. Combined the small scale from 50 g starting material, the reaction was then quenched by the addition of 6 L of water. The resulting solution was extracted with 2×2 L of ethyl acetate and the organic layers combined and dried over anhydrous sodium sulfate and concentrated under vacuum. This resulted in tert-butyl 2-[(2S)-2-{[(benzyloxy)carbonyl]amino}propanamido]acetate (Compound 5, 344 g, 76%) as a yellow solid.

### Step 2. Synthesis of tert-butyl 2-[(2S)-2-aminopropanamido]acetate (Compound 6)

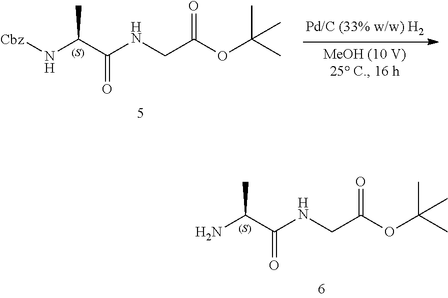

Into a 5 L 3-necked round-bottom flask, was placed tert-butyl 2-[(2S)-2-{[(benzyloxy)carbonyl]amino}propanamido]acetate (Compound 5, 324 g, 963.173 mmol, 1 equiv), Pd/C (96.35 g, 905.383 mmol, 0.94 equiv), MeOH (3239.85 mL, 80020.413 mmol, 83.08 equiv). H₂ gas was then introduced in. The resulting solution was stirred for 12 hr at 25° C. Combined the small scale from 20 g starting material, all the were filtered out and the organic layers concentrated under vacuum to give tert-butyl 2-[(2S)-2-aminopropanamido]acetate (Compound 6, 182 g, 87.99%) as a colorless oil.

### Step 3. Synthesis of tert-butyl 2-[(2S)-2-[(2S)-2-{[(benzyloxy)carbonyl]amino}propanamido]propanamido]acetate (Compound 8)

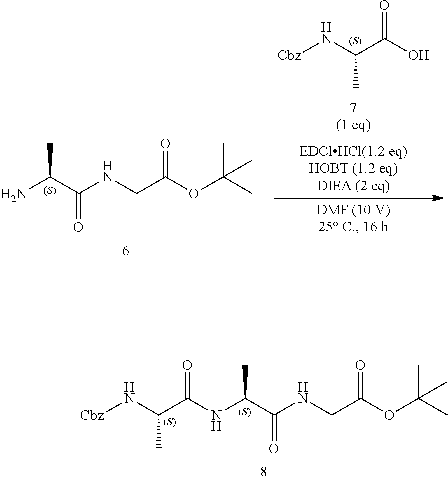

Into a 5 L 4-necked round-bottom flask purged and maintained with an inert atmosphere of nitrogen, was placed tert-butyl 2-[(2S)-2-aminopropanamido]acetate (Compound 6, 162 g, 800.973 mmol, 1 equiv), (2S)-2-{[(benzyloxy)carbonyl]amino}propanoic acid (Compound 7, 178.80 g, 800.973 mmol, 1 equiv), dimethylformamide (1620 mL), EDC·HCl (184.26 g, 961.168 mmol, 1.2 equiv), HOBT (129.88 g, 961.168 mmol, 1.2 equiv), DIEA (207.05 g, 1601.946 mmol, 2 equiv). The resulting solution was stirred for 12 hrs at 25° C. Combined the small scale from 20 g starting material, the reaction was then quenched by the addition of 6 L of water/ice. The resulting solution was extracted with 3×2.4 L of ethyl acetate, the organic phase was dried over anhydrous sodium sulfate and concentrated under vacuum. This is result in tert-butyl 2-[(2S)-2-[(2S)-2-{[(benzyloxy)carbonyl]amino}propanamido]propanamido]acetate (Compound 8, 278 g, 76%) as a white solid.

### Step 4. Synthesis of tert-butyl 2-[(2S)-2-[(2S)-2-aminopropanamido]propanamido]acetate (Compound 9)

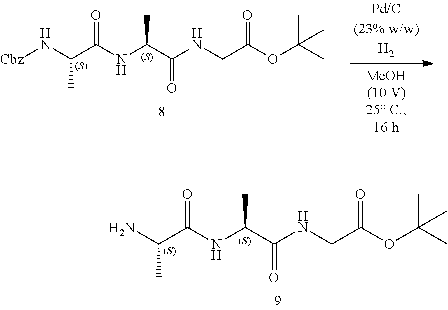

Into a 5 L 3-necked round-bottom flask, was placed tert-butyl 2-[(2S)-2-[(2S)-2-{[(benzyloxy)carbonyl]amino}propanamido]propanamido]acetate (Compound 8, 258 g, 633.180 mmol, 1 equiv), MeOH (2580 mL). H₂ gas was introduced in. The resulting solution was stirred for 12 hr at 25° C. Combined the small scale from 20 g starting material, all the organic layers concentrated under vacuum to give tert-butyl 2-[(2S)-2-[(2S)-2-aminopropanamido]propanamido]acetate (Compound 9, 160 g, 86%) as a colorless oil.

### Step 5. Synthesis of tert-butyl 2-[(2S)-2-[(2S)-2-[(2S)-2-{[(9H-fluoren-9-ylmethoxy)carbonyl]amino}propanamido] propanamido]propanamido]acetate (Compound 11)

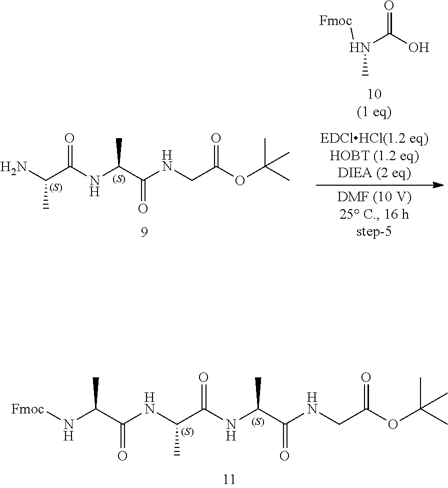

Into a 5 L 3-necked round-bottom flask purged and maintained with an inert atmosphere of nitrogen, was placed tert-butyl 2-[(2S)-2-[(2S)-2-aminopropanamido]propanamido]acetate (Compound 9, 140 g, 512.196 mmol, 1 equiv), DMF (1400 mL), (2S)-2-{[(9H-fluoren-9-ylmethoxy)carbonyl]amino}propanoic acid (Compound 10, 159.47 g, 512.196 mmol, 1 equiv), EDC·HCl (117.83 g, 614.635 mmol, 1.2 equiv), HOBT (83.05 g, 614.635 mmol, 1.2 equiv), DIEA (132.40 g, 1024.392 mmol, 2 equiv). The resulting solution was stirred for 12 hr at 25° C. in a water bath. Combined the small scale from 20 g starting material, the reaction was then quenched by the addition of 6700 ml of water/ice. The resulting solution was extracted with 3×2000 mL of ethyl acetate, the organic phase was dried over anhydrous sodium sulfate and concentrated under vacuum. These resulted in tert-butyl 2-[(2S)-2-[(2S)-2-[(2S)-2-{[(9H-fluoren-9-ylmethoxy)carbonyl]amino}propanamido] propanamido]propanamido]acetate (Compound 11, 248 g, 75%) as off-white solid.

### Step 6. Synthesis of [(2S)-2-[(2S)-2-[(2S)-2-{[(9H-fluoren-9-ylmethoxy)carbonyl]amino}propanamido]propanamido]propanamido]acetic acid (Compound 12)

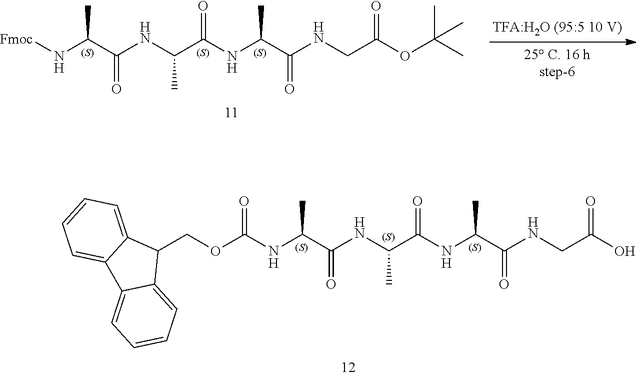

Into a 5 L 3-necked round-bottom flask purged and maintained with an inert atmosphere of nitrogen, was placed tert-butyl 2-[(2S)-2-[(2S)-2-[(2S)-2-{[(9H-fluoren-9-ylmethoxy)carbonyl]amino}propanamido] propanamido]propanamido]acetate (Compound 11, 228 g, 402.361 mmol, 1 equiv), TFA:H₂O (95:5 2280 mL). The resulting solution was stirred for 12 hr at 25° C. Combined the small scale from 20 g starting material, the resulting mixture was concentrated under vacuum. These resulted in [(2S)-2-[(2S)-2-[(2S)-2-{[(9H-fluoren-9-ylmethoxy)carbonyl]amino}propanamido]propanamido]propanamido]acetic acid (Compound 12, 175 g, 78.28%) as a white solid.

### Step 7. Synthesis of [(2S)-2-[(2S)-2-[(2S)-2-{[(9H-fluoren-9-ylmethoxy)carbonyl]amino}propanamido] propanamido]propanamido]methyl acetate (Compound Aa-1)

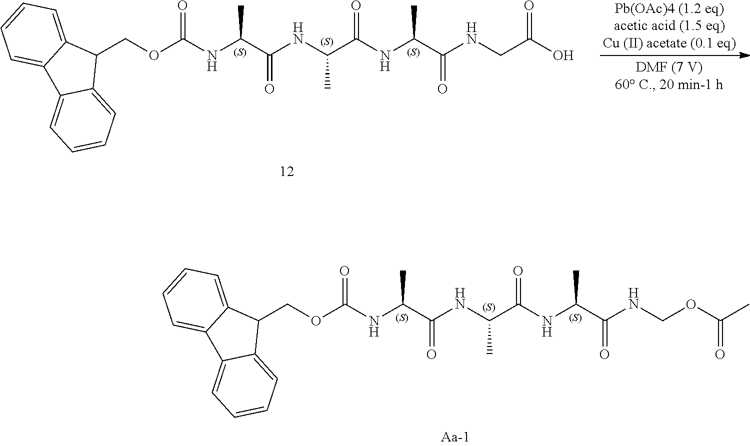

Into a 5 L 4-necked round-bottom flask purged and maintained with an inert atmosphere of nitrogen, was placed [(2S)-2-[(2S)-2-[(2S)-2-{[(9H-fluoren-9-ylmethoxy)carbonyl]amino}propanamido] propanamido]propanamido]acetic acid (Compound 12, 175 g, 342.770 mmol, 1 equiv), lead tetraacetate (Pb(OAc)₄, 151.98 g. 342.770 mmol, 1 equiv), DMF (1750 mL, 22612.696 mmol, 65.97 equiv), Cu (OAc) 2 (6.23 g, 34.277 mmol, 0.1 equiv), acetic acid (30.88 g, 514.155 mmol, 1.5 equiv). The resulting solution was stirred for 20 min at 60° C. The reaction was then quenched by the addition of 3500 mL of water. The resulting solution was extracted with 2×4000 mL of ethyl acetate, the organic phase was concentrated under vacuum. The crude product was purified by Prep-HPLC with the following conditions (IntelFlash-1): Column, C18; mobile phase, A: H₂O; B: CH₃CN (hold 30%-50% ACN in 40 min); Detector, 210 nm. Then the product was purified by silica gel chromatography eluted with DCM:TFE:DMF=5:1:0.06. This resulted in [(2S)-2-[(2S)-2-[(2S)-2-{[(9H-fluoren-9-ylmethoxy)carbonyl]amino}propanamido] propanamido]propanamido]methyl acetate (Compound Aa-1, 53 g, 29.48%) as a white solid. LC-MS: (ES, m/z): 523 [M−H]⁻.

¹H-NMR: (300 MHz, DMSO-d6, ppm): δ 8.87 (t, J=7.0 Hz, 1H), 7.99 (dd, J=12.4, 7.4 Hz, 2H), 7.90 (d, J=7.5 Hz, 2H), 7.78-7.68 (m, 2H), 7.55 (d, J=7.5 Hz, 1H), 7.38 (dtd, J=26.5, 7.4, 1.2 Hz, 4H), 5.17-5.03 (m, 2H), 4.34-4.16 (m, 5H), 4.07 (p, J=7.1 Hz, 1H), 1.99 (s, 3H), 1.21 (dt, J=7.2, 2.6 Hz, 9H).

### Example 3. Synthesis of Compound Va-1

### General Conditions:

Solvents and reagents were purchased from Sigma-Aldrich, VWR, or Fisher Scientific, and used without further purification. Reactions were monitored either by thin-layer chromatography (TLC) or by analytical liquid chromatography-mass spectrometry (LC-MS) employing a Waters Acquity Ultra Performance LC system and a Synapt high-definition mass spectrometer. ¹H NMR spectra were recorded on Bruker (400 MHz) All chemical shifts are reported in ppm and coupling constants, J, are reported in hertz (Hz). NMR solvent peaks were referenced as follows: (1H-NMR) CDCl₃: 7.27 ppm, DMSO-d₆: 2.50 ppm. Compounds were purified by flash column chromatography on a Biotage Isolara or Buchi Reveleris X2 instrument.

### Analytical Methods:

### LC-MS: (HCOOH Method, Long Run)

Mobile phase A: 0.1% Formic acid (FA) in H₂O:ACN (95:5)
Mobile phase: B: Acetonitrile
Flow: 0.8 mL/min
COLUMN: ATLANTIS dC18 (250×4.6) mm, 5 μm

| Time (min) | % B |
|---|---|
| 0 | 5 |
| 15 | 100 |
| 20 | 100 |
| 26 | 5 |
| 30 | 5 |

### LC-MS: (HCOOH Method, Short Run)

A: 0.1% HCOOH IN H₂O:ACN (95:5); B: ACN; Flow Rate: 1.5 mL/min
COLUMN: ZORBAX XDB-C18 (50×4.6 mm) 3.5 μm

| Time (min) | % B |
|---|---|
| 0 | 05 |
| 2.5 | 95 |
| 4.0 | 95 |
| 4.5 | 05 |
| 6.0 | 05 |

### LC-MS: (TFA Method)

A: 0.1% TFA IN H₂O:ACN (95:5); B: 0.1% TFA IN ACN; Flow Rate: 1.5 mL/min
COLUMN: XBridge C8 (50×4.6 mm, 3.5μ),

| Time (min) | % B |
|---|---|
| 0 | 05 |
| 2.5 | 95 |
| 4.5 | 95 |
| 4.5 | 05 |
| 6.0 | 05 |

### HPLC (10 Min Run):

A: 0.1% TFA IN H₂O, B: 0.1% TFA IN ACN; Flow Rate: 2.0 mL/min
Column: X-Bridge C8(50×4.6)mm, 3.5 μm

| Time (min) | % B |
|---|---|
| 0 | 05 |
| 8.0 | 100 |
| 8.1 | 100 |
| 8.5 | 05 |
| 10.0 | 05 |

### HPLC (30 Min Run):

A: 0.1% TFA IN H₂O, B: ACN; Flow Rate: 1.0 mL/min
Column: Atlantis dC18 (250×4.6)mm, 5 μm

| Time (min) | % B |
|---|---|
| 0 | 30 |
| 15 | 100 |
| 20 | 100 |
| 26 | 30 |
| 30 | 30 |

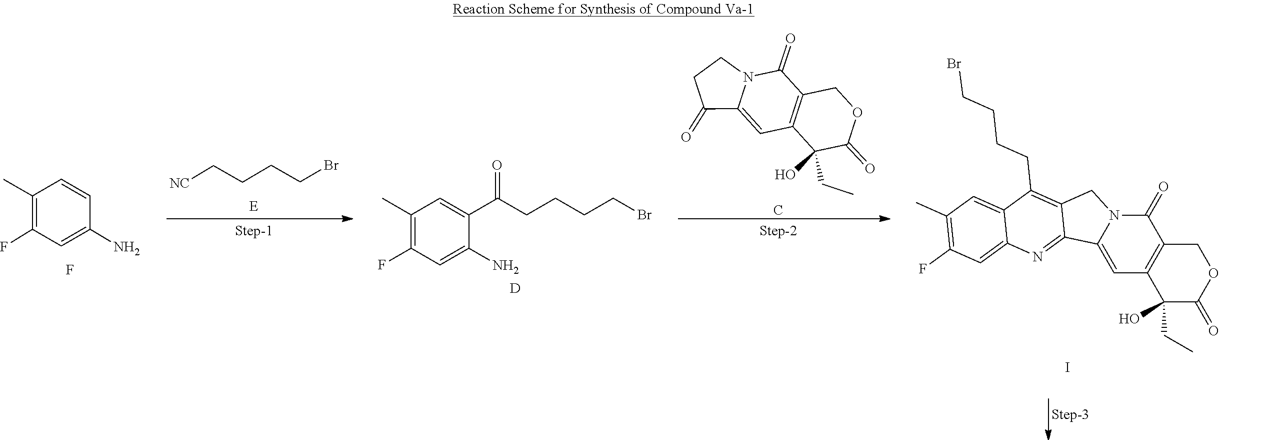

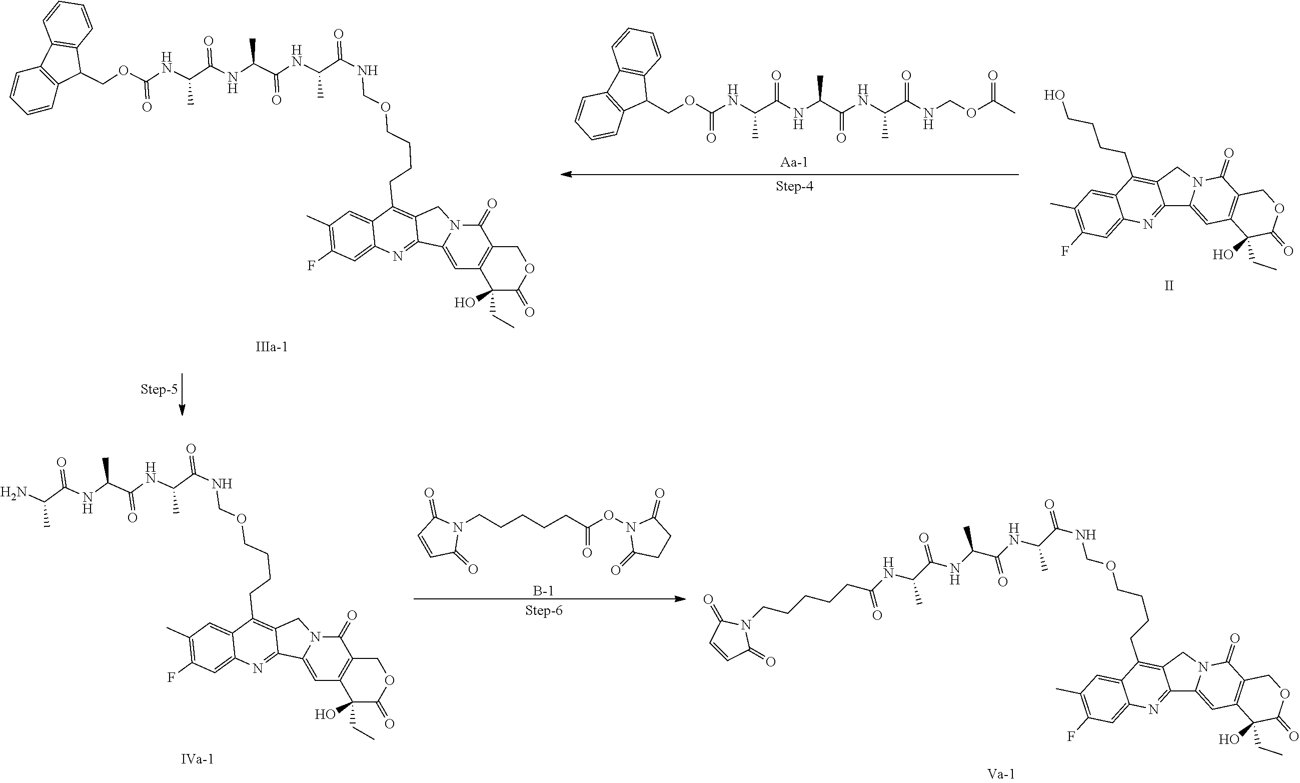

### Step 1. Synthesis of Compound D

To a flask containing anhydrous 1,2-dichloroethane (250 ml) was added 1 M boron trichloride in dichloromethane (Sainor, Catalog Number: BTCL0040621) (132 ml, 127.8 mmol). Cooled to 0° C. with an ice water bath. Compound F (Combi-Blocks, Catalog Number: OR-1810) (20 g, 159.8 mmol) was added in portions and stirred at 0° C. for 10 min. 5-Bromopentanitrile (Combi-Blocks, Catalog Number: QF-7699) (Compound E, 31.06 g, 191.7 mmol) was added to the reaction mixture followed by the addition of aluminium chloride (Sigma-Aldrich, Catalog Number: 237051) (27.8 g, 207.7 mmol). The ice bath was removed, and the reaction solution was gradually warmed to room temperature. After stirring at room temperature for 10 min, the reaction mixture was refluxed for 12 h. The reaction mixture was cooled to room temperature and poured into cold water (350 ml). The crude mixture was extracted with Ethyl acetate (2×350 ml). The combined organic layers were washed with 1.5 N HCl solution, water and brine, dried over anhydrous sodium sulphate and filtered. The filtrate was concentrated under reduced pressure to give Compound D as reddish gum (20 g crude).

LCMS (ESI) m/z calculated for C₁₂H₁₅BrFNO⁺ [M+H]: 288.03; found: 290.1. LCMS: Purity: 91.87%. ¹HNMR (400 MHz, DMSO-d6): δ 7.74 (d, J=8.8 Hz, 1H), 7.25 (s, 2H), 6.48 (d, J=12.4 Hz, 1H), 3.59 (t, J=13.2 Hz, 2H), 2.95 (t, J=14.8 Hz, 2H), 2.11 (s, 3H), 1.90-1.83 (m, 2H), 1.73-1.68 (m, 2H).

### Step 2. Synthesis of Compound I

Compound D (64 g, 189 mmol) and compound C (HaBo Hong Kong Co., Limited.) (11.78 g, 47.25 mmol) in toluene (600 ml) was added PPTS (0.174 g, 0.69 mmol) and refluxed for 16 h in Dean-Stark apparatus. After completion Solvent was evaporated and residue was taken up in a minimum volume of 10% methanol in CH₂Cl₂ then purified by column chromatography (40 g silica 230-400 elute CH₂Cl₂/MeOH, 0 to 10% over 20 min) to give Compound I (10 g). LCMS (ESI): m/z calculated for C₂₅H₂₄BrFN₂O₄⁺ [M+H]: 515.09; found: 515.1. LCMS: Purity: 97.84%. ¹HNMR (400 MHz, DMSO-d6): δ 8.24 (d, J=8.0 Hz, 1H), 7.86 (dd, J=10.8 Hz, 1H), 7.30 (s, 1H), 6.54 (s, 1H), 5.44 (s, 2H), 5.30 (s, 2H), 3.65 (t, J=13.6 Hz, 2H), 3.24-3.17 (m, 2H), 2.51 (t, J=5.2 Hz, 3H), 2.07-2.02 (m, 2H), 2.00-1.79 (m, 4H), 0.89 (t, J=7.20 Hz, 3H).

### Step 3: Synthesis of Compound II

A solution of compound I (13 g, 21.44 mmol) in N-methyl-2-pyrollidone (NMP, 70 mL) and deionized water (35 ml) was heated at 100° C. for 36 h. The reaction mixture was cooled to room temperature and poured into ice cold water (600 ml). The resulting solid was filtered and dried under vacuum to afford crude product. The crude solid was further washed with ice cold water yielded 10 g Compound II as brown solid (87%). 500 mg of Compound II was re-purified by RP flash chromatography obtained 130 mg.

LCMS (ESI): m/z calculated for C₂₅H₂₅FN₂O₅⁺ [M+H]: 453.18; found: 453.2. LCMS: Purity: 94.14% ¹HNMR (400 MHz, DMSO-d6): δ 7.91-7.72 (m, 1H), 7.42-7.31 (m, 1H), 6.53 (s, 1H), 5.44 (s, 2H), 5.30 (s, 2H), 4.45-4.22 (m, 4H), 3.48-3.12 (m, 3H), 2.53 (t, J=14.0 Hz, 3H), 1.89-1.62 (m, 4H), 0.89 (t, J=7.2 Hz, 3H).

### Step 4. Synthesis of Compound IIIa-1

To a stirred solution of Compound II (6.8 g, 15.03 mmol) and Compound Aa-1 (Pharmaron Beijing Co., LTD, (7.88 g, 15.03 mmol) in anhydrous DMF (170 ml) was added TFA (2.44 mL, 30.06 mmol). The reaction mixture allowed to stir for 22 h at room temperature. The reaction solution was concentrated under reduce pressure (35° C. heating bath) to get crude. The crude residue was purified by reverse phase flash chromatography (30 g C18 column eluted with 0.1% FA in CH₃CN/H₂O). The required fractions were extracted with DCM (2×500 ml). Combined organic layers were washed with brine and dried over Na₂SO₄, filtered and concentrated to get Compound IIIa-1 as a brown solid 5.5 g (40.4%). LCMS (ESI): m/z calculated for C₅₀H₅₃FN₆O₁₀⁺ [M+H]: 917.38; found: 917.2. LCMS: Purity: 88.96%. 1H-NMR (400 MHz, DMSO-d6): δ 8.56 (t, J=7.60 Hz, 1H), 8.10 (d, J=11.20 Hz, 2H), 7.98-7.88 (m, 4H), 7.71-7.68 (m, 2H), 7.53 (d, J=9.20 Hz, 1H), 7.42-7.29 (m, 5H), 5.43 (s, 2H), 5.23 (s, 2H) 4.54-4.51 (m, 2H), 4.25-4.16 (m, 5H), 4.05-4.00 (m, 2H), 3.43 (m, 2H), 3.18 (m, 2H), 1.86 (t, J=8.80 Hz, 3H), 1.68 (s, 5H), 1.22-1.16 (m, 10H), 0.87 (t, J=9.60 Hz, 3H).

### Step 5. Synthesis of Compound IVa-1

To a stirred solution of Compound IIIa-1 (3.4 g, 3.71 mmol) in anhydrous DCM (200 ml) was added Piperidine (3.67 ml, 3.71 mmol). The reaction mixture allowed to stir for 1 h at room temperature. The reaction solution was concentrated under reduce pressure (35° C. heating bath) to get crude. The crude residue was washed with diethyl ether, solid was filtered and dried under reduced pressure to get Compound IVa-1 as an off-white solid. The white solid was again purified by reveres phase column chromatography (30 g C18 column eluted with 0.1% FA in CH₃CN/H₂O). The required fractions were lyophilized to get Compound IVa-1 as white solid 1.3 g (39%). LCMS (ESI): m/z calculated for C₃₅H₄₃FN₆O₈⁺ [M+H]: 696.31; found: 695.9.

HPLC: Purity: 98.76% (max).

¹H NMR (400 MHz, DMSO-d6): δ 8.68 (d, J=12.4 Hz, 1H), 8.36-8.29 (m, 3H), 8.16 (d, J=8.4 Hz, 1H), 7.82 (d, J=10.8 Hz, 1H), 5.43 (s, 2H), 5.21 (s, 2H), 4.54 (d, J=14.8 Hz, 2H), 4.31-4.24 (m, 2H), 3.43 (d, J=4.4 Hz, 2H), 3.16 (d, J=9.6 Hz, 2H), 2.49 (t, J=16.80 Hz, 3H), 1.89-1.85 (m, 2H), 1.67 (s, 4H), 1.21 (d, J=16.00 Hz, 9H), 0.89 (t, J=6.80 Hz, 3H).

### Step 6. Synthesis of Compound Va-1

To a solution of Compound IVa-1 (1.4 g, 1.79 mmol) in anhydrous DMF (20 ml) was added Compound B-1 (Combi-Blocks, Catalog Number: QA-0763) (0.67 g, 2.16 mmol) and N-methyl-morpholine (0.57 mL, 5.38 mmol). The reaction mixture was stirred for 2 h at room temperature. The reaction solution was stripped under reduced pressure at 35° C. The residue was purified by reverse phase flash chromatography (330 g C18 column, eluted with 0.1% FA in CH₃CN/H₂O). The required fractions were lyophilized to get Compound Va-1 as white solid 10 g (HPLC purity 83%). The obtained product was re-purified by reverse phase flash chromatography (330 g C18 column, eluted with 0.1% FA in CH₃CN/H₂O). The required fractions were lyophilized to get Compound Va-1 as white solid 500 mg (27.8%).

LCMS (ESI): m/z calculated for C₄₅H₅₄FN₇O₁₁⁺ [M+H]: 888.39; found: 887.8.

HPLC: Purity: 98.47% (max). ¹HNMR (400 MHz, DMSO-d6): δ 8.53 (t, J=6.7 Hz, 1H), 8.22 (d, J=7.2 Hz, 1H), 8.02 (d, J=10.8 Hz, 1H), δ 7.97 (d, J=8.00 Hz, 1H), 7.86 (t, J=2.80 Hz, 2H), 7.31 (s, 1H), 7.00 (s, 1H), 6.53 (s, 1H), 5.44 (s, 2H), 5.28 (s, 2H), 4.58-4.50 (m, 2H), 4.19 (t, J=7.20 Hz, 3H), 3.42 (t, J=23.20 Hz, 2H), 3.30 (t, J=53.60 Hz, 2H), 3.20-3.18 (m, 2H), 2.07 (t, J=7.20 Hz, 2H), 1.87 (t, J=8.00 Hz, 2H), 1.69 (m, 4H), 1.49-1.42 (m, 11H), 0.90 (t, J=Hz, 3H),

### Example 4. Alternative Method for Synthesizing Compound IIIa-1

Boron trifluoride etherate (BF₃·OEt₂) was found to yield better result than TFA when used as the acid in Step 4 of Example 3 and give higher purity of crude product which makes the purification of Compound IIIa-1 more feasible.

To a stirred solution of Compound II (2.5 g, 5.525 mml, 1.0 eq.) and Compound Aa-1 (Pharmaron Beijing Co., LTD) (3.77 g, 1.3 eq.) in DMSO (63 ml) was added boron trifluoride etherate (BF₃·OEt₂) (1.57 g, 2 eq.). The reaction mixture allowed to stir for 14-18 h at room temperature and added Methyl tert-butyl ether (MTBE, 25 mL) and water (200 mL). The reaction mixture was stirred for 1-2 h and filtered. The filter cake was washed with water (20 mL), dried under vacuum for 1-5 hr and purified by silica gel column to give Compound IIIa-1 (3.1 g).

### Example 5. Synthesis of Compound Vb-1

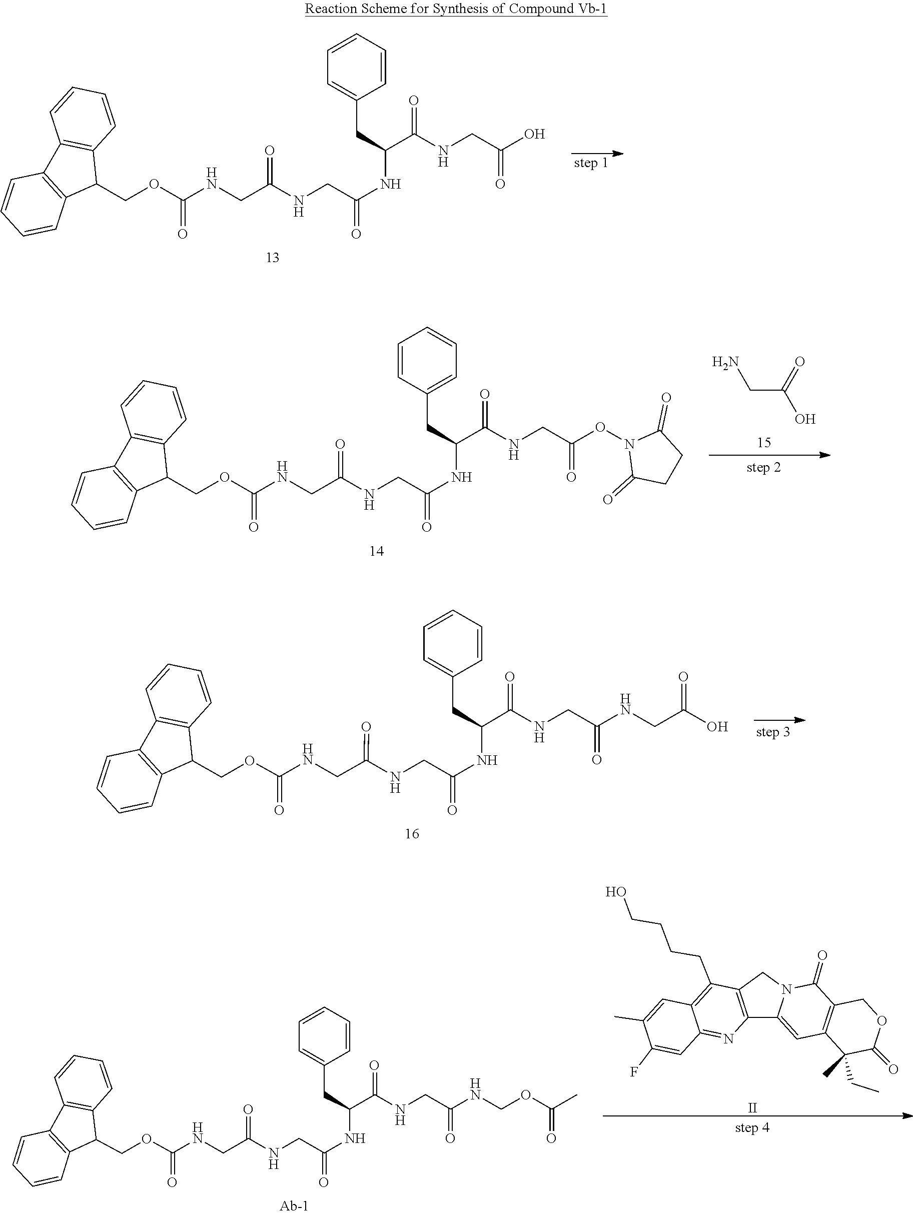

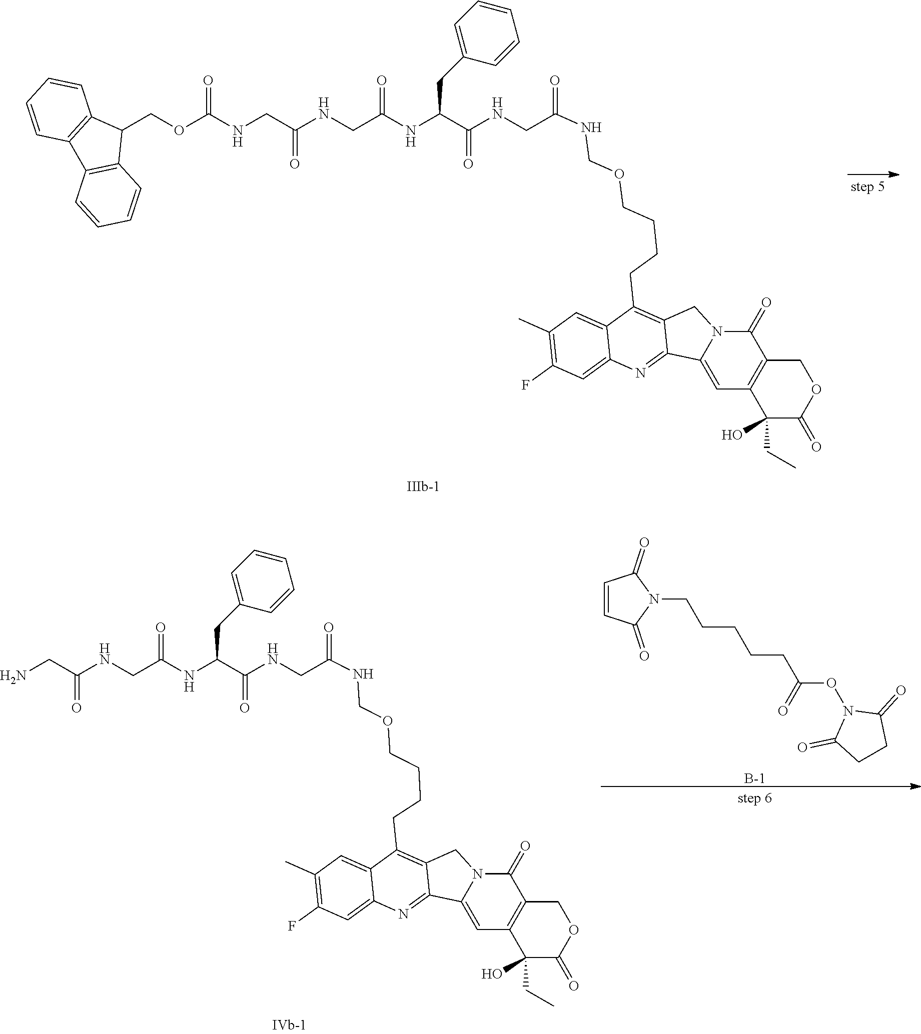

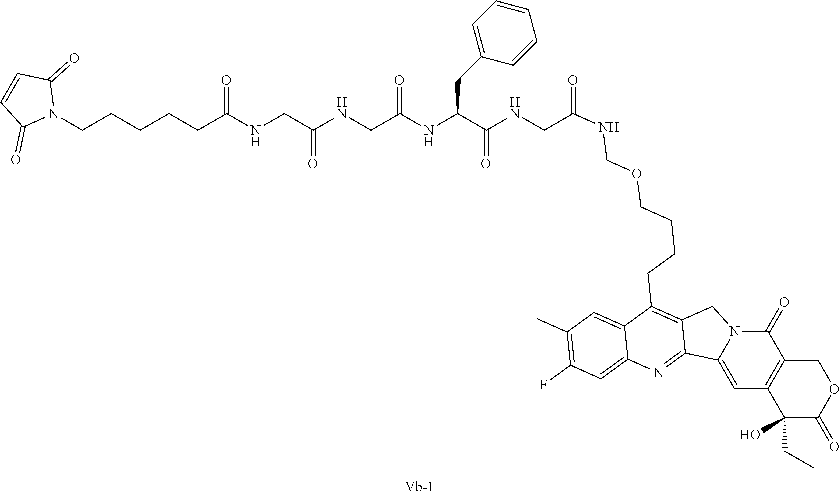 [SMILES: CC[C@@]1(O)C(=O)OCc2c1cc1n(c2=O)Cc2c-1nc1cc(F)c(C)cc1c2CCCCOCNC(=O)CNC(=O)[C@H](Cc1ccccc1)NC(=O)CNC(=O)CNC(=O)CCCCCN1C(=O)C=CC1=O]

### Step 1: Synthesis of Compound 14

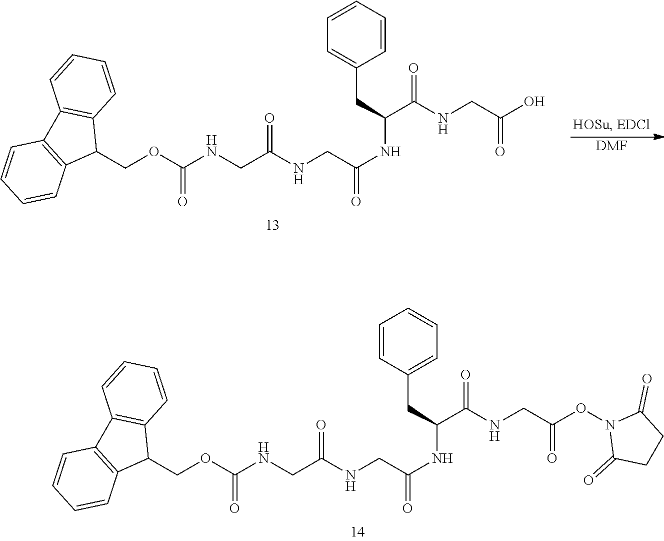

A solution of Compound 13 (10.52 g) in DMF (100 ml) was treated with NHS (2.6 g). After stirring at room temperature for 10 minutes, EDCl (7.2 g) was added to the reaction mixture. The resultant solution continued to stir at room temperature for 4-5 hours. After the consumption of starting material, the reaction mixture was slowly added to MTBE. White solid was precipitated out, which was filtered and dried under reduced pressure. No further purification was required, and the crude was used directly for the next step. 9.42 g crude product (Compound 14) was obtained.

### Step 2: Synthesis of Compound 16

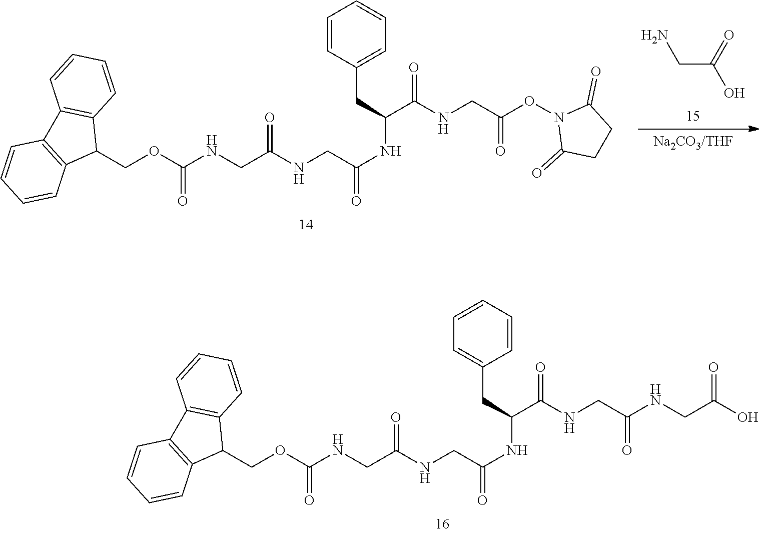 [SMILES: O=C(CNC(=O)OCC1c2ccccc2-c2ccccc21)NCC(=O)N[C@@H](Cc1ccccc1)C(=O)NCC(=O)ON1C(=O)CCC1=O ; NCC(=O)O ; O=C(O)CNC(=O)CNC(=O)[C@H](Cc1ccccc1)NC(=O)CNC(=O)CNC(=O)OCC1c2ccccc2-c2ccccc21]

A solution of Compound 15 (3.16 g) in THF (27.5 ml) and H₂O (68.5 ml) was treated with Na₂CO₃ (2.67 g) portion wise. The reaction temperature was adjusted to 0° C. Compound 14 obtained from Step 1 was then added, and the reaction mixture continued to stir for 5-6 hours at room temperature. The resultant solution was acidified to pH 3. Solid was precipitated out and filtered. The solid was dried overnight to give 20 g crude product (Compound 16).

### Steps 3 and 4: Synthesis of Compound IIIb-1

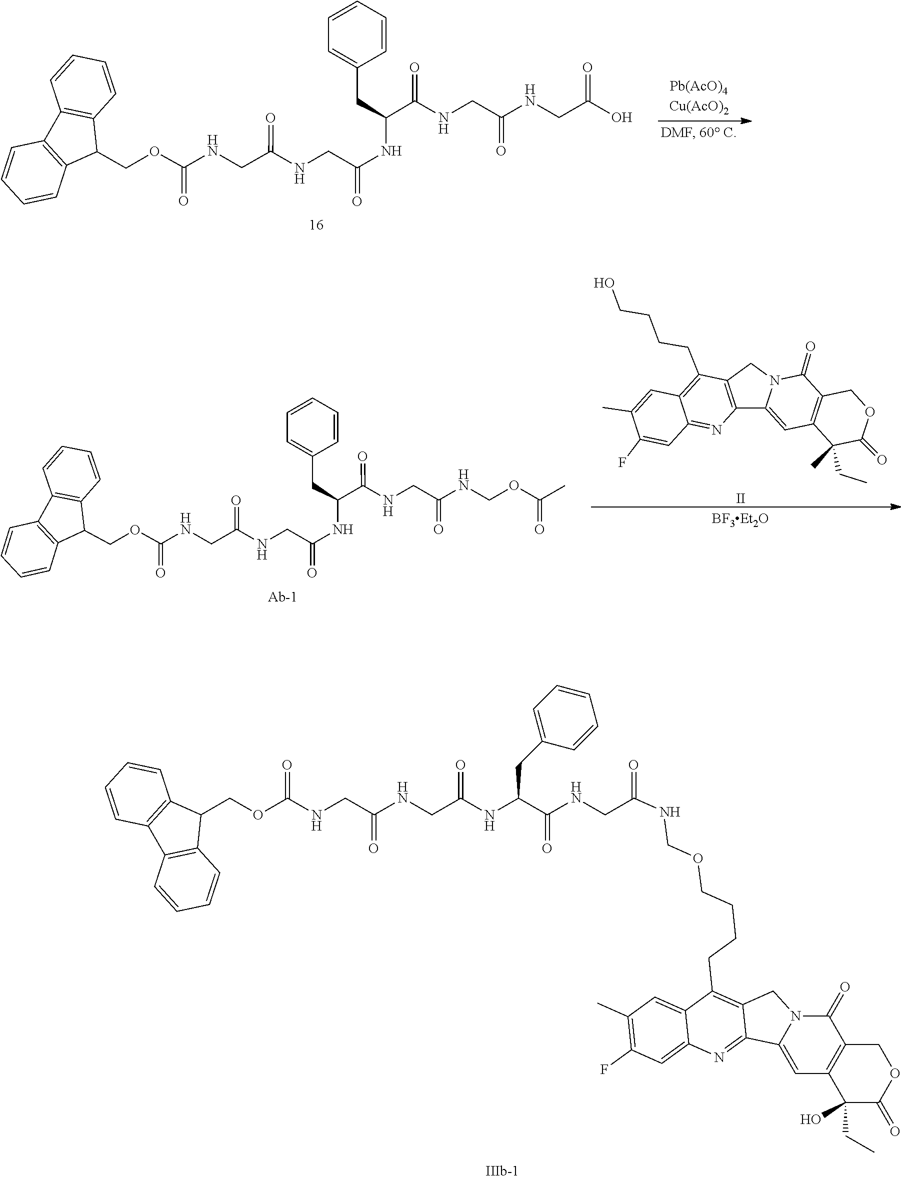 [SMILES: O=C(O)CNC(=O)CNC(=O)[C@H](Cc1ccccc1)NC(=O)CNC(=O)CNC(=O)OCC1c2ccccc2-c2ccccc21 ; CC(=O)OCNC(=O)CNC(=O)[C@H](Cc1ccccc1)NC(=O)CNC(=O)CNC(=O)OCC1c2ccccc2-c2ccccc21 ; CC[C@@]1(C)C(=O)OCc2c1cc1n(c2=O)Cc2c-1nc1cc(F)c(C)cc1c2CCCCO ; "II" ; CC[C@@]1(O)C(=O)OCc2c1cc1n(c2=O)Cc2c-1nc1cc(F)c(C)cc1c2CCCCOCNC(=O)CNC(=O)[C@H](Cc1ccccc1)NC(=O)CNC(=O)CNC(=O)OCC1c2ccccc2-c2ccccc21]

A solution of Compound 16 (300 mg) in DMF (5.5 mL) was treated with Pb(OAc)₄ (374 mg), Cu(OAc)₂, and AcOH (0.07 mL) at room temperature and then the reaction temperature was raised to 60° C. After stirring for 15 minutes, the reaction mixture was quenched with bicarbonate solution. The resultant solution was extracted with EA. The combined organic layers were evaporated to provide crude product (Compound Ab-1) which was directly used for the next step.

Compound II was dissolved in DMF (5 mL) and the resultant solution stirred for 10 minutes. BF₃·Et₂O was added to the solution followed by Compound Ab-1. The reaction mixture continued to stir for 20 hours at room temperature and was extracted with EA. The combined organic layers were evaporated and the crude was purified by column chromatography to provide 180 mg pure product (Compound IIIb-1).

### Steps 5 and 6: Synthesis of Compound Vb-1

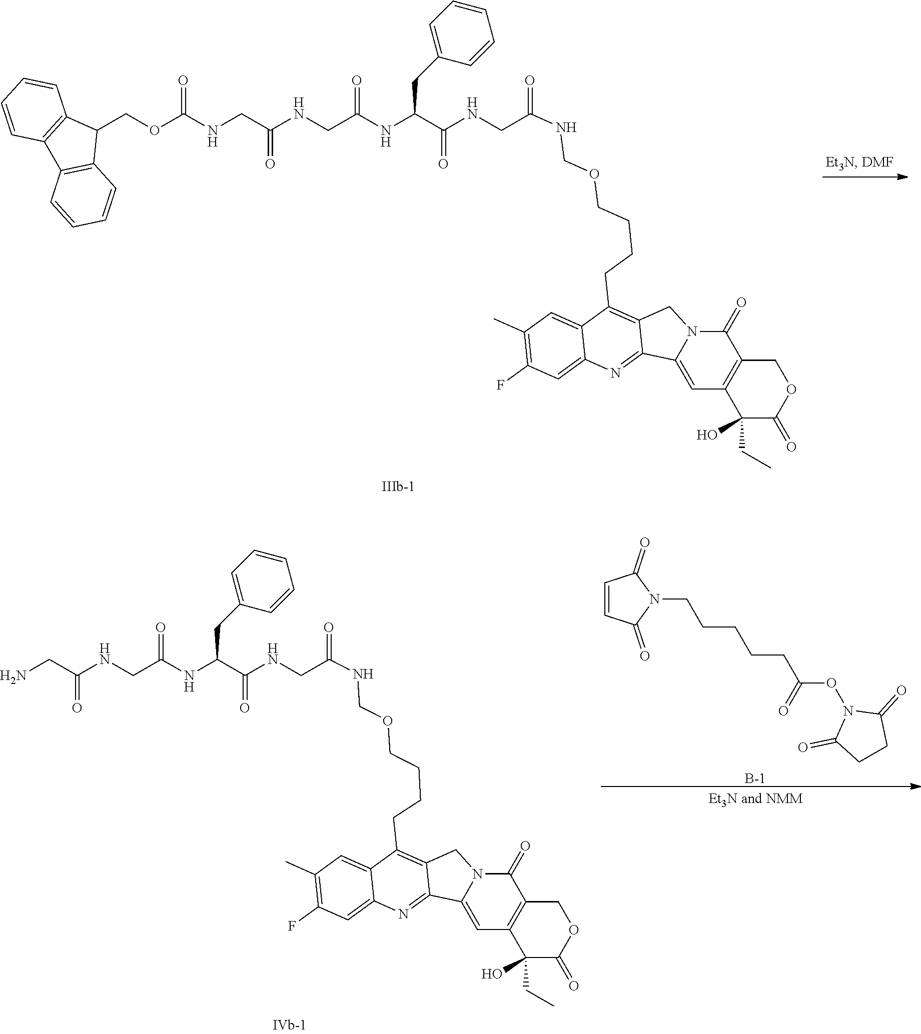

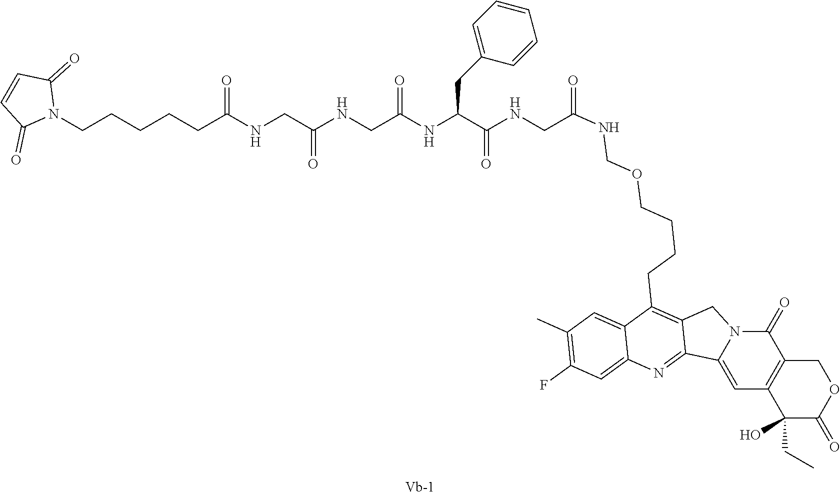 [SMILES: CC[C@@]1(O)C(=O)OCc2c1cc1n(c2=O)Cc2c-1nc1cc(F)c(C)cc1c2CCCCOCNC(=O)CNC(=O)[C@H](Cc1ccccc1)NC(=O)CNC(=O)CNC(=O)CCCCCN1C(=O)C=CC1=O]

A solution of Compound IIIb-1 (1 g) in DMF (5.3 mL) was treated with Et₃N (2.86 mL) at room temperature. The resultant mixture stirred for 24 hours and was then concentrated to give crude product (Compound IVb-1) which was directly used for the next step.

The crude product (Compound IVb-1) was dissolved in DMF (7 mL) and then added Compound B-1 (312 mg) and NMM (0.2 mL). The resultant mixture continued to stir. Reaction was complete in 2 hours. The reaction mixture was purified by preparative HPLC to provide 120 mg product (Compound Vb-1).

1. A method of preparing a compound of Formula (II): 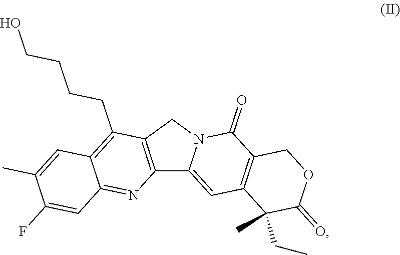 [SMILES: CC[C@@]1(C)C(=O)OCc2c1cc1n(c2=O)Cc2c-1nc1cc(F)c(C)cc1c2CCCCO]
comprising the step of reacting a compound of Formula (I): 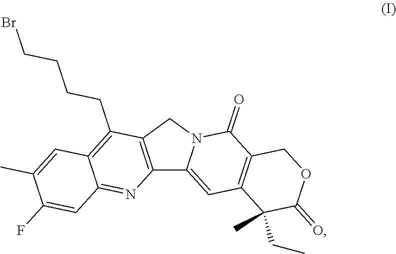 [SMILES: CC[C@@]1(C)C(=O)OCc2c1cc1n(c2=O)Cc2c-1nc1cc(F)c(C)cc1c2CCCCBr]
with water and N-methyl-2-pyrollidone (NMP) to form the compound of Formula (II).

2. The method of claim 1, wherein the reaction is carried out at a temperature between 20° C. and 150° C.

3. The method of claim 2, wherein the reaction is carried out at a temperature between 30° C. and 150° C., between 70° C. and 120° C., or between 80° C. and 120° C.

4. The method of claim 3, wherein the reaction is carried out at 100° C.

5. The method of any one of claims 1 to 4, wherein the compound of Formula (I) is prepared by reacting a compound of Formula (C): 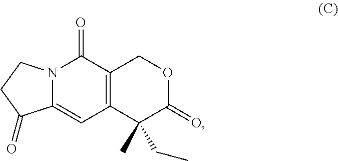 [SMILES: CC[C@@]1(C)C(=O)OCc2c1cc1n(c2=O)CCC1=O]
with a compound of Formula (D): 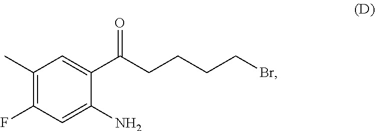 [SMILES: Cc1cc(C(=O)CCCCBr)c(N)cc1F]
to form the compound of Formula (I).

6. The method of claim 5, wherein the compound of Formula (D) is prepared by reacting a compound of Formula (E) and a compound of Formula (F): 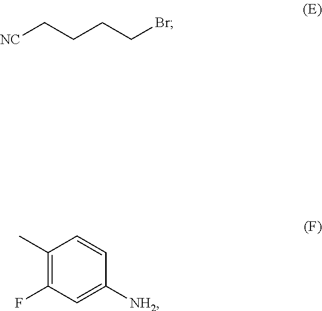
to form the compound of Formula (D).

7. The method of claim 6, wherein the reaction between the compound of Formula (E) and the compound of Formula (F) is carried out in the presence of boron trichloride (BCl₃) and aluminum trichloride (AlCl₃).

8. A method of preparing a compound of Formula (III): 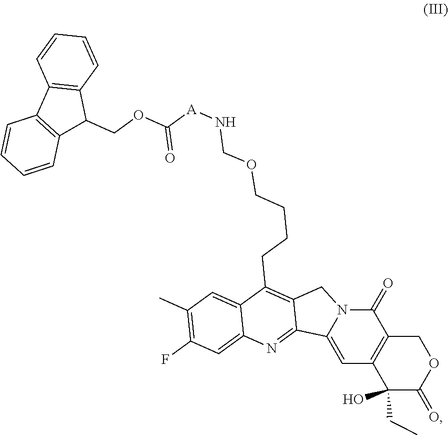
comprising reacting a compound of Formula (II): 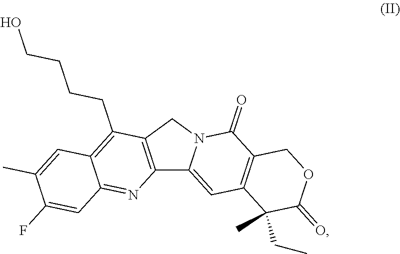 [SMILES: CC[C@@]1(C)C(=O)OCc2c1cc1n(c2=O)Cc2c-1nc1cc(F)c(C)cc1c2CCCCO]
with a compound of Formula (A): 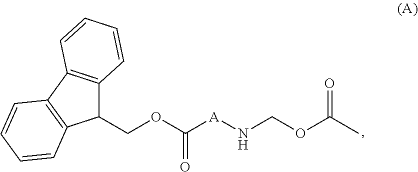
in the presence of trifluoroacetic acid (TFA) to form the compound of Formula (III), wherein A is a peptide comprising 2 to 10 amino acids.

9. A method of preparing a compound of Formula (II): 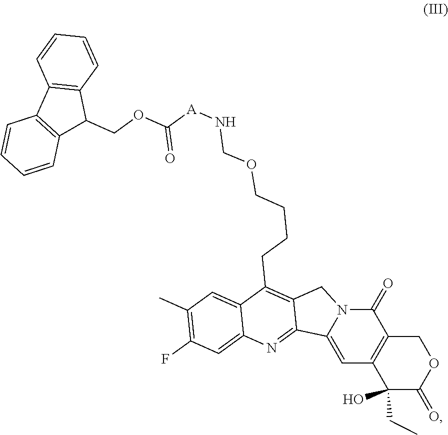
comprising reacting a compound of Formula (II): 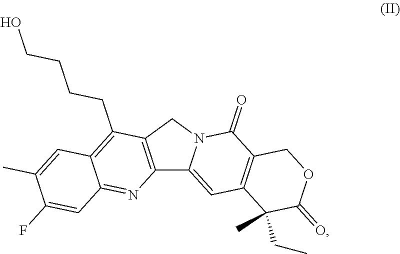 [SMILES: CC[C@@]1(C)C(=O)OCc2c1cc1n(c2=O)Cc2c-1nc1cc(F)c(C)cc1c2CCCCO]
with a compound of Formula (A): 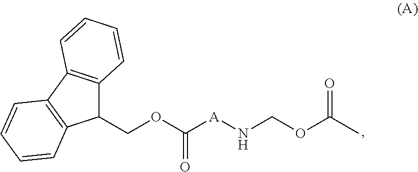
in the presence of a Lewis acid to form the compound of Formula (III), wherein A is a peptide comprising 2 to 10 amino acids.

10. The method of claim 9, wherein the Lewis acid is boron trifluoride etherate (BF₃·OEt₂), boron trichloride (BCl₃), or aluminum trichloride (AlCl₃).

11. The method of claim 9, wherein the Lewis acid is boron trifluoride etherate (BF₃·OEt₂).

12. The method of any one of claims 8-11, wherein A is a peptide comprising 2 to 4 amino acids.

13. The method of claim 12, wherein A is:
-Ala-Ala-*,
-Ala-Val-*,
-Val-Ala-*,
-Val-Cit-*,
-Cit-Val-*,
-Gln-Leu-*,
-Leu-Gln-*,
-Ala-Ala-Ala-*,
-Ala-Ala-Ala-Ala-*,
-Gly-Ala-Gly-Gly-*,
-Gly-Gly-Ala-Gly-*,
-Gly-Val-Gly-Gly-*,
-Gly-Gly-Val-Gly-*,
-Gly-Phe-Gly-Gly-*, or
-Gly-Gly-Phe-Gly-*, wherein * is the point of attachment to the carbonyl (—C(═O)—) group in Formula (A).

14. The method of claim 13, wherein A is -Val-Cit-*, -Cit-Val-*, -Ala-Ala-Ala-*, -Gly-Phe-Gly-Gly-* or -Gly-Gly-Phe-Gly-*.

15. The method of any one of claims 8-12, wherein the compound of Formula (III) is represented by Formula (IIIa): 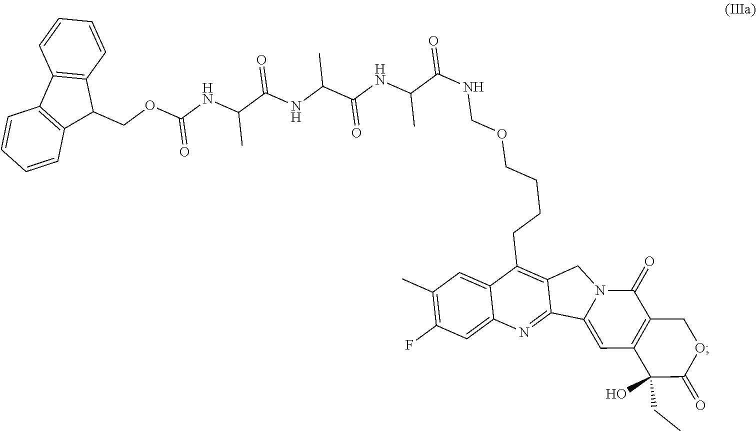 [SMILES: CC[C@@]1(O)C(=O)OCc2c1cc1n(c2=O)Cc2c-1nc1cc(F)c(C)cc1c2CCCCOCNC(=O)C(C)NC(=O)C(C)NC(=O)C(C)NC(=O)OCC1c2ccccc2-c2ccccc21]
and
the compound of Formula (A) is represented by Formula (Aa):

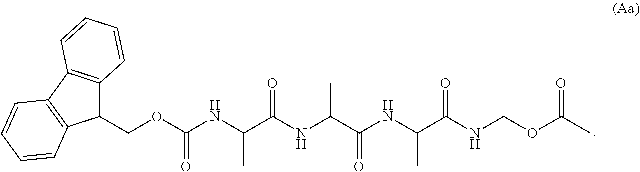 [SMILES: CC(=O)OCNC(=O)C(C)NC(=O)C(C)NC(=O)C(C)NC(=O)OCC1c2ccccc2-c2ccccc21]

16. The method of claim 15, wherein the compound of Formula (IIIa) is represented by Formula (IIIa-1): 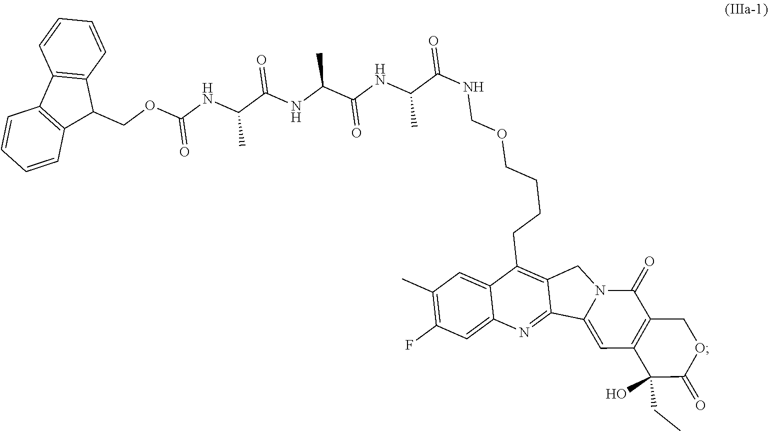 [SMILES: CC[C@@]1(O)C(=O)OCc2c1cc1n(c2=O)Cc2c-1nc1cc(F)c(C)cc1c2CCCCOCNC(=O)[C@H](C)NC(=O)[C@H](C)NC(=O)[C@H](C)NC(=O)OCC1c2ccccc2-c2ccccc21]
and
the compound of Formula (Aa) is represented by Formula (Aa-1):

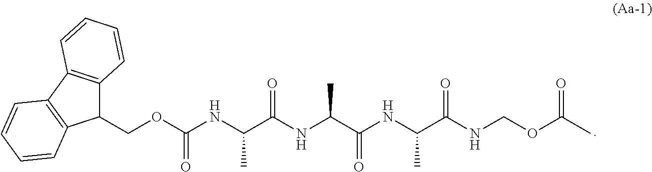 [SMILES: CC(=O)OCNC(=O)[C@H](C)NC(=O)[C@H](C)NC(=O)[C@H](C)NC(=O)OCC1c2ccccc2-c2ccccc21]

17. The method of claim 15, wherein the compound of Formula (IIIa) is represented by Formula (IIIa-2): 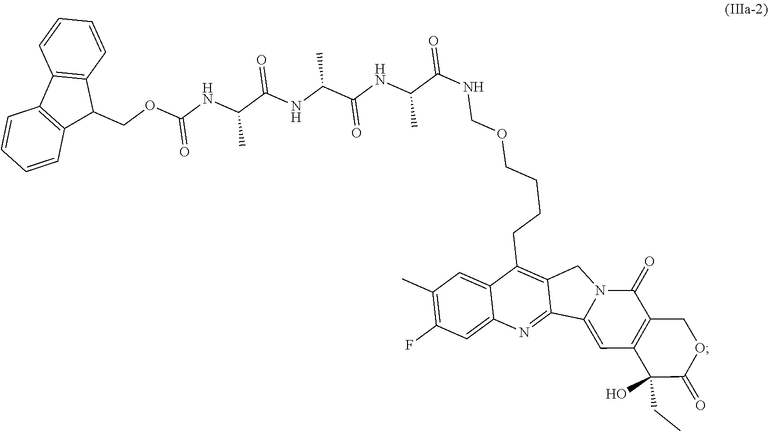 [SMILES: CC[C@@]1(O)C(=O)OCc2c1cc1n(c2=O)Cc2c-1nc1cc(F)c(C)cc1c2CCCCOCNC(=O)[C@H](C)NC(=O)[C@@H](C)NC(=O)[C@H](C)NC(=O)OCC1c2ccccc2-c2ccccc21]
and
the compound of Formula (Aa) is represented by Formula (Aa-2):

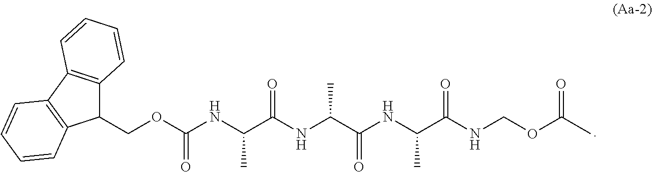 [SMILES: CC(=O)OCNC(=O)[C@H](C)NC(=O)[C@@H](C)NC(=O)[C@H](C)NC(=O)OCC1c2ccccc2-c2ccccc21]

18. The method of any one of claims 8-12, wherein the compound of Formula (III) is represented by Formula (IIIb) or (IIIc): 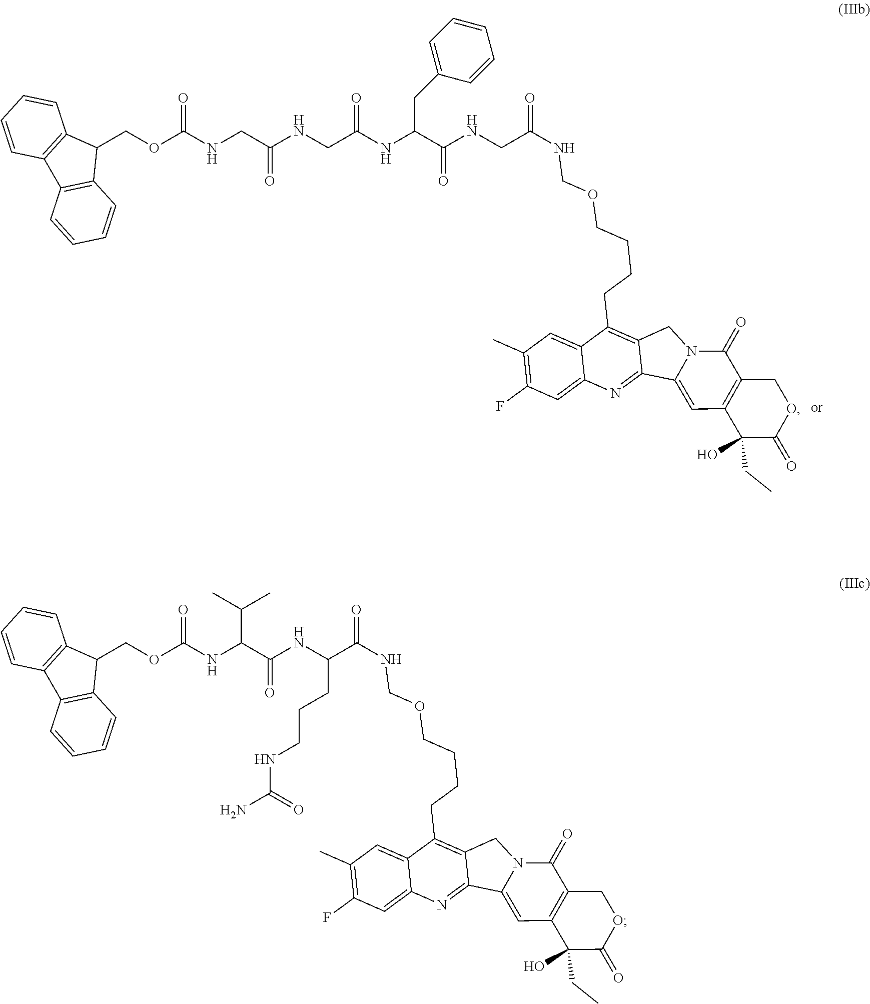 [SMILES: CC[C@@]1(O)C(=O)OCc2c1cc1n(c2=O)Cc2c-1nc1cc(F)c(C)cc1c2CCCCOCNC(=O)CNC(=O)C(Cc1ccccc1)NC(=O)CNC(=O)CNC(=O)OCC1c2ccccc2-c2ccccc21 ; "or" ; CC[C@@]1(O)C(=O)OCc2c1cc1n(c2=O)Cc2c-1nc1cc(F)c(C)cc1c2CCCCOCNC(=O)C(CCCNC(N)=O)NC(=O)C(NC(=O)OCC1c2ccccc2-c2ccccc21)C(C)C]
and
the compound of Formula (A) is represented by Formula (Ab) or (Ac):

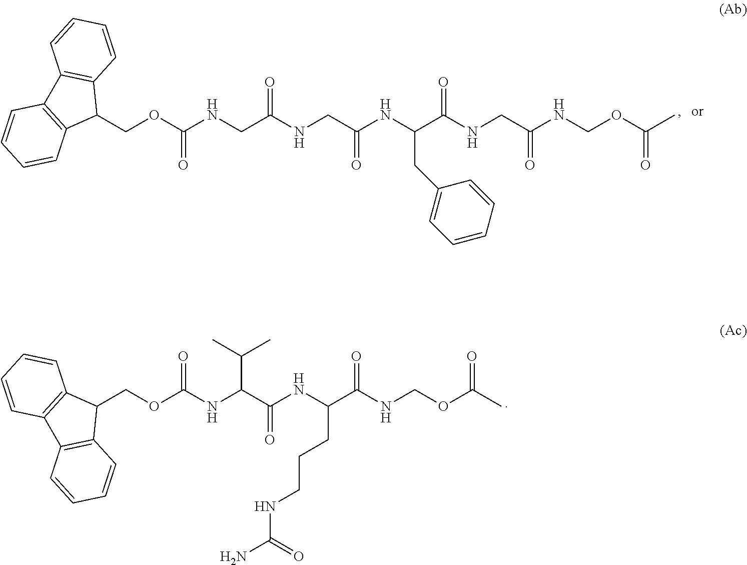 [SMILES: CC(=O)OCNC(=O)CNC(=O)C(Cc1ccccc1)NC(=O)CNC(=O)CNC(=O)OCC1c2ccccc2-c2ccccc21 ; CC(=O)OCNC(=O)C(CCCNC(N)=O)NC(=O)C(NC(=O)OCC1c2ccccc2-c2ccccc21)C(C)C]

19. The method of claim 18, wherein the compound of Formula (IIIb) is represented by Formula (IIIb-1) and the compound of Formula (IIIc) is represented by Formula (IIIc-1): 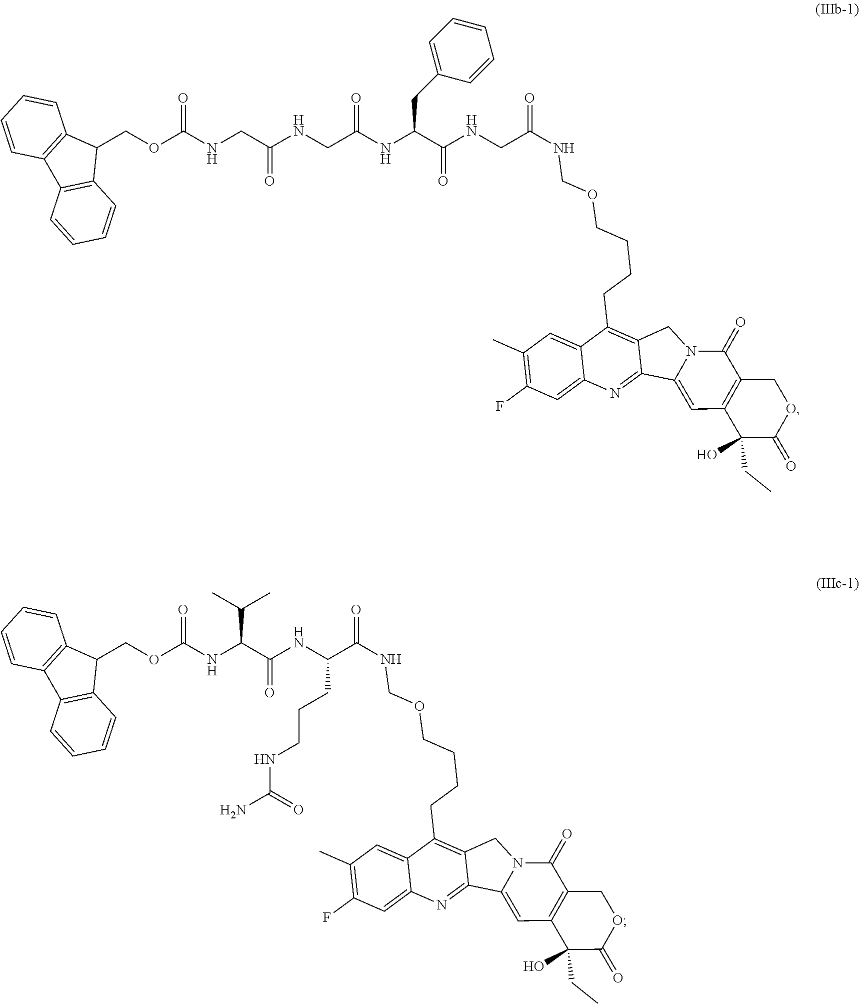 [SMILES: CC[C@@]1(O)C(=O)OCc2c1cc1n(c2=O)Cc2c-1nc1cc(F)c(C)cc1c2CCCCOCNC(=O)CNC(=O)[C@H](Cc1ccccc1)NC(=O)CNC(=O)CNC(=O)OCC1c2ccccc2-c2ccccc21 ; CC[C@@]1(O)C(=O)OCc2c1cc1n(c2=O)Cc2c-1nc1cc(F)c(C)cc1c2CCCCOCNC(=O)[C@H](CCCNC(N)=O)NC(=O)[C@@H](NC(=O)OCC1c2ccccc2-c2ccccc21)C(C)C]
and
the compound of Formula (Ab) is represented by Formula (Ab-1) and the compound of Formula (Ac) is represented by Formula (Ac-1):

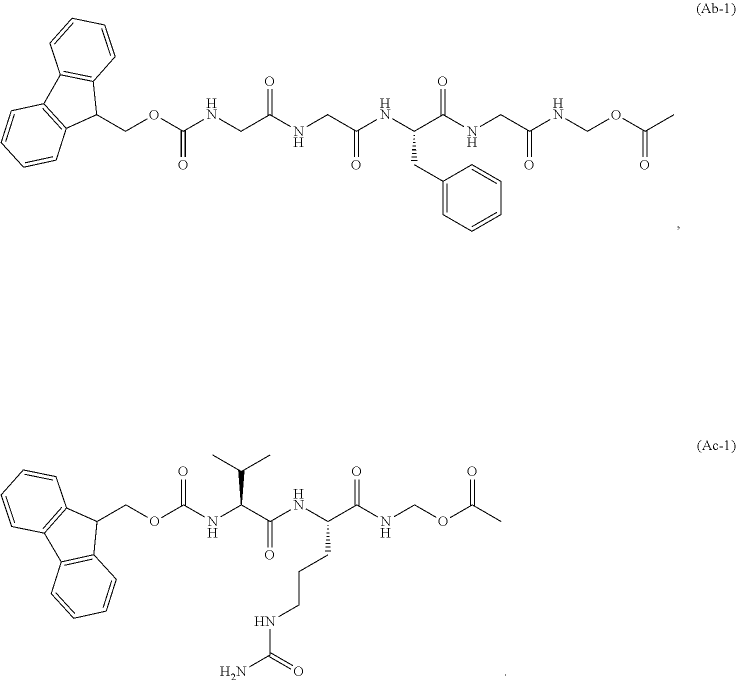 [SMILES: CC(=O)OCNC(=O)CNC(=O)[C@H](Cc1ccccc1)NC(=O)CNC(=O)CNC(=O)OCC1c2ccccc2-c2ccccc21 ; CC(=O)OCNC(=O)[C@H](CCCNC(N)=O)NC(=O)[C@@H](NC(=O)OCC1c2ccccc2-c2ccccc21)C(C)C]

20. The method of any one of claims 8 to 19, wherein the compound of Formula (III) is further reacted with a base to form the compound of Formula (IV):

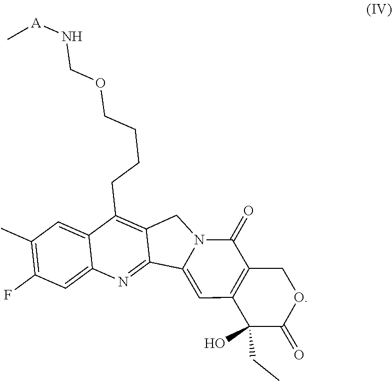

21. The method of claim 20, wherein the compound of Formula (III) is represented by Formula (IIIa), (IIIa-1), (IIIa-2), (IIIb), (IIIb-1), (IIIc) or (IIIc-1) and the compound of Formula (IV) is represented by Formula (IVa), (IVa-1), (IVa-2), (IVb), (IVb-1), (IVc) or (IVc-1) respectively:

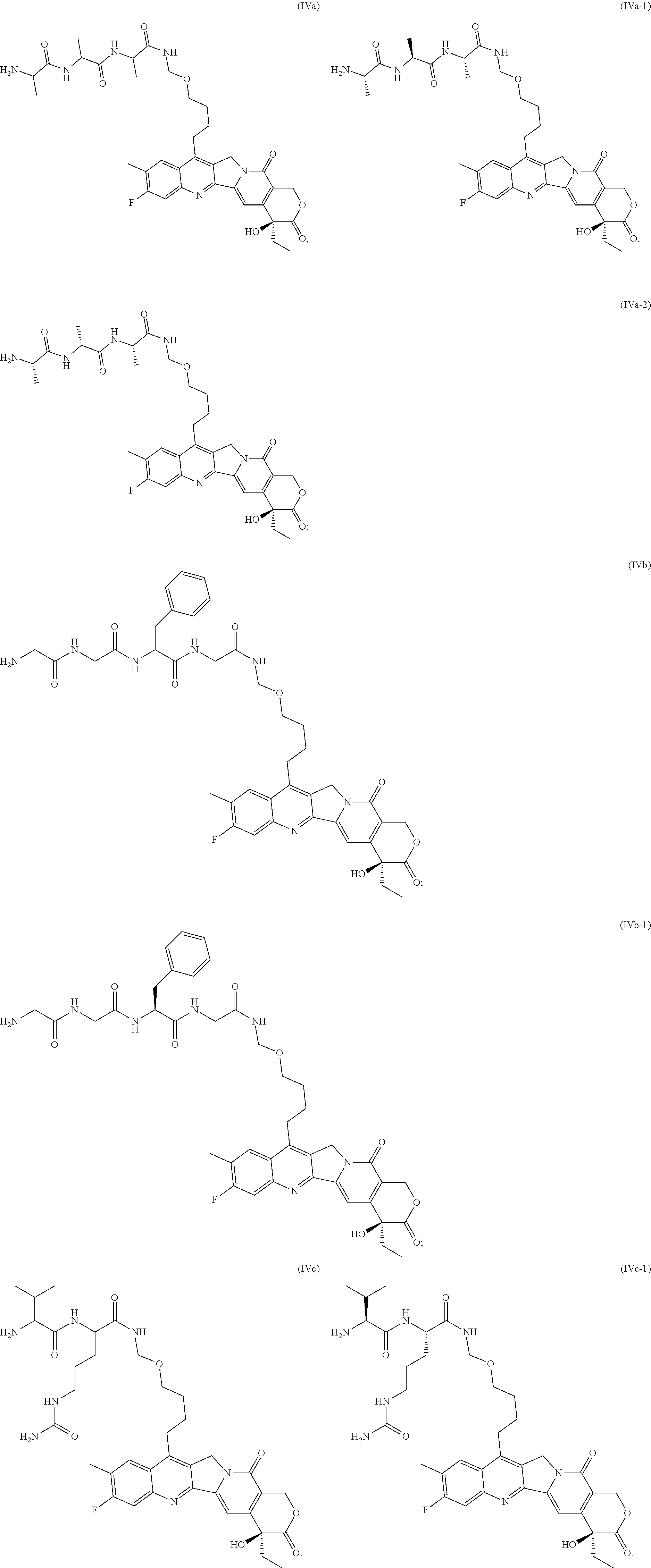

22. The method of claim 20 or 21, wherein the base is selected from piperidine, morpholine, N-methylmorpholine, 4-methylpiperidine, piperazine, pyrrolidine, 1,8-diazabicyclo[5.4.0]undec-7-ene (DBU), diisopropylethyalamine (DIPEA), triethylamine (TEA), diethylamine (DEA), and a combination thereof.

23. The method of claim 22, wherein the base is triethylamine (TEA), piperidine, morpholine, or a combination thereof.

24. The method of any one of claims 20 to 23, wherein the compound of Formula (IV) is reacted with a compound of Formula (B): 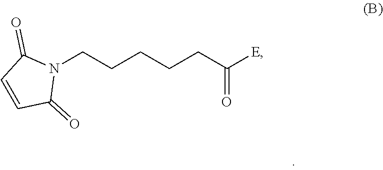
to form a compound of Formula (V): 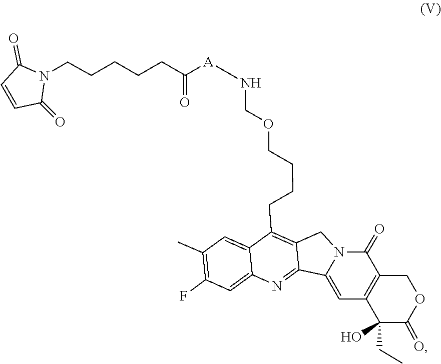
wherein E is —OH, —Cl or

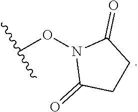 [SMILES: *ON1C(=O)CCC1=O]

25. The method of claim 24, wherein E is 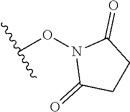 [SMILES: *ON1C(=O)CCC1=O]
and the reaction between the compound of Formula (IV) and the compound of Formula (B) is carried out in the presence of a base.

26. The method of claim 25, wherein the base is N-methylmorpholine, triethylamine (TEA), 1,8-diazabicyclo[5.4.0]undec-7-ene (DBU), N-methylpiperidine, 1-methylpyrrolidine, or diisopropylethyalamine (DIPEA).

27. The method of claim 24, wherein E is —OH and the reaction between the compound of Formula (IV) and the compound of Formula (B) is carried out in the presence of an activating agent.

28. The method of claim 27, wherein the activating agent is selected from 2,4,6-trialkyl-1,3,5,2,4,6-trioxatriphosphorinane 2,4,6-trioxide, carbodiimide (e.g., N,N′-dicyclohexylcarbodiimide (DCC) or 1-ethyl-3-(3-dimethylaminopropyl) carbodiimide (EDC)), 1,1′-carbonyldiimidazole (CDI), a uronium, an activated ester, a phosphonium, 2-alkyl-1-alkylcarbonyl-1,2-dihydroquinoline, 2-alkoxy-1-alkoxycarbonyl-1,2-dihydroquinoline, or alkylchloroformate.

29. The method of claim 28, wherein the activating agent is 2,4,6-tripropyl-1,3,5,2,4,6-trioxatriphosphorinane 2,4,6-trioxide (T3P).

30. The method of claim 27, wherein the reaction is carried out in the presence of 1-ethyl-3-(3-dimethylaminopropyl) carbodiimide (EDC) and hydroxybenzotriazole (HOBt).

31. The method of any one of claims 24 to 30, wherein the compound of Formula (IV) is represented by Formula (IVa), (IVa-1), (IVa-2), (IVb), (IVb-1), (IVc) or (IVc-1) and the compound of Formula (V) is represented by Formula (Va), (Va-1), (Va-2), (Vb), (Vb-1), (Vc), or (Vc-1) respectively:

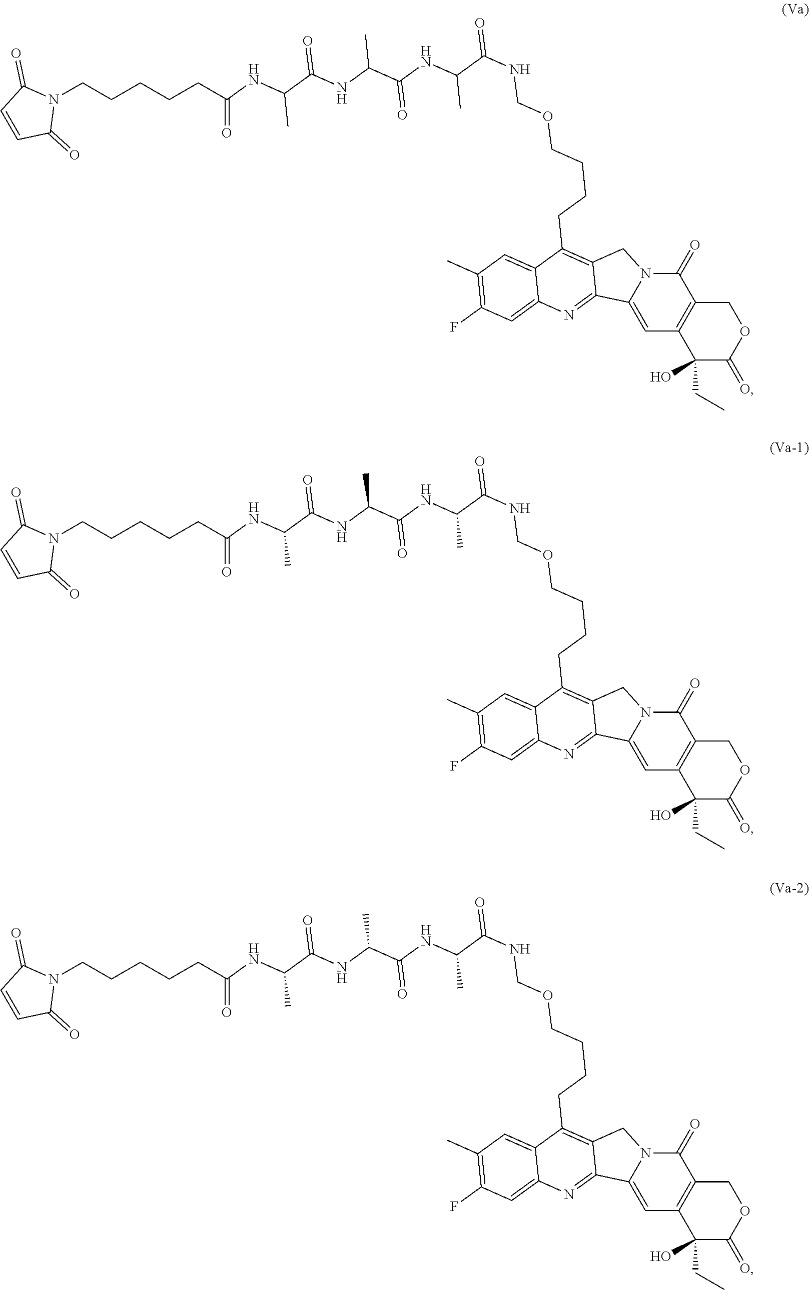 [SMILES: CC[C@@]1(O)C(=O)OCc2c1cc1n(c2=O)Cc2c-1nc1cc(F)c(C)cc1c2CCCCOCNC(=O)C(C)NC(=O)C(C)NC(=O)C(C)NC(=O)CCCCCN1C(=O)C=CC1=O ; CC[C@@]1(O)C(=O)OCc2c1cc1n(c2=O)Cc2c-1nc1cc(F)c(C)cc1c2CCCCOCNC(=O)[C@H](C)NC(=O)[C@H](C)NC(=O)[C@H](C)NC(=O)CCCCCN1C(=O)C=CC1=O ; CC[C@@]1(O)C(=O)OCc2c1cc1n(c2=O)Cc2c-1nc1cc(F)c(C)cc1c2CCCCOCNC(=O)[C@H](C)NC(=O)[C@@H](C)NC(=O)[C@H](C)NC(=O)CCCCCN1C(=O)C=CC1=O]

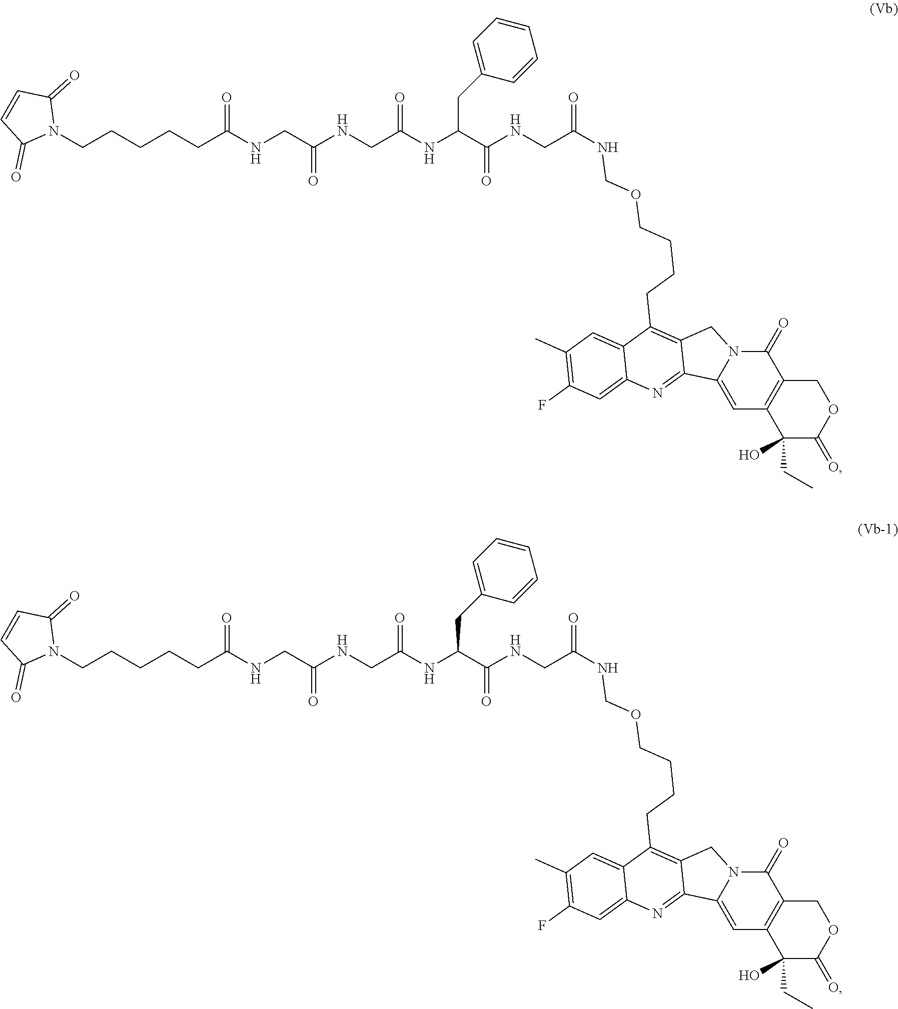 [SMILES: CC[C@@]1(O)C(=O)OCc2c1cc1n(c2=O)Cc2c-1nc1cc(F)c(C)cc1c2CCCCOCNC(=O)CNC(=O)C(Cc1ccccc1)NC(=O)CNC(=O)CNC(=O)CCCCCN1C(=O)C=CC1=O ; CC[C@@]1(O)C(=O)OCc2c1cc1n(c2=O)Cc2c-1nc1cc(F)c(C)cc1c2CCCCOCNC(=O)CNC(=O)[C@H](Cc1ccccc1)NC(=O)CNC(=O)CNC(=O)CCCCCN1C(=O)C=CC1=O]

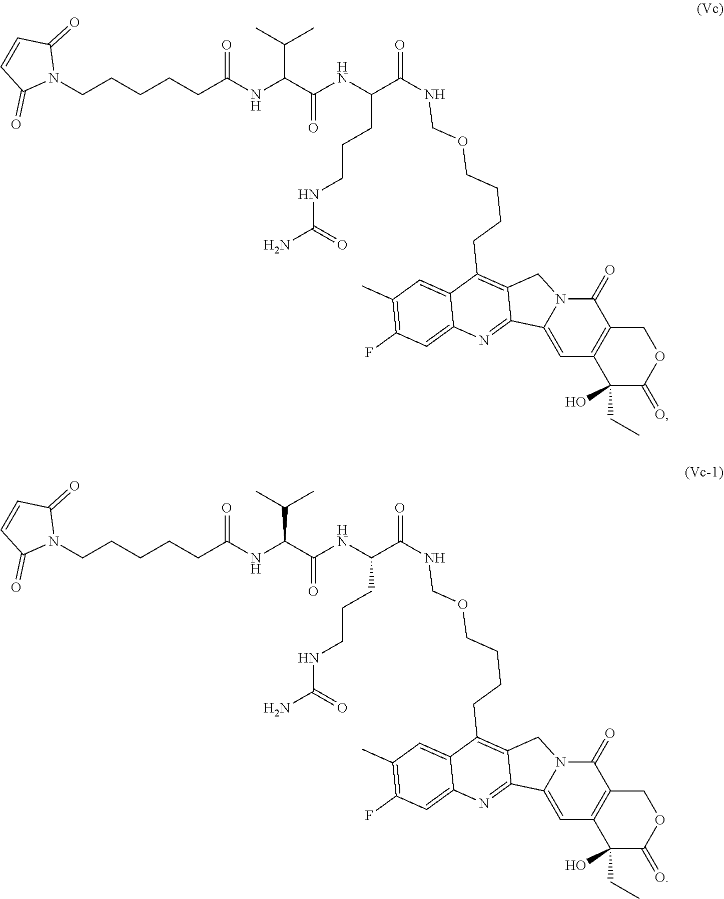 [SMILES: CC[C@@]1(O)C(=O)OCc2c1cc1n(c2=O)Cc2c-1nc1cc(F)c(C)cc1c2CCCCOCNC(=O)C(CCCNC(N)=O)NC(=O)C(NC(=O)CCCCCN1C(=O)C=CC1=O)C(C)C ; CC[C@@]1(O)C(=O)OCc2c1cc1n(c2=O)Cc2c-1nc1cc(F)c(C)cc1c2CCCCOCNC(=O)[C@H](CCCNC(N)=O)NC(=O)[C@@H](NC(=O)CCCCCN1C(=O)C=CC1=O)C(C)C]

32. A method of preparing a compound of Formula (VII): 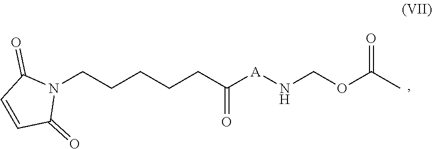
comprising the steps of:
(a) deprotecting a compound of Formula (A): 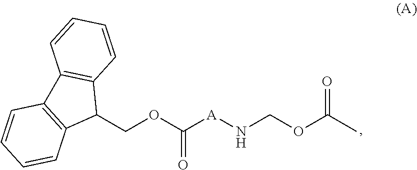
with a base to form a compound of Formula (VI): 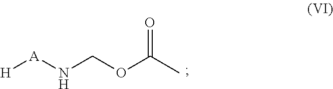
(b) reacting the compound of Formula (VI) with a compound of Formula (B): 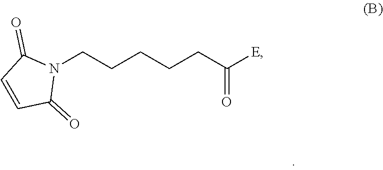
wherein A is a peptide comprising 2 to 10 amino acids; and E is —OH, —Cl or

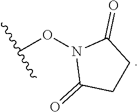 [SMILES: *ON1C(=O)CCC1=O]

33. The method of claim 32, wherein the base in step (a) is selected from piperidine, morpholine, N-methylmorpholine, 4-methylpiperidine, piperazine, pyrrolidine, 1,8-diazabicyclo[5.4.0]undec-7-ene (DBU), diisopropylethyalamine (DIPEA), triethylamine (TEA), diethylamine (DEA), and a combination thereof.

34. The method of claim 33, wherein the base is triethylamine (TEA), piperidine, morpholine, or a combination thereof.

35. The method of any one of claims 32-34, wherein E is 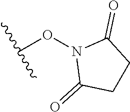 [SMILES: *ON1C(=O)CCC1=O]
and the reaction between the compound of Formula (VI) and the compound of Formula (B) in step (b) is carried out in the presence of a base.

36. The method of claim 35, wherein the base in step (b) is N-methylmorpholine, triethylamine (TEA), 1,8-diazabicyclo[5.4.0]undec-7-ene (DBU), N-methylpiperidine, 1-methylpyrrolidine, or diisopropylethyalamine (DIPEA).

37. The method of any one of claims 32-34, wherein E is —OH and the reaction between the compound of Formula (VI) and the compound of Formula (B) in step (b) is carried out in the presence of an activating agent.

38. The method of claim 37, wherein the activating agent is selected from 2,4,6-trialkyl-1,3,5,2,4,6-trioxatriphosphorinane 2,4,6-trioxide, carbodiimide (e.g., N,N′-dicyclohexylcarbodiimide (DCC) or 1-ethyl-3-(3-dimethylaminopropyl) carbodiimide (EDC)), 1,1′-carbonyldiimidazole (CDI), a uronium, an activated ester, a phosphonium, 2-alkyl-1-alkylcarbonyl-1,2-dihydroquinoline, 2-alkoxy-1-alkoxycarbonyl-1,2-dihydroquinoline, or alkylchloroformate.

39. The method of claim 38, wherein the activating agent is 2,4,6-tripropyl-1,3,5,2,4,6-trioxatriphosphorinane 2,4,6-trioxide (T3P).

40. The method of claim 37, wherein the reaction between the compound of Formula (VI) and the compound of Formula (B) in step (b) is carried out in the presence of 1-ethyl-3-(3-dimethylaminopropyl)carbodiimide (EDC) and hydroxybenzotriazole (HOBt).

41. The method of any one of claims 32 to 40, wherein A is a peptide comprising 2 to 4 amino acids.

42. The method of claim 41, wherein A is:
-Ala-Ala-*,
-Ala-Val-*,
-Val-Ala-*,
Val-Cit-*,
-Cit-Val-*,
-Gln-Leu-*,
-Leu-Gln-*
-Ala-Ala-Ala-*,
-Ala-Ala-Ala-Ala-*,
-Gly-Ala-Gly-Gly-*,
-Gly-Gly-Ala-Gly-*,
-Gly-Val-Gly-Gly-*,
-Gly-Gly-Val-Gly-*,
-Gly-Phe-Gly-Gly-*, or
-Gly-Gly-Phe-Gly-*, wherein * is the point of attachment to the carbonyl (—C(═O)—) group in Formula (A).

43. The method of claim 42, wherein A is -Val-Cit-*, -Cit-Val-*, -Ala-Ala-Ala-*, -Gly-Phe-Gly-Gly-*, or -Gly-Gly-Phe-Gly-*.

44. The method of any one of claims 32 to 41, wherein the compound of Formula (A) is represented by Formula (Aa); the compound of Formula (VI) is represented by Formula (VIa) and the compound of Formula (VII) is represented by Formula (VIIa):

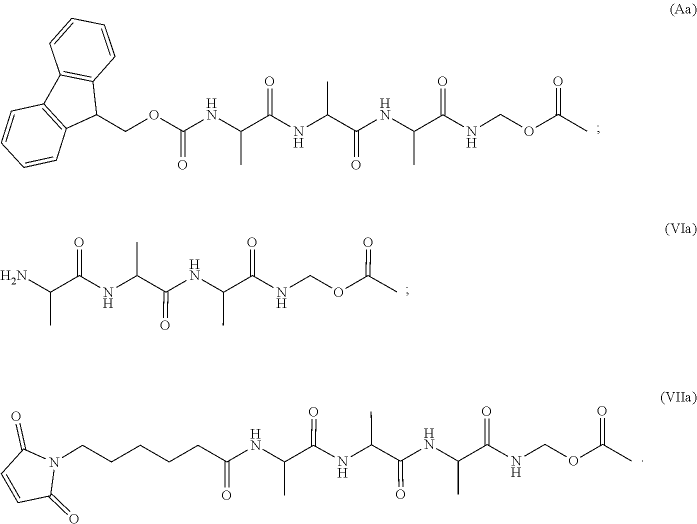 [SMILES: CC(=O)OCNC(=O)C(C)NC(=O)C(C)NC(=O)C(C)NC(=O)OCC1c2ccccc2-c2ccccc21 ; CC(=O)OCNC(=O)C(C)NC(=O)C(C)NC(=O)C(C)N ; CC(=O)OCNC(=O)C(C)NC(=O)C(C)NC(=O)C(C)NC(=O)CCCCCN1C(=O)C=CC1=O]

45. The method of claim 44, wherein the compound of Formula (Aa) is represented by Formula (Aa-1); the compound of Formula (VIa) is represented by Formula (VIa-1) and the compound of Formula (VIIa) is represented by Formula (VIIa-1):

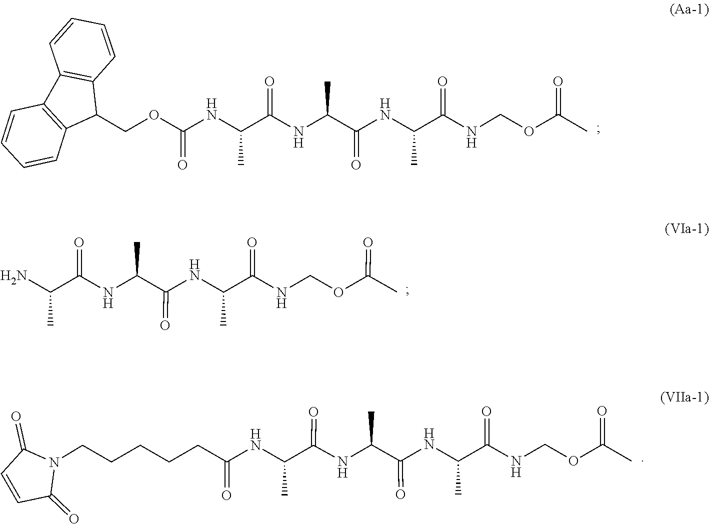 [SMILES: CC(=O)OCNC(=O)[C@H](C)NC(=O)[C@H](C)NC(=O)[C@H](C)NC(=O)OCC1c2ccccc2-c2ccccc21 ; CC(=O)OCNC(=O)[C@H](C)NC(=O)[C@H](C)NC(=O)[C@H](C)N ; CC(=O)OCNC(=O)[C@H](C)NC(=O)[C@H](C)NC(=O)[C@H](C)NC(=O)CCCCCN1C(=O)C=CC1=O]

46. The method of claim 44, wherein the compound of Formula (Aa) is represented by Formula (Aa-2); the compound of Formula (VIa) is represented by Formula (VIa-2) and the compound of Formula (VIIa) is represented by Formula (VIIa-2):

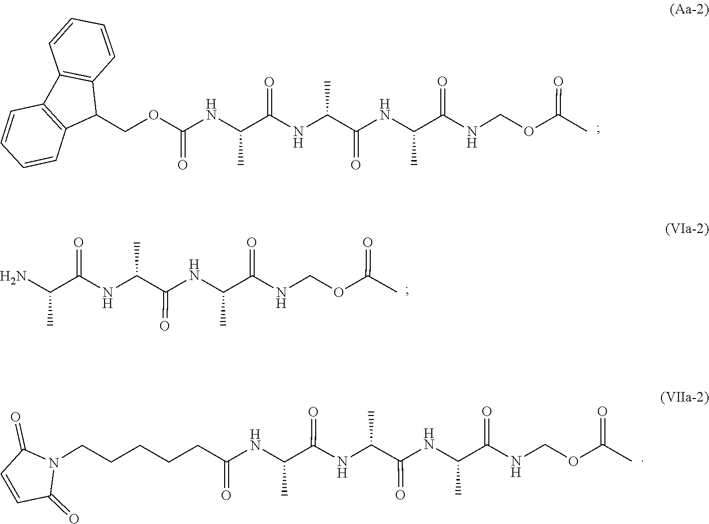 [SMILES: CC(=O)OCNC(=O)[C@H](C)NC(=O)[C@@H](C)NC(=O)[C@H](C)NC(=O)OCC1c2ccccc2-c2ccccc21 ; CC(=O)OCNC(=O)[C@H](C)NC(=O)[C@@H](C)NC(=O)[C@H](C)N ; CC(=O)OCNC(=O)[C@H](C)NC(=O)[C@@H](C)NC(=O)[C@H](C)NC(=O)CCCCCN1C(=O)C=CC1=O]

47. The method of any one of claims 32 to 41, wherein the compound of Formula (A) is represented by Formula (Ab) or (Ac); the compound of Formula (VI) is represented by Formula (VIb) or (VIc); and the compound of Formula (VII) is represented by Formula (VIIb) or (VIIc):

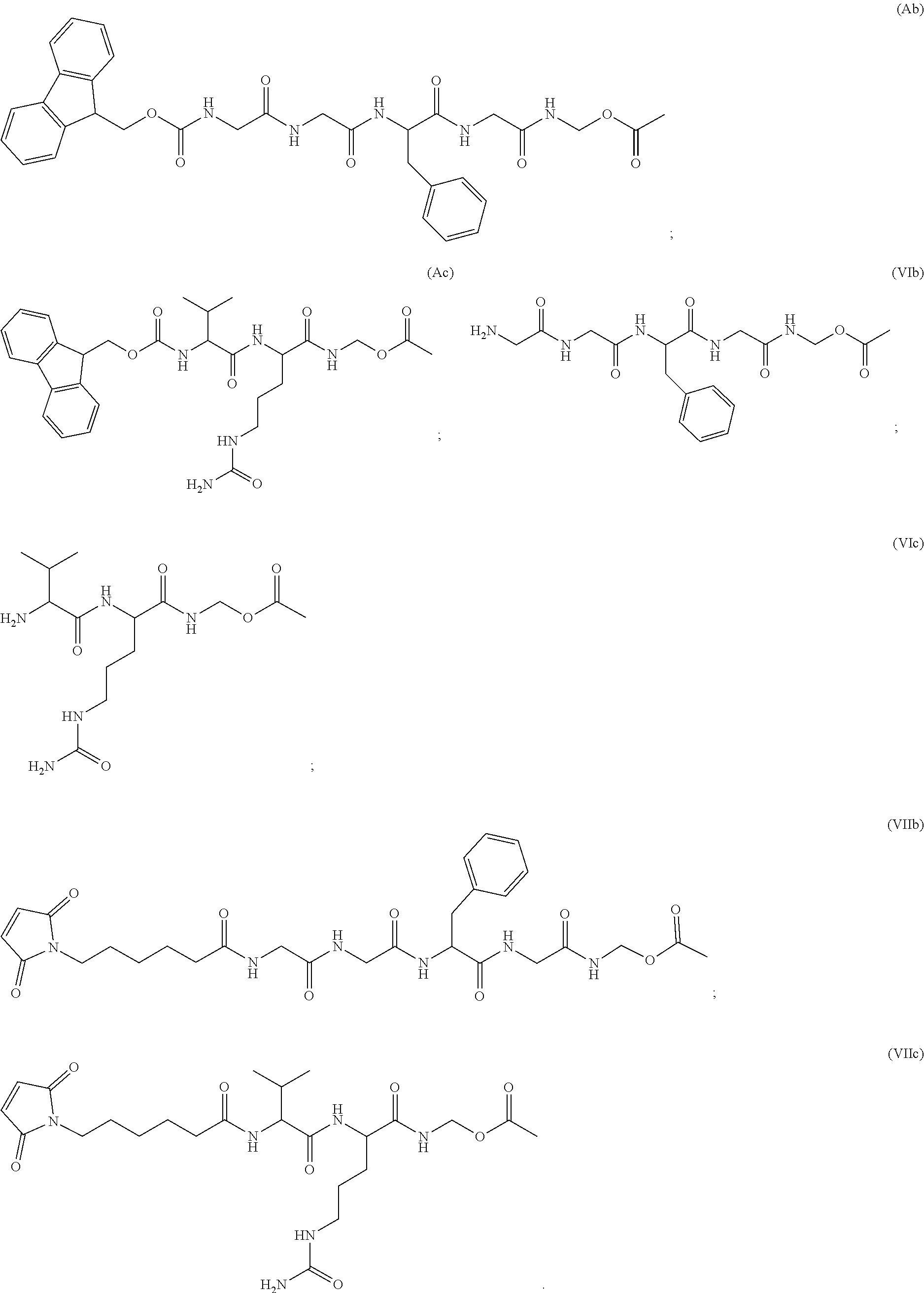

48. The method of claim 47, wherein the compound of Formula (Ab) is represented by Formula (Ab-1); the compound of Formula (Ac) is represented by Formula (Ac-1); the compound of Formula (VIb) is represented by Formula (VIb-1); the compound of Formula (VIc) is represented by Formula (VIc-1); the compound of Formula (VIIb) is represented by Formula (VIIb-1); the compound of Formula (VIIc) is represented by Formula (VIIc-1):

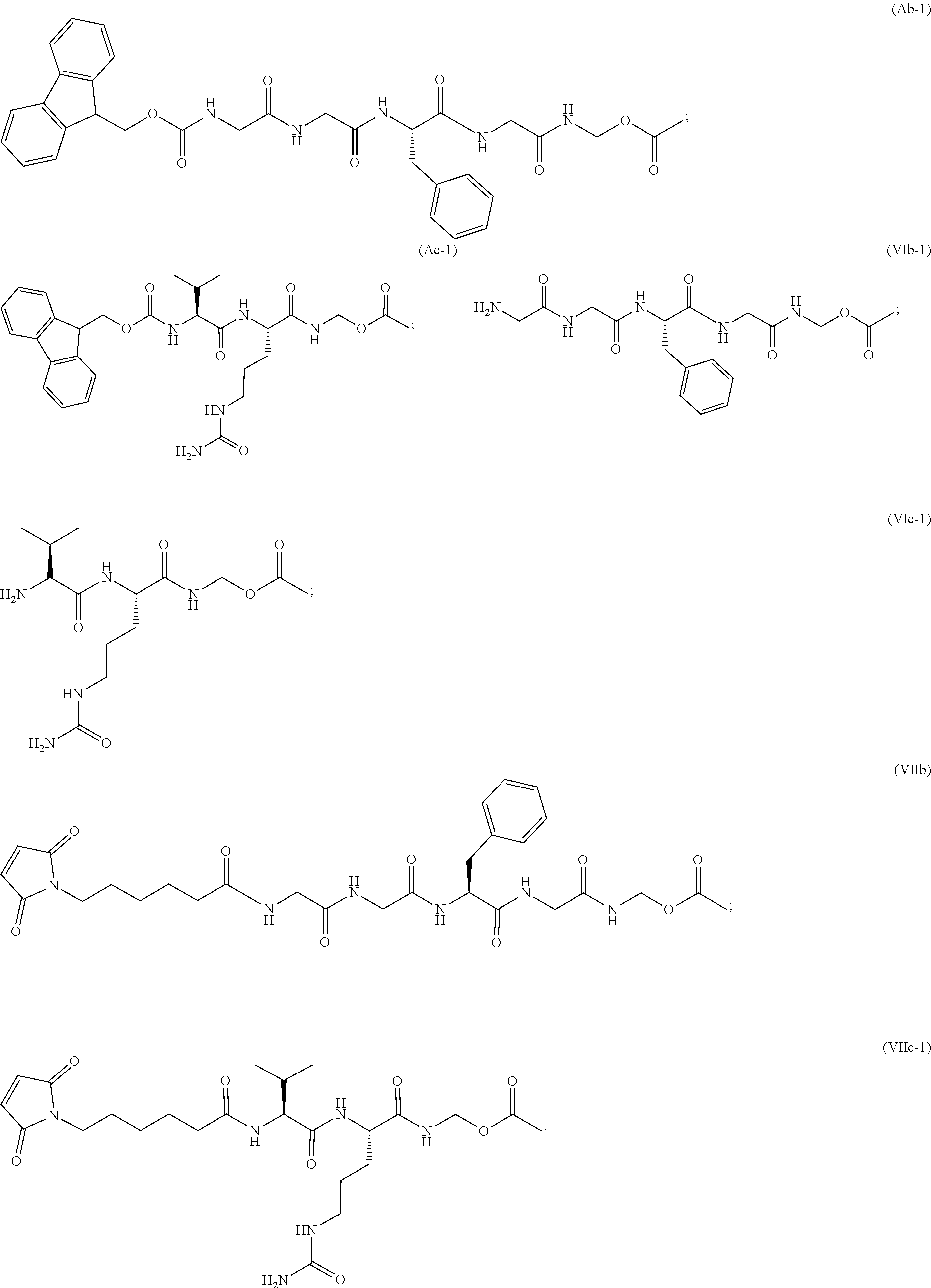

49. A method of preparing a compound of Formula (V): 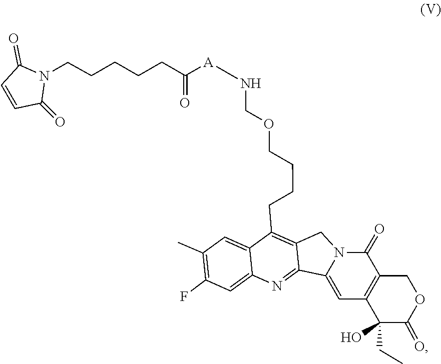
comprising reacting a compound of Formula (VII): 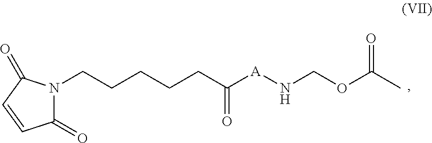
with a compound of Formula (II): 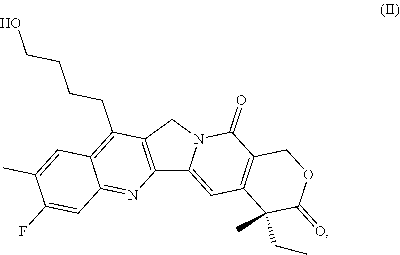 [SMILES: CC[C@@]1(C)C(=O)OCc2c1cc1n(c2=O)Cc2c-1nc1cc(F)c(C)cc1c2CCCCO]
to form the compound of Formula (V).

50. The method of claim 49, wherein the reaction is carried out in the presence of an acid.

51. The method of claim 50, wherein the acid is selected from TFA and HCl.

52. The method of claim 50, wherein the acid is boron trifluoride etherate (BF₃·OEt₂), boron trichloride (BCl₃), or aluminum trichloride (AlCl₃).

53. The method of claim 49, wherein the reaction is carried out in the presence of boron trifluoride etherate (BF₃·OEt₂).

54. The method of any one of claims 49 to 53, wherein the compound of Formula (VII) is represented by Formula (VIIa) and the compound of Formula (V) is represented by Formula (Va):

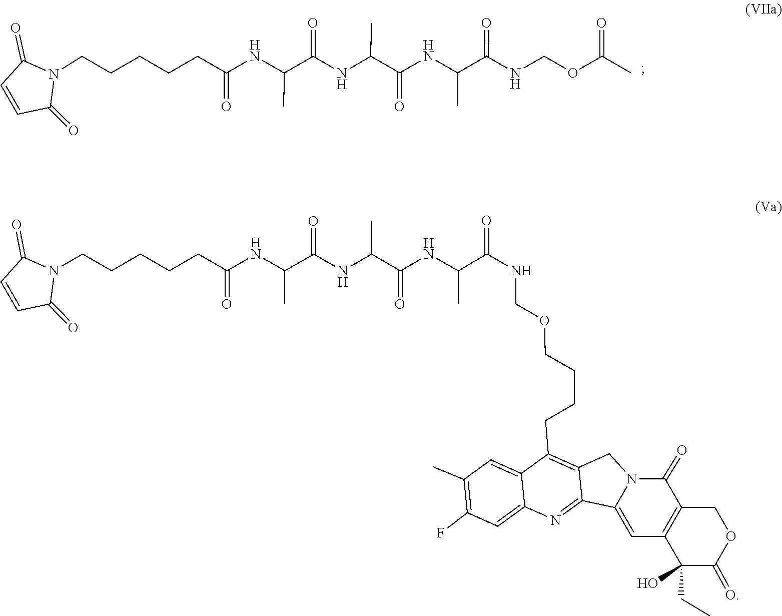 [SMILES: CC(=O)OCNC(=O)C(C)NC(=O)C(C)NC(=O)C(C)NC(=O)CCCCCN1C(=O)C=CC1=O ; CC[C@@]1(O)C(=O)OCc2c1cc1n(c2=O)Cc2c-1nc1cc(F)c(C)cc1c2CCCCOCNC(=O)C(C)NC(=O)C(C)NC(=O)C(C)NC(=O)CCCCCN1C(=O)C=CC1=O]

55. The method of claim 54, wherein the compound of Formula (VII) is represented by Formula (VIIa-1) and the compound of Formula (V) is represented by Formula (Va-1):

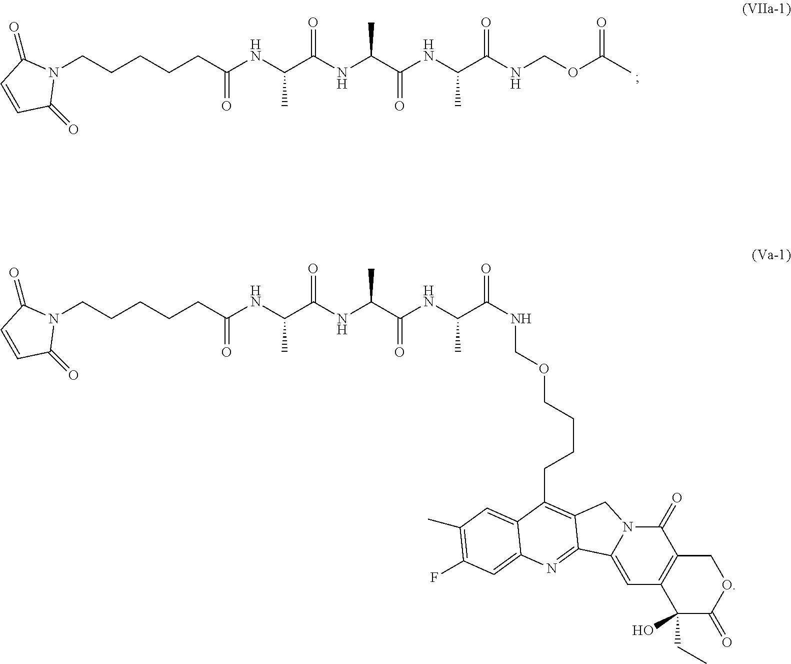 [SMILES: CC(=O)OCNC(=O)[C@H](C)NC(=O)[C@H](C)NC(=O)[C@H](C)NC(=O)CCCCCN1C(=O)C=CC1=O ; CC[C@@]1(O)C(=O)OCc2c1cc1n(c2=O)Cc2c-1nc1cc(F)c(C)cc1c2CCCCOCNC(=O)[C@H](C)NC(=O)[C@H](C)NC(=O)[C@H](C)NC(=O)CCCCCN1C(=O)C=CC1=O]

56. The method of claim 55, wherein the compound of Formula (VII) is represented by Formula (VIIa-2) and the compound of Formula (V) is represented by Formula (Va-2):

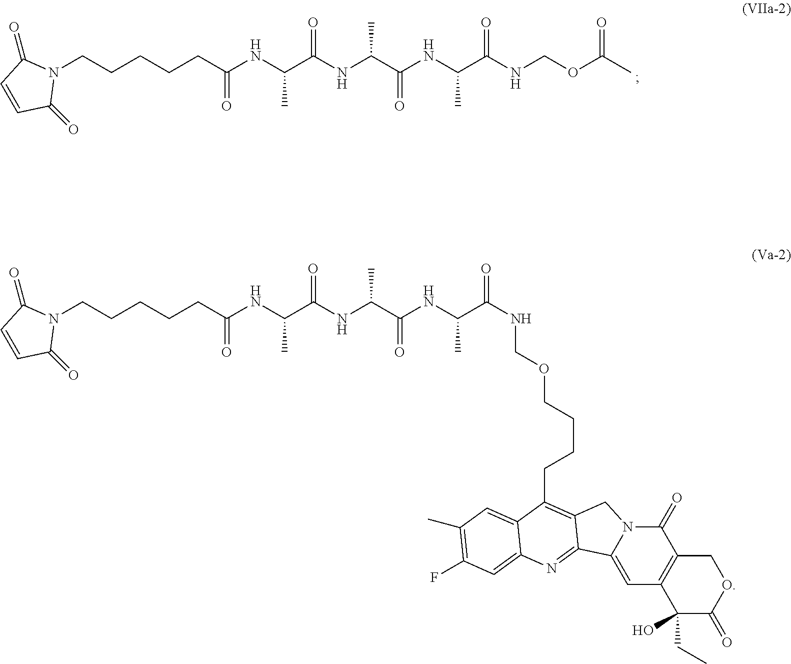 [SMILES: CC(=O)OCNC(=O)[C@H](C)NC(=O)[C@@H](C)NC(=O)[C@H](C)NC(=O)CCCCCN1C(=O)C=CC1=O ; CC[C@@]1(O)C(=O)OCc2c1cc1n(c2=O)Cc2c-1nc1cc(F)c(C)cc1c2CCCCOCNC(=O)[C@H](C)NC(=O)[C@@H](C)NC(=O)[C@H](C)NC(=O)CCCCCN1C(=O)C=CC1=O]

57. The method of any one of claims 49 to 53, wherein the compound of Formula (VII) is represented by Formula (VIIb) or (VIIc) and the compound of Formula (V) is represented by Formula (Vb) or (Vc):

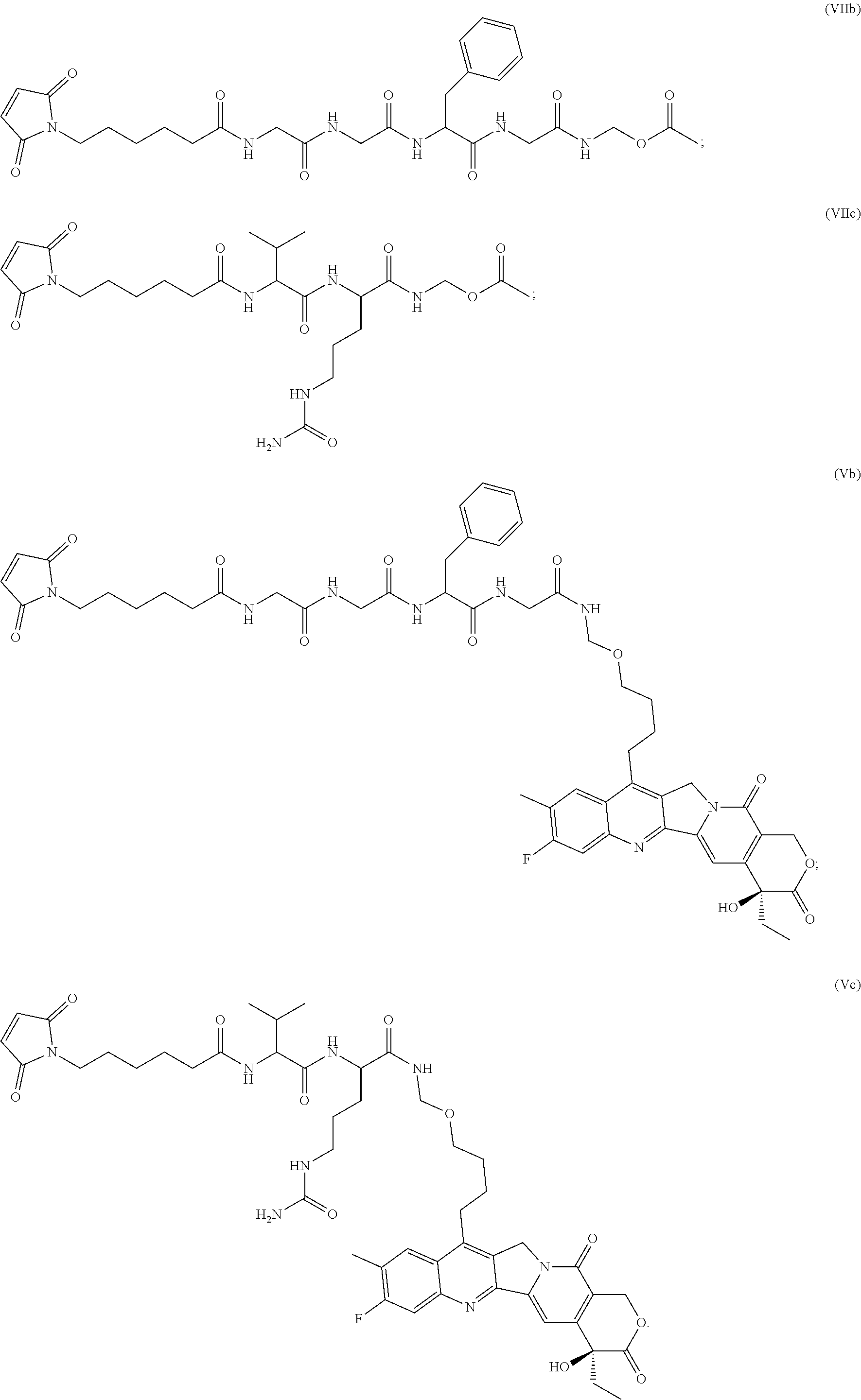 [SMILES: CC(=O)OCNC(=O)CNC(=O)C(Cc1ccccc1)NC(=O)CNC(=O)CNC(=O)CCCCCN1C(=O)C=CC1=O ; CC(=O)OCNC(=O)C(CCCNC(N)=O)NC(=O)C(NC(=O)CCCCCN1C(=O)C=CC1=O)C(C)C ; CC[C@@]1(O)C(=O)OCc2c1cc1n(c2=O)Cc2c-1nc1cc(F)c(C)cc1c2CCCCOCNC(=O)CNC(=O)C(Cc1ccccc1)NC(=O)CNC(=O)CNC(=O)CCCCCN1C(=O)C=CC1=O ; CC[C@@]1(O)C(=O)OCc2c1cc1n(c2=O)Cc2c-1nc1cc(F)c(C)cc1c2CCCCOCNC(=O)C(CCCNC(N)=O)NC(=O)C(NC(=O)CCCCCN1C(=O)C=CC1=O)C(C)C]

58. The method of claim 57, wherein the compound of Formula (VIIb) is represented by Formula (VIIb-1); the compound of Formula (VIIc) is represented by Formula (VIIc-1); the compound of Formula (Vb) is represented by Formula (Vb-1); and the compound of Formula (Vc) is represented by Formula (Vc-1):

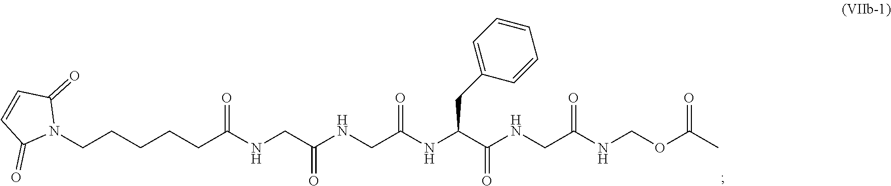 [SMILES: CC(=O)OCNC(=O)CNC(=O)[C@H](Cc1ccccc1)NC(=O)CNC(=O)CNC(=O)CCCCCN1C(=O)C=CC1=O]

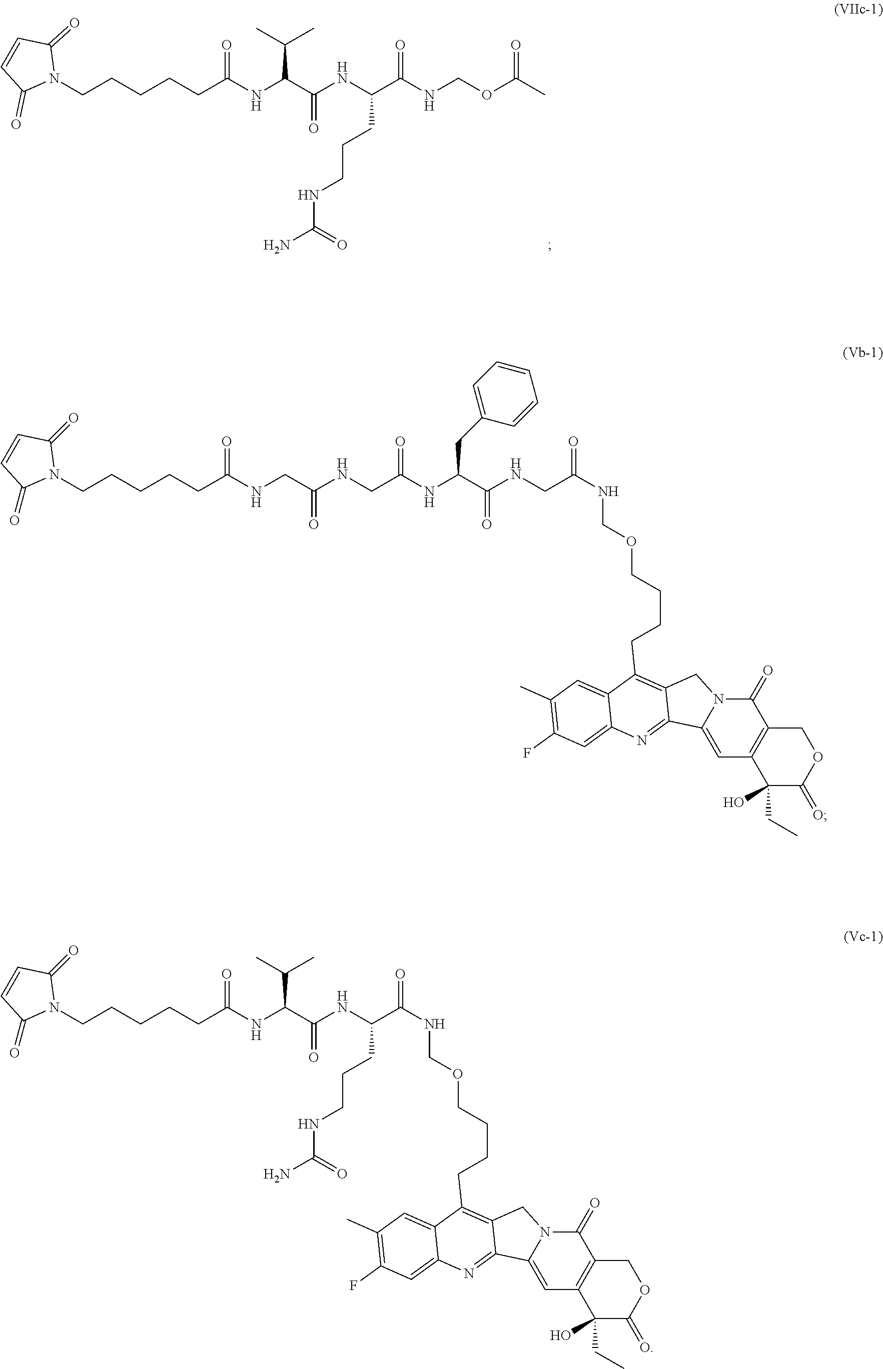

59. A method of preparing a compound of Formula (II): 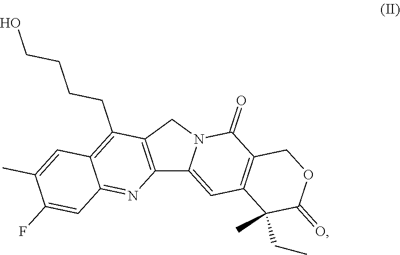 [SMILES: CC[C@@]1(C)C(=O)OCc2c1cc1n(c2=O)Cc2c-1nc1cc(F)c(C)cc1c2CCCCO]
comprising reacting a compound of Formula (VIII): 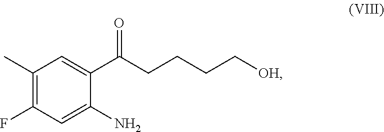 [SMILES: Cc1cc(C(=O)CCCCO)c(N)cc1F]
with a compound of Formula (C): 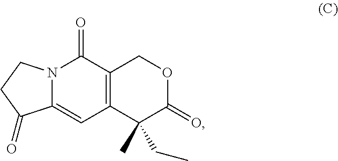 [SMILES: CC[C@@]1(C)C(=O)OCc2c1cc1n(c2=O)CCC1=O]
to form the compound of Formula (II).

60. The method of claim 59, wherein the reaction is carried out in the presence of an acid.

61. The method of claim 60, wherein the acid selected from pyridinium p-toluenesulfonate (PPTS), p-toluenesulfonic acid, methanesulfonic acid, camphorsulfonic acid, sulfuric acid, hydrochloric acid, trifluoroacetic acid, and trichloroacetic acid.

62. The method of any one of claims 59 to 61, wherein the compound of Formula (VIII) is prepared by reacting a compound of Formula (F) 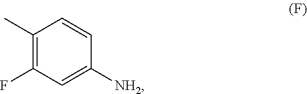
with δ-valerolactone 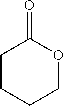
to form the compound of Formula (VIII).

63. The method of claim 62, wherein the reaction is carried out in the presence of a Lewis acid catalyst.

64. The method of claim 63, wherein the Lewis acid catalyst is selected from AlCl₃, BCl₃, BBr₃, AlBr₃, and GaCl₃.

65. The method of claim 64, wherein the Lewis acid catalyst is AlCl₃.

66. The method of any one of claims 59 to 61, wherein the compound of Formula (VIII) is prepared by a method comprising the following steps:
(a) reacting a compound of formula (G) 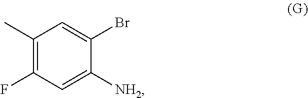
with a compound of (H) 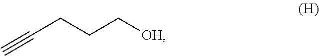
to form a compound of formula (J) 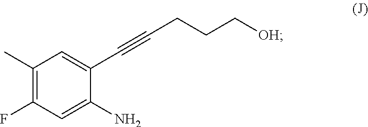
(b) reacting the compound of formula (J) with water to form the compound of formula (VIII).

67. The method of claim 66, wherein the reaction in step (a) is carried out in the presence of Pd(Ph₃)₂Cl₂ and CuI.

68. The method of claim 67, wherein the reaction in step (a) is carried out in in the presence of a base.

69. The method of claim 68, wherein the base is triethylamine (TEA).

70. The method of any one of claims 67-69, wherein the reaction in step (a) is carried out in toluene.

71. The method of any one of claims 66-70, wherein the reaction in step (b) is carried out in the presence of an acid.

72. The method of any one of claims 66-71, wherein the reaction in step (b) is carried out in the presence of H₂SO₄ and HgSO₄.
